SEQUENCE LISTING (577 sequences)

SEQ ID NO: 1 - DNA, 9726 bp, homo sapiens
gactgtggagttaagcagaacccatcaggaagtgcacaggcgtccggcgtgctcctccctccctgcagccccgggcagcatctcccagaggctccgcggcccaggctcctggtgtgtctgcagtgcaggtggctcctggaagaccctcaggtgggtgggtctggtgctttctgaaaggtggccaggaaggttggcgttggcctagttcaggatcctggcactgatcccccagagcagcctgaggcttcccacaggcagccaggcagcctccgccatgcggccccgtccctttcacggcccctgggcggagatgtgttcatttcagtggaattacggtggcagatgggaccctgcatttcaagctggggaagcaggaaaggggcccagtgttcccaggactgtctaccgtgtggccagtgtttttattcagtcccttgtctgtcaaaactgtcctgaatggggtgacaaagtgcagcgggaagttgagaaatgcctcaaaagaccaagcctggggcaactatgtctccaggagcagtgacccagctcctctgggctggaaaccagcgtttcccagcggggctggaggctgtctgaggaccacgtggctgctcccagccttgccgcagtcccagtctcagagggagtggctttcagcggctgaggcccccaccatgctggcagaaagggacagcagtggctggtcccaacatgggtagttctgttgcagccactgggatgggggccgggcctcatctggggcaggagaagagtccagggattcactggtgcccggcacctcaacctggagaaaacaccgcagcagcactcctggccagccaagggcaacactgacagcccctcccagggttgcggggccggcagggggtgacagagacacgctcctgtgggcagctcttcccggccagtgcagcgaccccgcagctgcagcagggctgggtggccactcttgcatcatcagcagccggccgtggaggagggagggcacagccagggatgtgagcaggcgtcggtcaggggaatgaggaagtcccacaagaaccggctcccccagctgctgtgctggtgggagggccactggccaacctgctctcacccagcttgggctggccatagtgccaggcagttgccctcagctcctctgcccattccagccatggaggtggctaaccttgcaggctccagggcgtgatgggagtgtccaggaggcagaggccagagggggtcatgctgtagggggagttcctctctcagaccccatctgtgcctcccccgctgcgtatctgggtgacatggatgccaacgggcaggagcaagggctgggaaaggctgcggaggggccctcggctggtctggtggggagcggggtcactgtgtctctgctgcctccctgggggatccagcgtggggatgggacatggagctgggcacctctcctggatgcccctgggctgatccccctcgatgccctgacgagagccctggcccagcgtctgagaaggagtgggagctgggcccgggtgggggcaggagccggcatcgctctcagcactgccccacactccccagcctgcctgctgaggccatgtcggactacgagaacgatgacgagtgctggagcgtcctggagggcttccgggtgacgctcacctcggtcatcgacccctcacgcatcacaccttacctgcggcagtgcaaggtcctgaaccccgatgatgaggagcaggtgctcagcgaccccaacctggtcatccgcaaacggaaagtgggtcagtgttgccccgcgggccccaggccccaaacccaaccacagagtcagctcggaggtggctgcacgctctgccaccctgaccctcaaccccaggagagccccgcctggctttgcccctggcctctgactgaggcccaacctcaaccctggcacgtggcccgtcccccagcctctcctgccggcttggtgctgccaagaggggcagcggggctcccaccgcagcgggggctcctgagacagccaaggggcgggtgggcagcacttggtccccagcctagtaccaagacccgcagggtacctcatccaagacccagaccacggcacacctcatctgcatgccagcctccacctgtccctgcccccaggcccacccaccccagccctctgcctcgcctgtgccccgcccccagttaggcctctctggcccgcaggtgtgctcctggacatcctgcagcggaccggccacaagggctacgtggccttcctcgagagcctggagctctactacccgcagctgtacaagaaggtcacaggcaaggagccggcccgcgtcttctccatgatcatcggtgagtgacgggggaggcggggccgacaggggaggcgctggcggggagcgggcagtggtgttgggtcctctgcctggcccccatccctaggagccctgagctcctgccacagcgcagggcgtgatggagctgggccaccggggaacacggcaccatctgacactggcctggcccacagacgcgtccggggagtcaggcctgactcagctgctgatgactgaggtcatgaagctgcagaagaaggtgcaggacctgaccgcgctgctgagctccaaagatgacttcatcaaggagctgcgggtgaaggacagcctgctgcgcaagcaccaggagcgtgtgcagaggctcaaggaggagtgcgaggccggcagccgcgagctcaagcgctgcaaggaggagaactacgacctggccatgcgcctggcgcaccagagtgaggagaagggcgccgcgctcatgcggaaccgtgacctgcagctggaggtgcgcccgctgcccccgggaaaccaggccaccctcgcctggcctggcggggtctgcaggccggggcggtcgggggtggccccacgggtctgcggtcaccgcctggaggggacagccaggctcactggaccttctgcagattgaccagctcaagcacagcctcatgaaggccgaggacgactgcaaggtggagcgcaagcacacgctgaagctcaggcacgccatggagcagcggcccagccaggagctgctgtgggagctgcagcaggagaaggccctgctccaggcccgggtgcaggagctggaggcctccgtccaggtggggccgtaggcagggcaggtgcagcccaggcagggcagtgggggccttcccagccaggccattgccttttgcccccttcaggaggggaagctggacaggagcagcccctacatccaggtactggaggaggactggcggcaggcgctgcgggaccaccaggagcaggccaacaccatcttctccctgcgcaaggacctccgccagggcgaggcccgacgcctccgggtaggagggcaggcggggagacatggcccctggggtccaagccaggctggacgggctgagctccgcccactccgtgcctggaaggtggggtgtggagaccccagtgcagcgcctccactcacccactcctggaggcacagtggacggtgccctctccttgcccgcctgccccccatggcaaaggccaacctctgggctcatggtgcctgaggcacagcagtgtctgaggcctgccggagggccctgtcctgccgcggtctggaagtgcagggctggtaggggcctggggactcctatggcctcccccgacatcccaactggcctgaccacatgcttctgagcccctgcccggagtataccccaagggccagaatggggggcctcaggcctgtgggggctgagctgccgccccctgcagtgcatggaggagaaggagatgttcgagctgcagtgcctggcactacgtaaggactccaagatgtacaaggaccgcatcgaggccatcctgctgcagatggaggaggtcgccattgagcgggaccaggtgggcactcgcaggggcaggacaaagcaccccactcgcgggtgggtgcaccagggccacggcccttgagggcagttgtcagaggatttttaatctttgttatttgtttttgagatggggtcttgctatgtcacccagggtggcctcaaactactggcctcaagcaatcctccggcctcagcctcccaaagtgctgggattgcaagctgagctgccacacctggtctgttttttaaatgacagaagcaatacttaacactaaaaaaaagtaaacgatgatgaagtcaatacttccccaccccagcctactgtcagggaaaaccgtgttcaaccttcccgctccaccttacagacttacctgtcatctatccatgtatccatctgtctatcatctatcacctatctatctatttatatatttggttttgttttacaaaaatggaaacattctgtcttgttttaccagtagctttttttaatcttaaaaaaagcttggctggacgtggtgtctctcgcctgtcatcccagcactttgggaggctgaggcgggcggatcacttgagcccaggagttccagagcagcctgggcaacacggtgagatccctgctctacaaaaaaatgcaaaaattggcctggcgcggtggctcacgcctataatcccagcactctaggagggcgaggcaggcggctcacttgaggtcaggagttcgagaccagcctggccagtatggggaaaccccgtctctactaaaaacacaaaaattagcctggtgtggttccgggcgcctgtaatcccagctactcaggaggctgaggcaggagaattgcttgaacccaggaggcagaggttgcagtgagctgagatcacgctactgtactccagcctgggtgacagagcgagactccgtctcaaaaaaaaaaaaaattaactggtcgtggtggcgcatgcctgtggtcccagctatttaggaggctgaggctgcagcaagtcaccattgcgctattgcactccagcctgggccacgggagtgaggcctgcaaggcctccagtgctacgtgctgtgagccggatggagcgtcgcggggcagggctgtgtctgcttcccatgccccgtcccaagtcccatcctggaggttgtttccaatggacaccattgctgcttcggctaaatgctggatggcactttgtccacatcaggagccttaaaacgagacccctggggctacaaaggaagccccggcccccatgagagcagcccaggctcagagctcctgtctcagccccacctcagcctcactcgcgcctggcccacgccaaggcaggggaaaagggcagatccagctgtgcccccatgggggttggtgtggctcccagggcgggggctcccacgggacttcccgggagcgcgttggccagggattagggaagctgccagccgttgctccagtctgggcagaaggggttaggtgagggcctcccagcctcggctgcaggggtggtcaagccgtcgtgtttgcacattcgctcctgcgtctcagtgtccgcagtgaggacaggggtggtaaagagtgctgcggtcacgaaggacgcttagctatggacagtcagggtctccatatcaagccgcgtccaaaatttatcaaatgtgcacgtgtgggggtacatagcacacgctgtgtccaccgggggtgcaggcttgggaggagacagaagggcagcagggacaactgtcacatttacaatcccccacgtgctgacttcgtgtccgcagagcacacaaatggaggggctgtgaccagcctccgcgcccagcggcttgacgtcctccggagcctctgcttggagttgggcggccgggccgagggcccagggcaagcttggggccctcactgagggtcggccttgtgctgtcccgtcaggccatagccacgcgggaggagctgcacgcacagcacgcccggggcctgcaggagaaggacgcgctgcgcaagcaggtgcgggagctgggcgagaaggcggatgagctgcagctgcaggtgttccagtgtgaggcgcagctactggccgtggagggcaggctcaggcggcagcagctggagacgctcgtcctggtgggcctggggcctgcggcagggaggggagccggcgcgtgggatgcagggccaggggagcacggagcctcagccccggccttcccgacccaacccttctctgtgacgggccccaacctaaccaaacccctcctggcagtggggtgagggcacacgagcagttcagggtcccagcaggaagtggggctgcagggccggggtgggtcctgggcctggccatcaggcagcctagcaggttgttctgggcatggagggggcctggtgtggctgagggcatgcccagggctccctggaggatcccgctctgtgccctgcccaccctgtgcctggggagccctctgccctcacagcccacccaccccattttctatgaccacagagctccgacctggaagatggctcacccaggaggtcccaggaggtgagtgagaccccagccagcacgaggctggccttccttcacctggggtcctggctgagctgccaccaggcatagccccacactgcccgtcacatggccctgctcggtgggctgggctgggtggaggaagagcaggtggccaaatgctaaggcctctgatggctcaaggggtaaggtaaggcacggacagtgcccacctcctggccgcccaatgtccgtctgctgggcacctgagtctctgagaagctctgtgcagggggtgacggcaatggctgcacccagctgagggccaagggctggggtgggcagtgtggccgggtccagtccttggcgaagtggcctaatgggacatcttgactctctttggaagctctcactcccccaggacctggaggacacccagctctcagacaaaggtgaggtggggacagcagtacacccagaggcctcccagcttccccaagtgaggcccatcttcagcacaggcagcccgtgccgcaatgcctgtgtcaacccccacaacccccactccacagacagggaaactggggctggggataagtaaaatggtcaaggtcacctagagaacaaactacagagcccagctggaacccagggccccagctagtgggacaagtgtcccctggggtggagagtgtgggggtctgggcagcccccatgggctgcagaggtgagcaggcccaccgtcctgagagtgtggggcccctctgggcagcccccacggggtgcagaggtgagcaggcccagtcctggctgctccaagcttcctcctggacagagggaggtggtaacgagcgaacgaccaggtgcatgaagctagaagatggggcgagatggggccccacgtggcttgagggaaagggctgaatgcaggggtggccaagggcctgctgtgtcccggtgcagtgggggtaaggcagtggcaggggcttctagcgggcagggcaggccccctctgcctgtctgggggaaggactgggtggctagctgggagcagctcccgagtgtccaggagtgggcagtggtgatgaggtaggtgtttgcactgaaggtgctgtcaggtaggggttggggagggcccagcagaggggtcctgggaggctgcaccggagttggacaagcttcctgagtaccaggccgtggcctccaagcagaggcggaaggggaaggaggagccgcagacggggaagtgaagaccagcgcctttggctgcagctcagaacacttgaacatggggatgccaagcccccatgtaggcacagaggcgagcaggacccaggagtccggctcaggcactgcccccatccccctgggtggctgtggcaggagcaggggcaggtgtgaaggttggagggtgagcaggggaggggctggctctaaggggctgccaatggccaggacagccctggcctggccctcaccgcagtggggctgagactggacgtgcgtggtttctgcccaagggtcctgatccctggtctgcccaggtcctggcggtttctgagggggtccagccgcctgaaggttctctcccaattgctgcagtacccctgcccccacttcaggaggacccagacagccactgtagctgtccccaactctctccccagggccgccatgcccacagatgctttggagtgatgggggacggtgctgctcaggcccagggtttaagttcccccgaggtctgggaggcgttcctccctggcaggcagccagctcgggggagggatggtcactggcgggagcgttgggcctgggctggcatctggcggcctcctggagcaatgaacaggcaagcgaggccataaggcctcagtctcatctacagaaagcagagtctacactggacccatgtggggatcccaccaggccttggaagacagccccactgcaatgccggggaggggtggggcagccgtgctatacgtggcccagcagatgggcaggtcccacaggcacgactctcctttccaggctgccttgccggcggggggagcccgaaacagccctttgcagctctgcaccaggagcaggttttgcggaacccccatgtaaggcttccccggggtggggtcctcccagccgtgggcctcagggtgaccgatcacagggagagtggctccctgccctgggcaccccctgcggtggccccgacgacagctgaggagtgaccacaaggtctctgcccacagtgctcggggtgcggtgtctgggctgcgaagtggatccccctttcttgggcactgcagcagcttggggggctttttggacgtggatgtgcctggtcctggttttccgagggcctttacagtggatgaggaggtgaacacaggagtcctgagagcaagcaccacctcgggctttgttgtagaaacaatggcccggaccccaggctggagccgtggcttggcctcctgggtgtgtcttggcatctgaaatgcaggctacccacaccggctcacctccaggggtacaggcaggtcccacagggagagcttggcgctgagctgaggctgtctgggctcctcgcctcccaaccagtctgcagttacaggggccagtggggggcgggtgagaaggacgggttccctcaggggagccggccggagcccgagccttcccccttctccaggacgcaggcctgagcagcggggagccgcccgagaaggagcggcggcgcctcaaagagagttttgagaactaccgcaggtaggcgggcggcccccaggcttctccaaaacgggctggggagcggcagccgggtcgcgcctgagacacctgtcttcacaggaagcgcgccctcaggaagatgcagaaaggatggcggcagggggaggaggaccgggagaacaccacgggcagcgacaacaccgacactgagggctcctagccgcagcagcgcaggccccgaccagggcacacccaccggcccggcctcctgccacccgggggtgccgacgccctggggcgcagacttccccgagccgtcgctgacttggcctggaacgaggaatctggtgccctgaaaggcccagccggactgccgggcattggggccgtttgttaagcggcactcattttgcggaggccatgcgggtgctcaccacccccatgcacacgccatctgtgtaacttcaggatctgttctgtttcaccatgtaacacacaatacatgcatgcattgtattagtgttagaaaacacagctgcgtaaataaacagcacgggtgacccgca

SEQ ID NO: 2 - DNA, 2123 bp, homo sapiens
gactgtggagttaagcagaacccatcaggaagtgcacaggcgtccggcgtgctcctccctccctgcagccccgggcagcatctcccagaggctccgcggcccaggctcctggtgtgtctgcagtgcaggtggctcctggaagaccctcagcctgcctgctgaggccatgtcggactacgagaacgatgacgagtgctggagcgtcctggagggcttccgggtgacgctcacctcggtcatcgacccctcacgcatcacaccttacctgcggcagtgcaaggtcctgaaccccgatgatgaggagcaggtgctcagcgaccccaacctggtcatccgcaaacggaaagtgggtgtgctcctggacatcctgcagcggaccggccacaagggctacgtggccttcctcgagagcctggagctctactacccgcagctgtacaagaaggtcacaggcaaggagccggcccgcgtcttctccatgatcatcgacgcgtccggggagtcaggcctgactcagctgctgatgactgaggtcatgaagctgcagaagaaggtgcaggacctgaccgcgctgctgagctccaaagatgacttcatcaaggagctgcgggtgaaggacagcctgctgcgcaagcaccaggagcgtgtgcagaggctcaaggaggagtgcgaggccggcagccgcgagctcaagcgctgcaaggaggagaactacgacctggccatgcgcctggcgcaccagagtgaggagaagggcgccgcgctcatgcggaaccgtgacctgcagctggagattgaccagctcaagcacagcctcatgaaggccgaggacgactgcaaggtggagcgcaagcacacgctgaagctcaggcacgccatggagcagcggcccagccaggagctgctgtgggagctgcagcaggagaaggccctgctccaggcccgggtgcaggagctggaggcctccgtccaggaggggaagctggacaggagcagcccctacatccaggtactggaggaggactggcggcaggcgctgcgggaccaccaggagcaggccaacaccatcttctccctgcgcaaggacctccgccagggcgaggcccgacgcctccggtgcatggaggagaaggagatgttcgagctgcagtgcctggcactacgtaaggactccaagatgtacaaggaccgcatcgaggccatcctgctgcagatggaggaggtcgccattgagcgggaccaggccatagccacgcgggaggagctgcacgcacagcacgcccggggcctgcaggagaaggacgcgctgcgcaagcaggtgcgggagctgggcgagaaggcggatgagctgcagctgcaggtgttccagtgtgaggcgcagctactggccgtggagggcaggctcaggcggcagcagctggagacgctcgtcctgagctccgacctggaagatggctcacccaggaggtcccaggagctctcactcccccaggacctggaggacacccagctctcagacaaaggctgccttgccggcggggggagcccgaaacagccctttgcagctctgcaccaggagcaggttttgcggaacccccatgacgcaggcctgagcagcggggagccgcccgagaaggagcggcggcgcctcaaagagagttttgagaactaccgcaggaagcgcgccctcaggaagatgcagaaaggatggcggcagggggaggaggaccgggagaacaccacgggcagcgacaacaccgacactgagggctcctagccgcagcagcgcaggccccgaccagggcacacccaccggcccggcctcctgccacccgggggtgccgacgccctggggcgcagacttccccgagccgtcgctgacttggcctggaacgaggaatctggtgccctgaaaggcccagccggactgccgggcattggggccgtttgttaagcggcactcattttgcggaggccatgcgggtgctcaccacccccatgcacacgccatctgtgtaacttcaggatctgttctgtttcaccatgtaacacacaatacatgcatgcattgtattagtgttagaaaacacagctgcgtaaataaacagcacgggtgacccgca

SEQ ID NO: 3 - DNA, 11989 bp, macaca fascicularis
  misc_feature (6569)..(6578): n is a, c, g, or t
ggcagggtgctgggggcccagcactggggtcctcgaggcctcctgtaccctggctgccctcccagcagagaagcaggctttcgcccatttcatggaaagcacctgtggctttgttcttcatgctgagccggcaccctccccagatggctgccagccctcgcaccgccccagctgacccagggggttccctggaacaggtgacctggctacccagacacgatggcagcccccacaactttcactgtccttttgcgtgggcctttcttggcataagaccagcccccatcttctccgtctgtcctctcactccctttccttcgtctctgtggcagcagcgcccccaggccctttgctctctgggtttcactccctcactggaagcaccgtggggcctcagccctcggcacccggcggacaggacggtcccatggccggccgcgctcctgagcatgtgttgctgtgcctggttctctagcaactggtgcctagtctcctggtggccactctgacctgtgggttatttagtggtatattatttaatttccaaatatttagggattttccagatacctgttactgatttttagcttccgacacggttagagaacatggtatgtgtgttaattcctaaggacggacaccacagggaaatcactggggtccctcgttttacaagctggggaatcactgggtcatgagggcctggcgccagctcgggcccactccccagcagggcccaggacggcgttcctgcctgcacactcgctcatgctgctggtgaggggcccagcctggagtgtggcattcccgagagccccagaggacccctaactccactcctcactgcgtgatgtcaggcctgagggccgggggctgggaacggttaggcgagctccgcaccagcttcctggccttccagcctggcagcccccttctcgaagctgttgggtggcaactcggttcgacccgctttaccggtgaggcggggcctgggggctccccagcctgcagggatgaggctccagccgcccccacgccgaagtgcacagttcagggcagactctgctgcccgtcgtctgtggatggggggcgggctgtgccctgccatcaccggtgcttctgaagctggtttgggggaccctgggagtggcctcagtcctcacgtgcatcctggagaagagcaaggatgcctcgtgacaaggataatgatatcgagcgacacttctgaaagggacgcttctcccccgtgtgcagctgggtggtggccggtgccatggcccctggcaggctgcagtggcctcctgggctccataaggtgggctgcagccctttcatgaccaaggcgtgtgctgggcatgctgtgtggctgtgtgcccccctgcacatgccctgccccgctgtcctgagatgccggcgagggcaagggtgtccgcacagggcagagcatgccctcctgaagaagcctccagaaacctatgaccaggtcaaagcggctgggtgcggtctggcctcgtccagcctcctgggggtctctgctgtctcttctacttgccaggtgcccgggctgggcctctggggcattctcaggacccctggcccccggccaggctgggagggctgagagcagttttcctgtggagggcagaggacttccacttccgtctgcctctcagggcctcctgggctgcacgaatcagaaggcaggccaccagggactgtccccctgtgggaggactggcgggactgtggagttaagccatcaggaagtgcacaggcgtccggcacgctcctccctccctgcagccccaggcagcatctccccgaggctccgcgtcccaggctcctggtgcgtctgccgtgcaggtggctccttggaagaccctcaggtgggtgggtctggtacttttctgaaaagtgaccaagaaggtcggcgccggcttcgttcagaatcctggcgctgatccccgcagagcagcatgaggcttcccacaggcagccaggcagcctccgccgcgctgccccatccctttcatggccactgggcggagacatgttcatttcagtggcattacggtggcaggtgggaccctgcatttcaggctggggaggcaggaaaggggcccagtgttcccaggactgtccgctgtgtggccggtgtttctattcagccccttgtctgtcaaaactgtcctgaatggggtgacaaagtacagtgggaagttgagaaattcttcaaaagaccaagaccaaacacttcaaaagaccaagtgtctccaggagtagtgacccagctcctttaggcaggaaaccagcatttcccagtggggctggaggctgaggaccggggacctgcgtgggaggcaggaccatgtggctgttcccagccttgccccagtcccagagggagtggctttcagcggctgaggcttccaccgcgctggcagaaagggacagcagtggctggtcccaacatgggtagttctcttgtttcagccagtgggatgggggtcgggcctcatcgggggcaggagaagagtccagagattgacgggtgcccggcacctaagaacctggagaaaacagcacagcagcactcctggccagccaagggcaacaccgacagcccctcccagcttgcggggccggcggggagtgacagaggcacgctcctgtgggcagctcttcccggccagtgcagcgaccctggcagctggggcagggctgggtggccactcttgcatcatcaacagccggccatggaggagggagggcacagcgcgggatgtgagcaggcgtcggtcaggggaatggggaagttctgccagaaccggctcccccagctgctgccctagtgggagggtcattggccaacctgctctcacccagcttgggctggccgtagtgccaggcaggtgcccacagctcctccacccatggaggtggctgcccctgtaggctccagggcgtggtgggagtgtccaggaggcggaggccagaggcgagtcatgccgtggaggcagttcctctctcagaccccatctgtgtctcccccgctgcatatctgggtgacacggatggccaaggggcaggagcaggggctgggaaaggctgtggaggggccctcggccggtctggcggggagcgggtcactgtgtctctgccgcctccctgggggatccagcatggggatgggacatggagctgggtacctctcctggatgcccctgggctgaccccctcgacagccctgacgagagccctggcccagcgtctgagaaggagcgggagccgggcctgggtgggggcaggagccggcgtcactctcagcggctgccccacaccccccagcctgcctgcgaggccatgtcggactacgagaacgacgatgagtgctggagcgtcctggagggcttccgggtgatgctcacgtcggtcatcgacccctcgcgcatcacgccctacctgcggcagtgcaaggtcctgaaccccgacgatgaggagcaggtgctcagtgaccccaacttggtcatccgcaaacggaaagtgggtcagtgctgccccgcaggccccaggccccaaacccaaccaaggagcccgcttggaggtggctgcacgctctgccaccctgaccctcgaccccaggagagccccgcctggctttgcccctggcgtcccagtgaggcccaacctcaaccctggcacatggccaccctatccccctgcctctcctgccagctcggtgctgccaagaggggcagtgggactcccaccacagcgggggctcctgagacagccaagggatgggtggacagcactttgtccccagcctagtaccaagacccacagggtgcctcatccacgacccaaaccacagcacacctcatctgcatcccagcctccacctgtccctgcccccgggcccacccaccccagccctctgcctcgcctgtgccccgcccccagttaggcccctctggcccgcaggcgtgctcttggacatcctgcagcggaccggccacaagggctacgtggccttcctcgagagcctggaactctattacccacagctgtacaagaaggtcacgggcaaggagccggcccgtgtcttctccatgatcatcggtgagtgacggggaaggcggtggtgggggcgggcagtggtgctgggtcctctgcccagcccccatccctaggagccctgagctcctgccacagcgcagggcgtgatagagctgggccaccggggaacacaggcaccgtctaacgctggcgcggcccacagacgcgtccggggagtcaggcctgactcagctgctgatgaccgaggtcatgaagctgcagaagaaggtgcaggacctgaccgcgctgctgagctccaaggatgacttcatcaaggagctgcgggtgaaggacagcctgctgcgcaagcaccaggagcgggtgcagaggctcaaggaggagtgcgaggccggcagccgtgagctcaagcgctgcaaggaggagaactacgacctggccatgcgcctggcacgccagagcgaggagaagggtgccgcgctcatgcggaaccgtgacctgcagctggaggtgcgcccactgcccccggggaaccaggccccccttgcctggcctggtggggtctgcaggctggggcggtcgggggtggccccgtgggtccgttgtcaccgcctggaggggacagccgggctcaccggaccttccgcagattgaccggctcaagcacagcctcatgaaggctgaggacgactgcaaggtggagcgcaagcacacgctgaagctgaggcacgccatggagcagaggcccagccaggagctgctgtgggagctgcagcagcagaaggccctgctccaggcccgggtgcaggagctggaggcctccgtccaggtggggccgcaggcagggcgggtgcagcccgggcaggacagccggggccttcccagccaggccattgccttttgcccccttcaggaggggaagctggacaggagcagcccctacatccaggtactggaagaggactggcggcaggcactgcgggaccaccaggagcaggccaacaccatcttctccctgcgcaaggacctccgccagggcgaggcccgacgcctccgggtaggagggcaggcagggcgacgcagctcctgggggccaagccacgctgggtgggctgagctccgcccactccgtgcctggaaggtggggcctggggactcctatggcctccccccacatcccaactggcctgatcacatgctcctgagccccttccccgagtacaccccaagggccagaatggggggcctcaagcctgtgggggctgagctgccgccccttgcagtgcatggaggagaaggagatgttcgagctgcagtgcctggccctgcgtaaagactccaagatgtacaaggaccgcattgaggccatcctgctgcagatggaggaggttgccattgaacgggaccaggtgggcacctcacggggcgggcaaagcaccccactcacgggtgggtgcacagggccacaccccttgagggcagttgtcacggagtttttaatctttgttatttgtttttgagatggggtcttgctatgtcacccagggtggcctcaaactactggcctcaagcagtcctccggccccagcctcccagagcgctggagttgcaggctgagctgccacacctggcctgctttttaaatgacagaagtaatatttaacattaaaaaaaagtaaacagtgatgaagtcaatacttccctaccccagcctgctctcagggaaaaccgtgttcaaccttccggctccaccttacagacttttacctgccatccatctatccatccatccatccatctatcatctatctatttatctatcaatcatctatcatctatctatcaatctatcatccatctatcaatcatctatcaatcatctatcatccatccatctatctatcatctatctatctattatctatcatctagctatctatctgtctatcatctatctatctattatctatctatcatctatctatcatctctctatctattatctatctatctatcatctatctatcatctatctatctattatctatctatctatctatctatcatctatctatctatctatcatctatctatcatctatctgtctatctgtctatcatctatctatcatctatctattatctatctgtctatcatctatctatctatcatctatctatcgatctatcatctatctatcatctgtcatctatctgtctatctatcatctatctatcatctatctatcatctatcatccatctatctatcatctatctatctattatctatcacctatctatctatatatctttttgattttatgaaaacattctgtcttgttttaccagtagctttttttttttttttttttgagacggagtctcgctctgtcacccagactggagtgcagtggccggatctcagctcactgcaagctctgcctcccgggttcccgccattctcctgcctcagcctcccgagtagctgggnnnnnnnnnncacccgtctcggcctcccaaagtgctgggattacaggcttgagccaccgcgcccggcaccagtagctttttttaatcttgaaaaaaaacttggctggacgcagtggctcttgcctgtaatcccagcactttgggaggctgaggcgggcagatcacttgagcccaggagttccagagcagcctgggcaacatggtgagatcccaactccataaaaaatacaaaaattggccgggcatggtggctcatgcctgtaatcccagcactctagaagggcgaggcaggtggaacacttgaggtcacgagatcaagaccagcctggccagtatggtgaaaccccgtctctactaagaatataaaaattagccgggtgtagcggcgggcacctgtagtcccagctactcaggaggctgaggcaggagaatcgcttgaacccaggaggcagaggttgcagtgagctgagatcacactactgcactctagcctgggtgacagagcaagactccaactcaaaaaaaaaaaaaaaaattggcgtggtggtgcatgcctgtggtcccagctatttaggaggctgaggctacagcaagtcaccattgcgctactgcactccagcctgggccaccggagtgaggcctgcaaggcctcgagtgctacatgctgtgagccggatagagcgtcacggggcagggctgtgtctgcttcctatgccccgtcccgagtcccatcctggaggttgtctccaatggacaccactgcttcttcggcgaaattctggatggcagtttgtccacatcaggagccttaaaacgagacccctggggccataaagcaagccctggccaccatgagagcagcacaggctcagaactcctgtgcggccccaccacagcctcactcacgcctggcccaccccaagatggggaaaaaagggcagatccagtcgtgcccccatgggggttggtgtggctcccagggcgagggctcccacggggctgtgcgggggcaggtaggccagggattagggaagctgccagccgttgctccagtccagtggcaggggttaggtgagggcctcccagcctcggctgcaggggtggtcacgccgtcatgtttgcacgttcgctcctgtacctacctcagcatctgcagtggggacaggggtggtgaagagtgctgcgctcatgaaggagttttagccatggacagtcagggtccccatatcaagccacatccaaaatttatcaaatgtgcacgtgtgtgtgtgttgggggggccggtgcacagcacacgctgtgtccaccgggggtgcaggcttcggaggagacagaagggcagcagggacaactgtcacctttacaatccctcagatgctgacttcatgttcacagagcacataaagggaggggctgtggccagcctccacacccagcggcttgacgtcctccggggcctctgctcggagctgggcagccaggccgagggcccagggcgagcttgggggcctcactgagggtcggccttgcactgtcccatcaggccatcgccacacgggaggagctgcacctgcagcatgcccggggcctgcaggagaaggacgcactgcgcaagcaggtgcgggagctgggtgagaaggcagatgagctgcagctgcaggtgttccagcgtgaggcgcagctactggccgtggagggcaggctcaggcggcagcagctggagacgctcgtgctggtgggcctggggcctggggcagggaggggagccagcgggtgggacgcagggccgggggaacatgggccttcctgacccaaccctcctctgtgacgggcccccacctaaccgaaccccccctcgcagtggggtgagggcacaggagcagctcagggccccagcaggaaatggggctgcagggccagggtgggtgctgggcctggccatcaggcagcctagcacgttgctctgggcatggagggggcctggcgtggctaagggcatgcccagggctccctggaggatcccgctctgtgccctgcccaccctgtgcctggggagccctctgcccccacagcccacccaccccgttttctatgaccacagagctccgacctggaggatggctcacccaggagctcccaggaggtgagtgagaccccagccggcacgaggctggccttccttcacctggggtcctggctgagccgccaccaggcacagccccacactgcccgtcacgtggccctgctcagtgggctgggctcggtggaggaagagcaggtggccaaatactaaggcctccgacggctcaaggggtaaggcaaggcatggacagtgcccagctcctgcatcctcatggcgggaccttcctggctggcctcccccacatccgtctgctgagcacctgagtctctgagaagctctgtgcagggggtgatggcaacggccggggtgggcagtgtggccgggtccagtccttggcggggtgacctaacgggacatcttgcctctcttcggaagctctcgctcccccaggacctggaggacacccagctctcggacaaaggtaaggtggggacggcagtgcacctggaggcctcccggctgccccaagcgaggcccatcttcagcacaggcagcccgtgccgcaatgcctgtctcagcccccacaacccccaccccacagacggaaactagggctggggataagtaaaatggccaaggtcacccagagaacaaactacagagcccagtggaacccggggcccagctggtgggacaagtgtcccctggggtggagagtgtggggcccctctgggcagccccccggggtgcagaggtgagcaggcccactgtcctgagagtgtggggcccctctgggcagccccccggggtgcagaggtgagcaggcccactgtcctgagagtgtggggcccctctgggcagccccccggggtgcagaggtgagcagagggaggtggtgatgagcaaatgcccaggggtacaaagctagaaagtggggcaagacggggccccacgtggcttgagggacagggccgaatgcaggggtggccaagggcctgctgtgtcccggtgcagtgggggtaaggcagtggtgggggcttctagcgggcagggcaggccccctgtgcctctctgggggaagaactggagtggctggctgggagcagcccccgagtgcccaggagcggatagtggtgatgaggtaggtgtttgctctgaaggtggtgtcaggtgggagtcggggaggacccagcagaggggccctgggaggctgcgtgggagttggacgagcttcccgagcaccaggctgtggcctccaagcagaggcggaaggggagggaggagccgcagatggggaagtgaagaccagcacctttggccgcagcccagaacacttgaacttggggacgccaagcccccatgtaggcacagaggcgagcagacccaggagtccagctcaggcactgccaccatctccctgggtggctgtggcaggagtgggggcaggcatgaaggtccgagggtgagcaggggaggggctggctctaaggaggggttgccaatggccaggacagccctggcctggccttccccacagcggggctgagactggacatgcatggtttctgcccaagggtcctgacccctggtctgcccaggtcctggcgggttctgagggggtctagccacctgaaggttctctcccaattgtgcagtacccctgcccccgccactgtagctgtttccaactctctccccagggccgccatgcccacagatgctttggagtgatgggggacagtgctgctcaggccagggtttaagctcccctgaggtctgggaggccttcctgcctggggggcagccagcctcggggagggatggtcactggcaggagcgttgggcctgggctggcatctggcggcatcctggagcaatgaatgggcaagggaggccataatgcctccgtctcctctacagaaggcagagtctacactggacccatgtggggatcccaccaggcccacaaggtgtccgcaatgccggggaggggtggggcacccttgctgtacgtggccctgcaggtgggcagtccccacaggcacgactctcctttccaggatgcctggccggtgcggggagcccggaacagccctttgcagctctgcaccaggaacagctttcaccgaccccccatgtaaggcttcctcagggtggggtccccccagctgtgggcctcacggtggccgatcccagggagggtggctcctcgccctaggcaccccctgcagtggccccgacgacggctgaggggtgaccacaagccctcagtgcttggggtgcggttgtctggggcgcgaagtggatgcccctgttttcttgggtactgcagcagcttcggggggcttggaggtggacgtgcctggtcctggtttctttggggactttacagtggatgcggcgtcaaccctgagtaggtgaacacaggagtcctgagagcaaggactacctcgggctttgttgtagaaacaatggccaggagcccaggccggagcgtggtttggcctcctgcctgtgtcttggcatctgaaatgcaggctacccacaccggctcactccccgggtacaggcaggtcccaagcgcggcgagcttggcgctgagctgaggctgtctgagctccccgcctccccaccagtctgcagttaccggggccagtgggggtgcgagagagaagggagggtgccctcaggggagccggccggagcccaagcccttccccttctccaggacgcaggcctgagcagcggggagccgcctgagaaggagcggcggcgcctcaaggagagtttcgagaactaccgcaggtaggcgggcggcccccaggcttctccaaaacgggctggggagctgcagccgggtctgcgcctgagacacctgtgtcttcgcaggaagcgcgccctccggaagatgcagaaaggctggcggcagggggaggcggaccaggagaccaccggcagcgacaacacggacactgagggctcctagccgcagcagcgcaggccccgtccagggcacacccaccggcccggcctcccgccacctgggggtgccgacgccctggggcgcagacttcccccgagccgtcgctggcttagcctggaaggaggaatctggtgccctgaaaggctgagccgggcagcctggcgttgggggctgtttgttaagcggcactcagcttgaggaggccatgcgggtgctcgccacccccatgcacacgccatctgtgtaacgtcagggtctgttctatttcaccatgtaacacacaatacatgcattcattgtagaagtgtgagaaaacacggcagcttaaataaataaacagcacgggtgacccgca

SEQ ID NO: 4 - DNA, 3861 bp, macaca fascicularis
ggcagggtgctgggggcccagcactggggtcctcgaggcctcctgtaccctggctgccctcccagcagagaagcaggctttcgcccatttcatggaaagcacctgtggctttgttcttcatgctgagccggcaccctccccagatggctgccagccctcgcaccgccccagctgacccagggggttccctggaacaggtgacctggctacccagacacgatggcagcccccacaactttcactgtccttttgcgtgggcctttcttggcataagaccagcccccatcttctccgtctgtcctctcactccctttccttcgtctctgtggcagcagcgcccccaggccctttgctctctgggtttcactccctcactggaagcaccgtggggcctcagccctcggcacccggcggacaggacggtcccatggccggccgcgctcctgagcatgtgttgctgtgcctggttctctagcaactggtgcctagtctcctggtggccactctgacctgtgggttatttagtggtatattatttaatttccaaatatttagggattttccagatacctgttactgatttttagcttccgacacggttagagaacatggtatgtgtgttaattcctaaggacggacaccacagggaaatcactggggtccctcgttttacaagctggggaatcactgggtcatgagggcctggcgccagctcgggcccactccccagcagggcccaggacggcgttcctgcctgcacactcgctcatgctgctggtgaggggcccagcctggagtgtggcattcccgagagccccagaggacccctaactccactcctcactgcgtgatgtcaggcctgagggccgggggctgggaacggttaggcgagctccgcaccagcttcctggccttccagcctggcagcccccttctcgaagctgttgggtggcaactcggttcgacccgctttaccggtgaggcggggcctgggggctccccagcctgcagggatgaggctccagccgcccccacgccgaagtgcacagttcagggcagactctgctgcccgtcgtctgtggatggggggcgggctgtgccctgccatcaccggtgcttctgaagctggtttgggggaccctgggagtggcctcagtcctcacgtgcatcctggagaagagcaaggatgcctcgtgacaaggataatgatatcgagcgacacttctgaaagggacgcttctcccccgtgtgcagctgggtggtggccggtgccatggcccctggcaggctgcagtggcctcctgggctccataaggtgggctgcagccctttcatgaccaaggcgtgtgctgggcatgctgtgtggctgtgtgcccccctgcacatgccctgccccgctgtcctgagatgccggcgagggcaagggtgtccgcacagggcagagcatgccctcctgaagaagcctccagaaacctatgaccaggtcaaagcggctgggtgcggtctggcctcgtccagcctcctgggggtctctgctgtctcttctacttgccaggtgcccgggctgggcctctggggcattctcaggacccctggcccccggccaggctgggagggctgagagcagttttcctgtggagggcagaggacttccacttccgtctgcctctcagggcctcctgggctgcacgaatcagaaggcaggccaccagggactgtccccctgtgggaggactggcgggactgtggagttaagccatcaggaagtgcacaggcgtccggcacgctcctccctccctgcagccccaggcagcatctccccgaggctccgcgtcccaggctcctggtgcgtctgccgtgcaggtggctccttggaagaccctcagcctgcctgcgaggccatgtcggactacgagaacgacgatgagtgctggagcgtcctggagggcttccgggtgatgctcacgtcggtcatcgacccctcgcgcatcacgccctacctgcggcagtgcaaggtcctgaaccccgacgatgaggagcaggtgctcagtgaccccaacttggtcatccgcaaacggaaagtgggcgtgctcttggacatcctgcagcggaccggccacaagggctacgtggccttcctcgagagcctggaactctattacccacagctgtacaagaaggtcacgggcaaggagccggcccgtgtcttctccatgatcatcgacgcgtccggggagtcaggcctgactcagctgctgatgaccgaggtcatgaagctgcagaagaaggtgcaggacctgaccgcgctgctgagctccaaggatgacttcatcaaggagctgcgggtgaaggacagcctgctgcgcaagcaccaggagcgggtgcagaggctcaaggaggagtgcgaggccggcagccgtgagctcaagcgctgcaaggaggagaactacgacctggccatgcgcctggcacgccagagcgaggagaagggtgccgcgctcatgcggaaccgtgacctgcagctggagattgaccggctcaagcacagcctcatgaaggctgaggacgactgcaaggtggagcgcaagcacacgctgaagctgaggcacgccatggagcagaggcccagccaggagctgctgtgggagctgcagcagcagaaggccctgctccaggcccgggtgcaggagctggaggcctccgtccaggaggggaagctggacaggagcagcccctacatccaggtactggaagaggactggcggcaggcactgcgggaccaccaggagcaggccaacaccatcttctccctgcgcaaggacctccgccagggcgaggcccgacgcctccggtgcatggaggagaaggagatgttcgagctgcagtgcctggccctgcgtaaagactccaagatgtacaaggaccgcattgaggccatcctgctgcagatggaggaggttgccattgaacgggaccaggccatcgccacacgggaggagctgcacctgcagcatgcccggggcctgcaggagaaggacgcactgcgcaagcaggtgcgggagctgggtgagaaggcagatgagctgcagctgcaggtgttccagcgtgaggcgcagctactggccgtggagggcaggctcaggcggcagcagctggagacgctcgtgctgagctccgacctggaggatggctcacccaggagctcccaggagctctcgctcccccaggacctggaggacacccagctctcggacaaaggatgcctggccggtgcggggagcccggaacagccctttgcagctctgcaccaggaacagctttcaccgaccccccatgacgcaggcctgagcagcggggagccgcctgagaaggagcggcggcgcctcaaggagagtttcgagaactaccgcaggaagcgcgccctccggaagatgcagaaaggctggcggcagggggaggcggaccaggagaccaccggcagcgacaacacggacactgagggctcctagccgcagcagcgcaggccccgtccagggcacacccaccggcccggcctcccgccacctgggggtgccgacgccctggggcgcagacttcccccgagccgtcgctggcttagcctggaaggaggaatctggtgccctgaaaggctgagccgggcagcctggcgttgggggctgtttgttaagcggcactcagcttgaggaggccatgcgggtgctcgccacccccatgcacacgccatctgtgtaacgtcagggtctgttctatttcaccatgtaacacacaatacatgcattcattgtagaagtgtgagaaaacacggcagcttaaataaataaacagcacgggtgacccgca

SEQ ID NO: 5 - DNA, 9251 bp, mus musculus
caaagaggactcctgactgctgcttttggaagtattttcaaggccactgtccccataggagcaagcagagggccagtaagctacagaaggggccagctggggctctgcataagtgtcccacccttccttcccatacagcactggaagcttttgcctcaactcatcttgtaggctagacagcagcgcctgtctaacagctacactggctgggcagggctagcaggtccccacgcccacctcaggctgactcttgaagggccaccctgaaggagaggctgtggcacgcaatccatagaggactatagctgcctacagcttgtagattttgaccaagctttgtcccactgcacctaccctgcaccactgtccttagacggcagatgaggatatgaggcagggccatgttagaaaagcaactttcagaagcctggatccaggaggaccttggactgcccatccagttcagatgagcaggcagggagggctcctctgctcccagagtacttctacttccctctgttctgttcctggatgggccaatcagaaggtagactgtcccctgcgggaggactggctggcttgtggcgtcaaccagaacacatcaatcaggaagtgcacagcctccttcatggctccacccttctccagttagggaacccctccacactcccagagacccaggctcctggtatgtccataacccagacagcatctgctggcaggtagctctcacaagaccctgaggtaagttcagtacccttccaggaaggtgggtggaagcctgtggtgactgctggggatcctggagcgcgtaagtacctacaggcaggtggcatcctccatcctcacgccttcatttgattaatccttgtacactgcagcatagtcctccagtgctggaccatgtggctgtggggctgcaatggcaggccataggcccaaggtttagagaaatgtctgtccactgggtcaaccagaaagcagcaggaagccaagagatgcctctgaagaagaggcctgccatggccgtgtctctaactggaggtgcagctccgctggggttagggactgagccaggggcgctctatggccaccttaactcagccctaaaatcattgaagccattcactgaggctacacttgtccacatgagtaaccccctacatacaactggcctggttgattcccatcatgtggccaaaggccagggctcatctagcggcagaaaagcaaactggagggaagccacagcagatgtctgtagactgtggatgacattgcctgcccttccagtatactccagcctagcaggaaggcagacatgctcctgcggccaaccctcccagccctcccggaatagcaccgtgccacaggctgttgcatcaccagctggctacaacggaaggcctgaagggcacggggctgtcagggaaggaggaagtcagacataggcctggccccctgcacagtgagaaagcctccagccaacctgaccctcttagtcccaatctggagactggccagcaagggtccatgccacccaagaaggtactatgtaggatggccactagaggcaaaagctcctacctccagggcatgccagagcacttttgggtaggggcagaagctagagcagggctgttgtacaaagagtattctcttagaccccaaccggtcacttccctacatatccatatgagaaagcctgagatgacaggggccgggcagctgcttgggggtgctcagtttgacaggtggtctgaggtggcttgtctatggacctctgcttcccctcagaaactgtccatgaggctgaggagatagggctgagccctgaaatttccctagaacggatcccttctgaccctgcccaaagccctgccctgacactcaggcctgggtcctatcctaggctgacagggaacagaaggtgggcacttcctcagctgctctccccaattataaccgcagcctacagaggacatgtcagactatgagaatgacgacgagtgctggagcaccctggagagcttccgggtgaagctcatctctgtcattgacccctcccggatcacaccctatctacgccagtgcaaagtcctgaaccccgatgatgaggagcaggtgctcagtgaccccaacctggtcatccgcaagcggaaagtgggtgaggactgctcccacaccagcaggctctagccctgcatgacccccataagacacccagccagaggccactccatgctctcttcactgccctggccttgtccccactcagctccatccccgtgtaggatatatgagtgagagactcctcccctcttgtcctgtctacctgctgttgcccagtggaggaaggggagtccccaccactgttcaggccctgcctgagacagccaccagagggaaggcagcaccctgtcccacctagacttgagacctgcagggcgccttattcaatggcccagaaggaagggggttgctagagcttgggatgtctacccagagtaatcatcctacccatctccaagagcctccacccgccagcccttggccttgactctgtcttccacctgaaggtgtgctcctggacatcctgcagcggacaggccacaagggctacgtggctttcctcgagagcctggagctctactaccctcagttataccggaaagtcactggcaaggagccagcacgcgtcttctccatgatcattggtgagaggcacgggtgtgggttgttgctgtgggtggactcgggtcctggcttctatgatggcgttggagtgactaagtgtgataacaagccttacgatgtagatcagggtagtggtccctgatgatactcacaggctctagggggcagggtcttcctgaattgctgagctcttactggtctctagaaggggagcccttctctgcctttcctactcacagcctggaaatttcactgagaagtagtgacccctctgaagtgccatttctgcagggcccagttgtcagccaagagcagaatcctacagttcccagaggggacagaggtgtcctctcctgatggaagagccacaaccccatcaattctcattcatctcataaagagcactgtgagataaacagggcaggggcgatggcacacacctttagccctggtagtctggaggcagaggccaacagatctcttgagtgagaggccagactggactacatagcaagacgttgtctcaaaaaccaggggctagggggagaagagggtcaactggagtcttcacccagggcatgtgagtggccctgggtgatgagtcagaatctcagttcacctgtgagggacagcaaggccaactgggcctactgagcacactcagtcttgagagacccatcctaccagttttctctgtgggaggccaggaatgctccctctgtcccccctagtcttcaaatgcacacttacggacaaggaccagcacggggtctgcaggtgacaaccgcaggctccatctgacaccgcatggtccccagatgcatcaggggagtctggcctgacgcagctgctgatgacagaggtcatgaagctgcagaagaaggttcaggacctgacggcccttctgagctccaaggacgacttcatcaaggagctgagggtaaaggacagcctactgcgcaagcaccaggagcgggtgcagcggctcaaggaggagtgtgagctgagcagtgcggagctgaagcgctgcaaggacgagaactatgagctggccatgtgcctggcacatctgagtgaagagaagggcgcagcactcatgcggaaccgtgacctgcagcttgaggtaatcactttctccacctgcagggggtggcctggaggaggctgcagctgactgggccacccacaggtggaccggctcaggcacagcctcatgaaggccgaggatgactgcaaggtggagcgcaaacacacactgaagctcaggcacgccatggagcagcggcctagtcaggagctgctgtgggaactacagcaggaaaaggacttgctgcaggcccgggtgcaggagctgcaggtctctgtgcaggtagggagacccaccagtgcacatcaggcctcatgcctgcaggggcttgaagctgggcacaccagatggtacaactgcagtggccgtgccattgtgggcctcctcagccaggcttgttgccttctgacacctccaggagggtaagctagacaggaatagtccatacattcaagtgctggaggaggactggcgtcaagcactgcaggaacaccagaagcaggtcagcaccatcttctccctacggaaggacctccgccaggctgagaccctccgggcccgggtgagagaccagggcagacaagcaatgcccactgggatcctggatggtggggtctgggacctggctcactgtcaattcgtacagtacacagtagtgccgtgtcaccctttgggcttacagtgtgaccaatggtggctgaggccatggtcttagactaggttagtcttggattagattggactggaactccagtgccttcctcattatcccagaccttgtgtgccctggagctgttgctctgagtccagcccagggggaggaggagggaggggagggtgaggctgagtggtgcttcctgtagtgcacggaagaaaaggagatgttcgagctgcagtgcctggccttgcgcaaggatgccaagatgtacaaggaccgtatcgaggctatcctgctgcagatggaggaggtctccattgagagggaccaggtaggtgccacacttgtgttcagaaacggagaaccaggagtgttggggtattggtgcggcagacactggagcagtagtcaaagtgttttgttttgttttgttttttttatttatgctcagggtaacttggcagtgttggaagacagaacccgggactgcttgtacaaatgcatcaaagtctaccttattcaaaggaggagcttatgcgggaggagaacacagcagtccctgtggtgggggtgggggtggggtaaggaggtgggagacttcagtagggagaggaagctgctaggtgcataggctgtgggtagggagcatttggtacaagaacctgtggtggctcccgggcagaggcagtagtagcaactcacacctgcaatcccagcagttggaggttgagacaaaataattactgtgaatttaagggcagcctgatttaccatgaattccaagccagtttggggtccaatgtgagaccctgtctcaaaaagccaccactaacaataacaaaagcggtagcagctgagccatgtttggtagtacatggcagtcccagcatgctggtccccggatttagaggtaaagacaggagagtcagagattcaaggccaacctcagctaacagtaaataggaggccaccttgggataatgaaactgtcttccaagaaacaaaagaaaaaaacctttgtgttcatttaagaacagccatgaatcctggtttgtttgtttttttttttgtttgtttgtttttgttttgttttttaatgtcaagctgcatccagaatttataaaatatgtcacataggtgggacagaaacacccactggctatctgctggggatgtaggacttctcagttgggcttcctatgaactactgagcattaatcacgggtttttttggctgcttctctgaagtgttttgttttgttgtctgagacagtgtctcatgtaatccaatcttgccttcaactcaatatatgtgcaaagatggccttaaactattgatcccacttctgcctccccagtgctaagaatctggtcatttccaactccgcccagggtttgttcatgtacactgagagccctgactgctttgagaatccctccagttcagcagttccagccatacaccagggtggttgatgtccagccaggacctgaactgccaaatgagagccaagagcatcaaccaggtcctttgctgagggtgtgccctctgctattcctacaggctatggcctccagggaagagctgcatgcacagtgtacccaaagctttcaggacaaagataagcttcgaaagctggttcgagagctgggtgagaaggcagatgagctgcagctacagctgttccagacggagagccgattactggccgccgagggcagactcaagcagcagcaattggacatgctcatcctggtaggcctctgtgtctgctgtcgccagggtgggctgggcagtgagagagctggggaggatgtgaagtcaaggcaggattccaagaggaaccatcagcctggaagcagccctatcactaacctgccatgactgctgccctgcttgtggcctcggaaaatgcctggccttcctaaatcccagttttcctcattagagctcttcatggcgaagtgacagccacagatgtaactcaaggttatccacaccactccaatgactgcagccttgaccaaggagtaaccttcaggtggcttggagtgtagtgttaagaaagacatattctgggcatatctagaaacatgtgcagggcctccctagaggatggagcctggatggggctcttaccatgccctacacaccccatacccaggtgcttggaacccctacagcccactgaccaactcccttttctgtggccacagagctctgacttggaagacagttcacccaggaactcccaggaggtgagtgtggacatagagtggtccttccctggccttcaacctctacctgacctcacactcatctcccacatggtcctgtccccttaaactgggctgtcccttttctctcattttgggttggattcagggaagagtgctggccaaactctaagctgttgctgtgagcgaagtgagaagtgagtcatagactaatgtcccatatcctggcctctcaggacaagagcttcatgctagggtttcctttgcctgttggcctgactgggacggagtccctgagaatctctgtgcagaggggtgatagtgatggctgtgtcaactgaggagctggagtcaagcgggcagtgggctgggccacacttagtacaggaagggcaatggtggcttgtagcatggtctaactgggtggcgtgtctctgtttggaagctctcactgcctcaggatctggaggaggatgcccagctctcagacaaaggtgaggataccgtgtactatcagaggcatgtaggctgcagtctcctggcctccctaaccctggcccatcctcagcacgtaaagctgtgtcacttactttgtctcagaattcacaccactccagccaaggatgatactccccaaggcaaggagagtcaatggtgaagggcagaactcttagactaacacacccggagaataaggaatctgatttgaatgtgggacttaggtctcagggtctgtggctcagaggacagtgattgtggaggccccatccctcataggcccatctttccccactagccttcctcctgcagggggagatactagccagtacccaggtgtgctcaatgaggaggggaggaatagagctacggacagggccacaagcaaggagctgggggttgtgctatccacagcaggctgacaggcgggaccctcctctgatctaatagctggggggtggcagctggggcagctgagggtgggtggcgagagggtctggcgcagggaggagcccagggagctttcgtgacaagtcagacagacttcctcagctcagccttaccccaggctctaaccaaaggcaaaaggggaacaagaggtgtagatggagaagcacgggtcacccactgcagcccccaagacccaacctgaggtcactgagctccatgtgggcacacaaggagaaggatgcaagagcagccagctcccttgaaggcatgccaacagttaggcacaacgaaaatgagaggggaagggggcagggctgctgggacggggttgttctcacaccctgagccctccttggatataattgtttcttgatgttcatagccttccctgcacatcttctacctagaggcttgagccttggtttgcccaggacctggggcatgctggctctggctacctggaatgattattcttctaattaatatggtgtacttgcactaacccaggcccccgatgccctgagctctccactgaggctgcccccacaccacccatgggaaccactagacacaaaaatgttgccaagctaaaagggacatggactggggcatgcattcgcaaataatcatggggacctggagtctttcctagccatctgtccttgtgtttggtgacagtcagcttgggcggtatgggtgactgatggtaggagccctgttgatgtagcagtcaagggctcagcctgtttctgtgaaatgcagtccccactggactcatgcaaaggtcccagcccgtaaggctgacctaggctaatgctagttgggctgtttgcagagggaggtggccataccagatggggcccccataagcacagatcttcctttcaggtgtactggcagacagggagagcccagagcagccctttatggctctgaacaaggagcatctttcactgacccatgtaaggctgattggggttcccaagagccctggttactacatggctgatcctggggaggagggtgacgccactggacagctggggatcacaaggtctttggtagtgtatgggtaggatgtaacatttggatagtggggtggttggacaggggaggggacaatgtaaggacacccatactgctgttcactgcaaagaagtcttccactcacctggctacaaaggggttgtcaccccacgctcagtcatctcggagtccagcagaccccacggccagctagctatgctccacacctgctggagtcccagggctgccgtggtaaggccacggtgtccagctgcccgccccgttgtccagctgccggccccgttccttctctagggcatggggcccagcagcagcgagcccccggagaaggagcggcggcgcctcaaggagagcttcgagaactaccgcaggtgtggggcggcgggcaggacggctggcgggtgggacacctgtgcctacctgctcacactgcgggccccacaggaagcgggcgctccgcaagatgcagaacagctggcggcagggagaaggggatcgcgggaatacgacaggcagcgacaacaccgacaccgagggctcctagcgaaccgcgccgaggctgagcatctgtggaat

SEQ ID NO: 6 - DNA, 2551 bp, mus musculus
caaagaggactcctgactgctgcttttggaagtattttcaaggccactgtccccataggagcaagcagagggccagtaagctacagaaggggccagctggggctctgcataagtgtcccacccttccttcccatacagcactggaagcttttgcctcaactcatcttgtaggctagacagcagcgcctgtctaacagctacactggctgggcagggctagcaggtccccacgcccacctcaggctgactcttgaagggccaccctgaaggagaggctgtggcacgcaatccatagaggactatagctgcctacagcttgtagattttgaccaagctttgtcccactgcacctaccctgcaccactgtccttagacggcagatgaggatatgaggcagggccatgttagaaaagcaactttcagaagcctggatccaggaggaccttggactgcccatccagttcagatgagcaggcagggagggctcctctgctcccagagtacttctacttccctctgttctgttcctggatgggccaatcagaaggtagactgtcccctgcgggaggactggctggcttgtggcgtcaaccagaacacatcaatcaggaagtgcacagcctccttcatggctccacccttctccagttagggaacccctccacactcccagagacccaggctcctggtatgtccataacccagacagcatctgctggcaggtagctctcacaagaccctgagcctacagaggacatgtcagactatgagaatgacgacgagtgctggagcaccctggagagcttccgggtgaagctcatctctgtcattgacccctcccggatcacaccctatctacgccagtgcaaagtcctgaaccccgatgatgaggagcaggtgctcagtgaccccaacctggtcatccgcaagcggaaagtgggtgtgctcctggacatcctgcagcggacaggccacaagggctacgtggctttcctcgagagcctggagctctactaccctcagttataccggaaagtcactggcaaggagccagcacgcgtcttctccatgatcattgatgcatcaggggagtctggcctgacgcagctgctgatgacagaggtcatgaagctgcagaagaaggttcaggacctgacggcccttctgagctccaaggacgacttcatcaaggagctgagggtaaaggacagcctactgcgcaagcaccaggagcgggtgcagcggctcaaggaggagtgtgagctgagcagtgcggagctgaagcgctgcaaggacgagaactatgagctggccatgtgcctggcacatctgagtgaagagaagggcgcagcactcatgcggaaccgtgacctgcagcttgaggtggaccggctcaggcacagcctcatgaaggccgaggatgactgcaaggtggagcgcaaacacacactgaagctcaggcacgccatggagcagcggcctagtcaggagctgctgtgggaactacagcaggaaaaggacttgctgcaggcccgggtgcaggagctgcaggtctctgtgcaggagggtaagctagacaggaatagtccatacattcaagtgctggaggaggactggcgtcaagcactgcaggaacaccagaagcaggtcagcaccatcttctccctacggaaggacctccgccaggctgagaccctccgggcccggtgcacggaagaaaaggagatgttcgagctgcagtgcctggccttgcgcaaggatgccaagatgtacaaggaccgtatcgaggctatcctgctgcagatggaggaggtctccattgagagggaccaggctatggcctccagggaagagctgcatgcacagtgtacccaaagctttcaggacaaagataagcttcgaaagctggttcgagagctgggtgagaaggcagatgagctgcagctacagctgttccagacggagagccgattactggccgccgagggcagactcaagcagcagcaattggacatgctcatcctgagctctgacttggaagacagttcacccaggaactcccaggagctctcactgcctcaggatctggaggaggatgcccagctctcagacaaaggtgaggataccgtgtactatcagaggcatgtaggctgcagtctcctggcctccctaaccctggcccatcctcagcacgtaaagctgtgtcacttactttgtctcagaattcacaccactccagccaaggatgatactccccaaggcaaggagagtcaatggtgaagggcagaactcttagactaacacacccggagaataaggaatctgatttgaatgtgggacttaggtctcagggtctgtggctcagaggacagtgattgtggaggccccatccctcataggcccatctttccccactagccttcctcctgcagggggagatactagccagtacccaggtgtgctcaatgaggaggggaggaatagagctacggacagggccacaagcaaggagctgggggttgtgctatccacagcaggctgacaggcggga

SEQ ID NO: 7 - DNA, 8134 bp, sus scrofa
aggccgccctgctcgggcgaatcagcagcggccgcagggatggtccctgcggggttaacgccaggaagtgcgctccctcccgccccaccctccctggccggcagccgcggcagctcccaccgctcacacaccggcttggcggcccaggctggccggcgaccggccactgcacaggtgcctcaggaggaccccaaggtaggctggtctggtgccctttcggaaaggcggcggcaagcccaggtggctcaggactcttgggacagggagagggatctggcgtgaacaccccagaagggcaccaaacggcctcacctctgctgccccgtccccctcgcagccacctggcacggtcgcttcggtggaatgcacagcaggtcatcgtcaggcgtgggtgctgtggacttgaggctagaaaggccgcaacaggccaggtccagagaccggccaccaaggtgctggtgattcctctggacgccgcgcctgtcgctaggcgggcgggccgagggcggtgggacccacagccacggcctctgggataccacggcctctgggaccgtgccccagcccccgtggcccgggagccggtgtttcccaggcaggttacaggctcagtcccaggtgttgaggcggggtctctgtgccccccgacccctctcccccaggaggaggcatggccacatggctgctcctgctcagcccgccccagagcgtggtccctgcccacccgctgctggaagggctagcagagagcagcttccacgtagatggtcccctgtcagggagccagcatccccgtgctcagcactgtggccgaggagatggggagagcccggggccagctagtgcccagcacccagagcctggaggaggcgcgcagccgcgtcctgggctgcctgggcgacacagctgggccctccccatgtgcccagggccctccggtgggctgctctcagtcacgcttcctggagggcaccacgtccctgggcctgaccggagcgggccagctgggcaactgttgcatcaccagccacaggaagcagaggaagggatgggagtggatgtgcgggggaatgaggaagtcccgcgtggcatggcctgccgcccgcaggtggggggcgccccagccgggcgcgggctggccgggggtggaccgggacccctccctcatctgccggggaggttgtgccagcgctcggcccaaacagctcctgctgccccctcacctccagggagggaggcagcctccctgcccgcccacaatgctcggggagggggattctccctgggcacaggctgggggggtagcgtggggcaggggcccagggtgacgggggcgggggagaggaagcccagagcggccagagggcgggggcagggcagggcagggcggcgggccagttgacgggccctctctcggagcagtgcgcccgcccgcaccatgtccgactacgagaacgaggacgagtgctggagcggcctggagggcttccgcgtgaagctcatctccgtcatcgaccccgcccgcatcacgccctacctgcgccagtgcaaggtcctgagccccgacgacgaggagcaggtgctcagtgaccccagcctggtcatccgcaagcggaaagtgggtcagtgccgtgggcggcgggctctgcccacgctcgtccctgtccccgggaggcctggccggctcggcccccgccccgccccccccatcaccaacctgtctgcctgcggccggggcgccgccatgcacagcagaggcccctgggagagccacacgaggtgggtggccgatggggactggcccagacccactctgcggccaccccgcggggcctcgaggggccacacgcgtgcctggggacgccggccgccccggcccccagccaggcccatctgggttgcaggcgtgctcctggacatcctgcaacgcaccggccacaagggctacgtggccttcctcgagagcctggagctctactacccgcagctctacaagaaggtcacgggcaaggagcccacccgcgtcttctccgtcatcattggtgaggaagggcaccgggcctggggacccagggcccggactcaggggccggggcgcccccctgacctgggcccctaacggataaagcgtgcatgcgctctggcggaaggataccgcccgcctggggcccgcccgcccgctctcctgctgcagcccggggagggggcaggggccggagggaagggggacctgaactccagggcgggagcagggggaggggaggcacaggctgattcttggctcagagcccccctgcccagaagaccacctgaccccactaaaaccctgctcccgagaggccgcggcagccggagaggtgccgctctcctggtcctgagtcgtgtcccctcgcctctcaatcaagatgaccaaagtggacaaagagggagggacacgtgagcccctggctgcaccgtcctgggaggggctggggacggggcctgcaggtgagtcaggaccagacccctgggccgggctccccgggccgccggtcctggagccgcgcgagatgctgggctgctgggcgcgccgtctgatgaggcccggtccgcagacgcgtctggggagtccggcctgacgcagctgctgatgagcgaggtgatgaagctgcagaagaaggcgcaggacctgacggcgctgctgggctccaaggacgacctcatcaaggagctgcgcgtgaaggacggcctgctgcgcaagcaccaggagcgcgcgcagaggctcaaggaggcctgcgaggccggcggccgcgagctccagcgctgcaaggacgagaactacgacctggccctgcgcctggcccgtcagagcgaggagaggggcaccgcgctcatgcgcaaccgagacctgcagctggaggtgcctggcggcggggcggccgggcgggcggcgggcgggcggcgggcccccgggctgggctgacgggcggccccgcagatcgatcggctcaagcacagcctcatgaaggccgaggacgactgctcggtggagcgcaagcacacgctgaagctccggcacgccatggagcagcggcccagccaggagctgctgtgggagctgcagcaggagaaggcgctgctgcaggcccgggtgcagggcctggaggcctccgtgcaggtggggccagcggggcgagcacagcgcagccgggcggggggcctccagccccgccgccccgccaccctctcccttggcgccacaggaggggcggccggaccagagcagcccctacatccaggtgctggaggaggactggcggcaggcgcagcgggacctccaggagcagacccgcaccgtgttctccctgcgcaaggacctgcgccaggccgaggcccagcgcgcccgggtacgcggcccgcggtgggggaggtgggctggcctctcagcgctgcgtccctcccggccccttcctgcaggcgaggcccggctggccggccggccccgccccgagcgcgtgctccccaggctgggtccagggtccgggccccgggccggggttggcggggctgcgggcggcggggttagctgccgtctcgcccccgcagtgcctggaggagaaggaggtgttcgagctgcagtgcctggcccttcggaaggactccaagatgtacaaggaccgcatcgaggccatcctgcggcagatggaggaggtcgccgtcgagcgggaccaggtagcggtcagggcagagcccctccggggaggcggcggtcaaacaggctttacacaacgcaagcaggtcacgctttacacacggttctcgccgaagaaggagagacgttccccggtggcgcggcagcaggttaaagacccagcgttgccactgccatggctcaggtggatgctgcggcatgggcttggtccctggcccgggcactgcccgcagccgcggccaaaaataaactgagaatgctgaggacgctaatccccgcgccaccttccgctgtacttgccgtttcttcttcgctctacaagaatgggagttttctgacttgttttgcaaatggttttttagcttaagaagccgtggcgcctgtgtgggctgccctcttgtgtgcgggagcatgcccggcgcagggtggcgtctgctccctgccccatcgctctcctttctaatcggtcacagccgatgttccagtcgctttctggagggtgactgtcacttggtgtcaggagccttaaaacgcagattccttttagcccagcaatccgcttctagaagcctctctcatgggaaccacagccgtgctgaaagcccggtaggtgggttcttatctcaaggccatggacacagtgagatcgggggcggcacctggaaatgggcagctcagcagacaggaggtgcccccacccaccgcagggagtgcagggctgtggctgggcccgggaggccctcggcctggggaggctggggcctggccgcccaggtgagtcagggtggggggtctgtttggtcctaagctggcagcatgcacagaaatgaaggcagtgctctgactaatcaggccttgggagtgcccgtcgtggcgcagcagtggtgaatccagctaggatccatgaggacgcgggttcgatccctgcagcgtagctgtggctgtggcgaaggccggcagatgcagctcggattcatcctctggcctgggaacctccgtatgccgcaggtgcggccgcaaaacagatcaagccctggccgtttggggtcgcctgacacaggggatgtggttgggctccgtgaattgcggctggagacgagactggcttgtcctcgggtgaccaggggcgtgggggtgcgatgagagaggtggttaaatcccacagccgtcaaccgtcgtgctcctgagctcaagccccacccagggtgtgtgacccccgcgtgtggggaggcgagcgtgtgccgagcgtgccagggagcggttggcaggggctagtcctgagggtcattgtctgtagattttctgaacatacacgcgttctgtcacgcgcctgtatcacgtttacgaccccaaacacgaaggacgtgcttgaggacagaggaagaagccagtgccggccccccgcccgccctcctcgcgggaacacggctagagatggggcggcctgtgctgcgcttgcggggtccccaccgcctgccccgccctgcgctgccgccccacaggccatcgtgacgcgggaggagctgcacgcgcagcaggcccgcagcctgcaggagaaggacgtgctgcggaagcaggtccgcgagctgagcgagaaggtggacgagctgcagctccagctgttccagcgcgagggccagctgctggctctggaggacaggctcaggcggcagcagctggatacacctgtcctggtgggcctccctggggtcgggggaccctcggtcagagccacgcctgtgcctccgtggggcccggggctgtgtccactctggggcaggtgtgtccctagagaccgcagaggcccgaggaggagccgccggtccccagcccttcctgagccacctccctcctgctggtgggctcagggcctggcaggctgctctgggtgctgtgatggggacagagaggggctctggccaggggcctccctggagggtgggctggtctgggacccttctccacgcccacccccgcaagcctgctgactgacgcctcttctgtgaccccagagctctgacctggaggacagctctccccggagctcccaggaggtgagtatggccccgggggtcagcccagggccggcctcgcgcggggctccgggcctcaacctccgccagccaccctcccggtgctgggcagagcagcgcgggcccctgagaagcttgcaccggggccgggggtggggcagtgacggggaggggcacacagagggctcggccgtgcggtggggggtgctggtgtaaccgggtgcccatctctcctgggaagctctcgctcccgcgggacctggaggaggacgcccagctctcagacagaggtgaggcggggagccgtctggcgaggaacctggccagagcctcttgatctgctcaagttggcgcccaccctgaccttcatcacacaaccgaggacgcggctgccccaggacacgcgccccagccctgtcccgctggccttcaccgccccgctttacagatggggacaccgagaagcagggggataagcaccccccacggttcccagcaagcaaacaagagccccatttgaacccagggtccatgtttgcaggtccaaatcccagctccgtccttgtgggcacgcctggccctcagagcccactcgagccccgaaggcagagtggcggcacctgccccctggagcagggccctggggaggtggcagtgggtcctgcccgggtgtgccatgccagggagggtgcacagcatgcgggaggggggacacaggtggatggcactgggggcttcgtgtgcgggaacataggacacagggaggcagagagggtggtgacaggggctcgctctgaaggcgggtgtcagatgggggtttttgaggactgggcggggagggggcgggggcggcgccgcccgcagaggtagtgcgtgaggccggacagacttcctcagcggcaggcttgaccccagcctgggggtggggatgcgggcaaaaggggaagaagaggcagcaaatggagaagtaagggctcggtccctctgctgctgcccaaagacctgagcttgaggccaccgagcccccacggggagaggcaggcagcgccttcaggcagccagcggcaccctggggttcccagcccaggctgctgtggcccctggcgggaggccgggtagccttcaaggccagagggaaaggtggcagcttcgggagagacagggggctgggcctctcccacggcgatgtccctgggacctggaacccacaggccccaataataaccagtttagggggtcagcccatcagagcaccgtttccaagagtcctgagccctgggcccgtccgggcgctggcaggtttgggagagtctgcttgggagcgctggaatctgctcctggaagcgaggaggggccgtgaggtccccgagccccagtcctcttgtctgtgcaatgggagtccaagcatgcacctggcaaagggcacaggactggctcagggtgaggaggtggaggtgccgtgggcgcgtcctggcaggtgggcaggaccaacaggcacagctctcctttccaggtggcccggccattgaggagagtctggagcagcagaaggagcggctttcgctgacccccaccgtgaggctgcctggggacaccgggacacccggggggtgatgacaaggctctctgccctggggcccctggggtgcagtgtttggaagagaaggcagggtgagggccaggccggggctctggccgcccctgccccctgcccgccccggaagcctgtctcctgggccgggtgcagcgctaacctgagtgggaacttggcctctctgtccaccacacggagcctgacagcaaaggggctgcagggacactgcagaaggaacactggccgggagccggcctcactggctgtgcaccccggcccagccttggcatctgaaaggcgggacagcccaccctgacccccccctggtccccagttcgcagccccaggggtggaggtggggtgcctgcggaagcctgagcctcacccctcctccaggacgcaggcctgggccgcggggagcccccggagaaggagaaggggcggcggcgcctcaaggagagcttcgagaactaccgcaggtgggcccccgcgagcagtgggcagcaggggcaggggcacgcctgcgcacacccgtgcacatccgcgccgtctgggtctcccgcaggaagcgggccctcaggaagatgcagcacggctcgaggcagggggaggtggactgggagaacaccacgggcagcgacaacaccgacaccgagggcccctagccggcccctccgtccagccgcctgggacctggccgctgccccgctaggccgcctttgttgacgagcacgggcgtgttctgtctctggggaatgcacacgcacgttgcagtgttaaaaacgcagccgcgtcaataaacgagcaccgtccgga

SEQ ID NO: 8 - DNA, 1967 bp, sus scrofa
aggccgccctgctcgggcgaatcagcagcggccgcagggatggtccctgcggggttaacgccaggaagtgcgctccctcccgccccaccctccctggccggcagccgcggcagctcccaccgctcacacaccggcttggcggcccaggctggccggcgaccggccactgcacaggtgcctcaggaggaccccaagtgcgcccgcccgcaccatgtccgactacgagaacgaggacgagtgctggagcggcctggagggcttccgcgtgaagctcatctccgtcatcgaccccgcccgcatcacgccctacctgcgccagtgcaaggtcctgagccccgacgacgaggagcaggtgctcagtgaccccagcctggtcatccgcaagcggaaagtgggcgtgctcctggacatcctgcaacgcaccggccacaagggctacgtggccttcctcgagagcctggagctctactacccgcagctctacaagaaggtcacgggcaaggagcccacccgcgtcttctccgtcatcattgacgcgtctggggagtccggcctgacgcagctgctgatgagcgaggtgatgaagctgcagaagaaggcgcaggacctgacggcgctgctgggctccaaggacgacctcatcaaggagctgcgcgtgaaggacggcctgctgcgcaagcaccaggagcgcgcgcagaggctcaaggaggcctgcgaggccggcggccgcgagctccagcgctgcaaggacgagaactacgacctggccctgcgcctggcccgtcagagcgaggagaggggcaccgcgctcatgcgcaaccgagacctgcagctggagatcgatcggctcaagcacagcctcatgaaggccgaggacgactgctcggtggagcgcaagcacacgctgaagctccggcacgccatggagcagcggcccagccaggagctgctgtgggagctgcagcaggagaaggcgctgctgcaggcccgggtgcagggcctggaggcctccgtgcaggaggggcggccggaccagagcagcccctacatccaggtgctggaggaggactggcggcaggcgcagcgggacctccaggagcagacccgcaccgtgttctccctgcgcaaggacctgcgccaggccgaggcccagcgcgcccggtgcctggaggagaaggaggtgttcgagctgcagtgcctggcccttcggaaggactccaagatgtacaaggaccgcatcgaggccatcctgcggcagatggaggaggtcgccgtcgagcgggaccaggccatcgtgacgcgggaggagctgcacgcgcagcaggcccgcagcctgcaggagaaggacgtgctgcggaagcaggtccgcgagctgagcgagaaggtggacgagctgcagctccagctgttccagcgcgagggccagctgctggctctggaggacaggctcaggcggcagcagctggatacacctgtcctgagctctgacctggaggacagctctccccggagctcccaggagctctcgctcccgcgggacctggaggaggacgcccagctctcagacagaggtggcccggccattgaggagagtctggagcagcagaaggagcggctttcgctgacccccaccgacgcaggcctgggccgcggggagcccccggagaaggagaaggggcggcggcgcctcaaggagagcttcgagaactaccgcaggaagcgggccctcaggaagatgcagcacggctcgaggcagggggaggtggactgggagaacaccacgggcagcgacaacaccgacaccgagggcccctagccggcccctccgtccagccgcctgggacctggccgctgccccgctaggccgcctttgttgacgagcacgggcgtgttctgtctctggggaatgcacacgcacgttgcagtgttaaaaacgcagccgcgtcaataaacgagcaccgtccgga

SEQ ID NO: 9 - DNA, 1814 bp, homo sapiens
gactgtggagttaagcagaacccatcaggaagtgcacaggcgtccggcgtgctcctccctccctgcagccccgggcagcatctcccagaggctccgcggcccaggctcctggtgtgtctgcagtgcaggtggctcctggaagaccctcagcctgcctgctgaggccatgtcggactacgagaacgatgacgagtgctggagcgtcctggagggcttccgggtgacgctcacctcggtcatcgacccctcacgcatcacaccttacctgcggcagtgcaaggtcctgaaccccgatgatgaggagcaggtgctcagcgaccccaacctggtcatccgcaaacggaaagtgggtgtgctcctggacatcctgcagcggaccggccacaagggctacgtggccttcctcgagagcctggagctctactacccgcagctgtacaagaaggtcacaggcaaggagccggcccgcgtcttctccatgatcatcgacgcgtccggggagtcaggcctgactcagctgctgatgactgaggtcatgaagctgcagaagaaggtgcaggacctgaccgcgctgctgagctccaaagatgacttcatcaaggagctgcgggtgaaggacagcctgctgcgcaagcaccaggagcgtgtgcagaggctcaaggaggagtgcgaggccggcagccgcgagctcaagcgctgcaaggaggagaactacgacctggccatgcgcctggcgcaccagagtgaggagaagggcgccgcgctcatgcggaaccgtgacctgcagctggagattgaccagctcaagcacagcctcatgaaggccgaggacgactgcaaggtggagcgcaagcacacgctgaagctcaggcacgccatggagcagcggcccagccaggagctgctgtgggagctgcagcaggagaaggccctgctccaggcccgggtgcaggagctggaggcctccgtccaggaggggaagctggacaggagcagcccctacatccaggtactggaggaggactggcggcaggcgctgcgggaccaccaggagcaggccaacaccatcttctccctgcgcaaggacctccgccagggcgaggcccgacgcctccggtgcatggaggagaaggagatgttcgagctgcagtgcctggcactacgtaaggactccaagatgtacaaggaccgcatcgaggccatcctgctgcagatggaggaggtcgccattgagcgggaccaggccatagccacgcgggaggagctgcacgcacagcacgcccggggcctgcaggagaaggacgcgctgcgcaagcaggtgcgggagctgggcgagaaggcggatgagctgcagctgcaggtgttccagtgtgaggcgcagctactggccgtggagggcaggctcaggcggcagcagctggagacgctcgtcctgagctccgacctggaagatggctcacccaggaggtcccaggagctctcactcccccaggacctggaggacacccagctctcagacaaaggctgccttgccggcggggggagcccgaaacagccctttgcagctctgcaccaggagcaggttttgcggaacccccatgacgcaggcccagccggactgccgggcattggggccgtttgttaagcggcactcattttgcggaggccatgcgggtgctcaccacccccatgcacacgccatctgtgtaacttcaggatctgttctgtttcaccatgtaacacacaatacatgcatgcattgtattagtgttagaaaacacagctgcgtaaataaacagcacgggtgacccgca

SEQ ID NO: 10 - DNA, 30 bp, homo sapiens
cccttgtctgtcaaaactgtcctgaatggg

SEQ ID NO: 11 - DNA, 18 bp, homo sapiens
gtcccaacatgggtagtt

SEQ ID NO: 12 - DNA, 18 bp, homo sapiens
ggccactcttgcatcatc

SEQ ID NO: 13 - DNA, 17 bp, homo sapiens
aacctgctctcacccag

SEQ ID NO: 14 - DNA, 25 bp, homo sapiens
gttcctctctcagaccccatctgtg

SEQ ID NO: 15 - DNA, 25 bp, homo sapiens
aggccatgtcggactacgagaacga

SEQ ID NO: 16 - DNA, 17 bp, homo sapiens
tcggtcatcgacccctc

SEQ ID NO: 17 - DNA, 24 bp, homo sapiens
aaggtcctgaaccccgatgatgag

SEQ ID NO: 18 - DNA, 29 bp, homo sapiens
ccaacctggtcatccgcaaacggaaagtg

SEQ ID NO: 19 - DNA, 24 bp, homo sapiens
gtccccagcctagtaccaagaccc

SEQ ID NO: 20 - DNA, 32 bp, homo sapiens
gtgctcctggacatcctgcagcggaccggcca

SEQ ID NO: 21 - DNA, 63 bp, homo sapiens
gggctacgtggccttcctcgagagcctggagctctactacccgcagctgtacaagaaggtcac

SEQ ID NO: 22 - DNA, 31 bp, homo sapiens
cgcgtcttctccatgatcatcggtgagtgac

SEQ ID NO: 23 - DNA, 17 bp, homo sapiens
ccatccctaggagccct

SEQ ID NO: 24 - DNA, 21 bp, homo sapiens
gatgacttcatcaaggagctg

SEQ ID NO: 25 - DNA, 26 bp, homo sapiens
gaggagaactacgacctggccatgcg

SEQ ID NO: 26 - DNA, 21 bp, homo sapiens
cgctcatgcggaaccgtgacc

SEQ ID NO: 27 - DNA, 17 bp, homo sapiens
ctcaagcacagcctcat

SEQ ID NO: 28 - DNA, 25 bp, homo sapiens
cattgccttttgcccccttcaggag

SEQ ID NO: 29 - DNA, 23 bp, homo sapiens
gagcagcccctacatccaggtac

SEQ ID NO: 30 - DNA, 42 bp, homo sapiens
caggccaacaccatcttctccctgcgcaaggacctccgccag

SEQ ID NO: 31 - DNA, 25 bp, homo sapiens
gcgaggcccgacgcctccgggtagg

SEQ ID NO: 32 - DNA, 16 bp, homo sapiens
cgcccactccgtgcct

SEQ ID NO: 33 - DNA, 17 bp, homo sapiens
aggagatgttcgagctg

SEQ ID NO: 34 - DNA, 41 bp, homo sapiens
aaggactccaagatgtacaaggaccgcatcgaggccatcct

SEQ ID NO: 35 - DNA, 20 bp, homo sapiens
atctttgttatttgtttttg

SEQ ID NO: 36 - DNA, 20 bp, homo sapiens
tgatgaagtcaatacttccc

SEQ ID NO: 37 - DNA, 24 bp, homo sapiens
agggaaaaccgtgttcaaccttcc

SEQ ID NO: 38 - DNA, 18 bp, homo sapiens
gctccaccttacagactt

SEQ ID NO: 39 - DNA, 41 bp, homo sapiens
aaacattctgtcttgttttaccagtagctttttttaatctt

SEQ ID NO: 40 - DNA, 17 bp, homo sapiens
caagtcaccattgcgct

SEQ ID NO: 41 - DNA, 35 bp, homo sapiens
tccacatcaggagccttaaaacgagacccctgggg

SEQ ID NO: 42 - DNA, 28 bp, homo sapiens
tccaaaatttatcaaatgtgcacgtgtg

SEQ ID NO: 43 - DNA, 16 bp, homo sapiens
cacgctgtgtccaccg

SEQ ID NO: 44 - DNA, 18 bp, homo sapiens
cggcttgacgtcctccgg

SEQ ID NO: 45 - DNA, 23 bp, homo sapiens
tggaggatcccgctctgtgccct

SEQ ID NO: 46 - DNA, 23 bp, homo sapiens
ttttctatgaccacagagctccg

SEQ ID NO: 47 - DNA, 21 bp, homo sapiens
ctggccttccttcacctgggg

SEQ ID NO: 48 - DNA, 18 bp, homo sapiens
cccagctctcagacaaag

SEQ ID NO: 49 - DNA, 34 bp, homo sapiens
gcccatcttcagcacaggcagcccgtgccgcaat

SEQ ID NO: 50 - DNA, 21 bp, homo sapiens
ggctggggataagtaaaatgg

SEQ ID NO: 51 - DNA, 20 bp, homo sapiens
gagaacaaactacagagccc

SEQ ID NO: 52 - DNA, 17 bp, homo sapiens
ctgtgtcccggtgcagt

SEQ ID NO: 53 - DNA, 16 bp, homo sapiens
ggggcttctagcgggc

SEQ ID NO: 54 - DNA, 23 bp, homo sapiens
gtggtgatgaggtaggtgtttgc

SEQ ID NO: 55 - DNA, 17 bp, homo sapiens
caagcccccatgtaggc

SEQ ID NO: 56 - DNA, 21 bp, homo sapiens
ctgaaggttctctcccaattg

SEQ ID NO: 57 - DNA, 28 bp, homo sapiens
catgcccacagatgctttggagtgatgg

SEQ ID NO: 58 - DNA, 23 bp, homo sapiens
cagagtctacactggacccatgt

SEQ ID NO: 59 - DNA, 23 bp, homo sapiens
caggcacgactctcctttccagg

SEQ ID NO: 60 - DNA, 29 bp, homo sapiens
cctcgggctttgttgtagaaacaatggcc

SEQ ID NO: 61 - DNA, 41 bp, homo sapiens
tgtgtcttggcatctgaaatgcaggctacccacaccggctc

SEQ ID NO: 62 - DNA, 20 bp, homo sapiens
agaactaccgcaggtaggcg

SEQ ID NO: 63 - DNA, 28 bp, homo sapiens
ccccaggcttctccaaaacgggctgggg

SEQ ID NO: 64 - DNA, 19 bp, homo sapiens
gcagcgacaacaccgacac

SEQ ID NO: 65 - DNA, 19 bp, homo sapiens
gaatctggtgccctgaaag

SEQ ID NO: 66 - DNA, 19 bp, homo sapiens
gtttgttaagcggcactca

SEQ ID NO: 67 - DNA, 23 bp, homo sapiens
catgcacacgccatctgtgtaac

SEQ ID NO: 68 - DNA, 29 bp, homo sapiens
tttcaccatgtaacacacaatacatgcat

SEQ ID NO: 69 - DNA, 19 bp, homo sapiens
taaataaacagcacgggtg

SEQ ID NO: 70 - DNA, 19 bp, Artificial Sequence
  Antisense oligonucleotide
aggacagttttgacagaca

SEQ ID NO: 71 - DNA, 19 bp, Artificial Sequence
  Antisense oligonucleotide
tcaggacagttttgacaga

SEQ ID NO: 72 - DNA, 20 bp, Artificial Sequence
  Antisense oligonucleotide
attcaggacagttttgacag

SEQ ID NO: 73 - DNA, 19 bp, Artificial Sequence
  Antisense oligonucleotide
attcaggacagttttgaca

SEQ ID NO: 74 - DNA, 18 bp, Artificial Sequence
  Antisense oligonucleotide
attcaggacagttttgac

SEQ ID NO: 75 - DNA, 19 bp, Artificial Sequence
  Antisense oligonucleotide
cattcaggacagttttgac

SEQ ID NO: 76 - DNA, 20 bp, Artificial Sequence
  Antisense oligonucleotide
ccattcaggacagttttgac

SEQ ID NO: 77 - DNA, 18 bp, Artificial Sequence
  Antisense oligonucleotide
cattcaggacagttttga

SEQ ID NO: 78 - DNA, 19 bp, Artificial Sequence
  Antisense oligonucleotide
ccattcaggacagttttga

SEQ ID NO: 79 - DNA, 18 bp, Artificial Sequence
  Antisense oligonucleotide
ccattcaggacagttttg

SEQ ID NO: 80 - DNA, 19 bp, Artificial Sequence
  Antisense oligonucleotide
cccattcaggacagttttg

SEQ ID NO: 81 - DNA, 18 bp, Artificial Sequence
  Antisense oligonucleotide
cccattcaggacagtttt

SEQ ID NO: 82 - DNA, 17 bp, Artificial Sequence
  Antisense oligonucleotide
cccattcaggacagttt

SEQ ID NO: 83 - DNA, 16 bp, Artificial Sequence
  Antisense oligonucleotide
ctacccatgttgggac

SEQ ID NO: 84 - DNA, 17 bp, Artificial Sequence
  Antisense oligonucleotide
actacccatgttgggac

SEQ ID NO: 85 - DNA, 18 bp, Artificial Sequence
  Antisense oligonucleotide
aactacccatgttgggac

SEQ ID NO: 86 - DNA, 17 bp, Artificial Sequence
  Antisense oligonucleotide
aactacccatgttggga

SEQ ID NO: 87 - DNA, 17 bp, Artificial Sequence
  Antisense oligonucleotide
atgatgcaagagtggcc

SEQ ID NO: 88 - DNA, 17 bp, Artificial Sequence
  Antisense oligonucleotide
ctgggtgagagcaggtt

SEQ ID NO: 89 - DNA, 18 bp, Artificial Sequence
  Antisense oligonucleotide
ggggtctgagagaggaac

SEQ ID NO: 90 - DNA, 18 bp, Artificial Sequence
  Antisense oligonucleotide
tggggtctgagagaggaa

SEQ ID NO: 91 - DNA, 19 bp, Artificial Sequence
  Antisense oligonucleotide
atggggtctgagagaggaa

SEQ ID NO: 92 - DNA, 20 bp, Artificial Sequence
  Antisense oligonucleotide
gatggggtctgagagaggaa

SEQ ID NO: 93 - DNA, 18 bp, Artificial Sequence
  Antisense oligonucleotide
atggggtctgagagagga

SEQ ID NO: 94 - DNA, 19 bp, Artificial Sequence
  Antisense oligonucleotide
agatggggtctgagagagg

SEQ ID NO: 95 - DNA, 17 bp, Artificial Sequence
  Antisense oligonucleotide
gatggggtctgagagag

SEQ ID NO: 96 - DNA, 18 bp, Artificial Sequence
  Antisense oligonucleotide
agatggggtctgagagag

SEQ ID NO: 97 - DNA, 19 bp, Artificial Sequence
  Antisense oligonucleotide
cagatggggtctgagagag

SEQ ID NO: 98 - DNA, 20 bp, Artificial Sequence
  Antisense oligonucleotide
acagatggggtctgagagag

SEQ ID NO: 99 - DNA, 18 bp, Artificial Sequence
  Antisense oligonucleotide
cagatggggtctgagaga

SEQ ID NO: 100 - DNA, 18 bp, Artificial Sequence
  Antisense oligonucleotide
acagatggggtctgagag

SEQ ID NO: 101 - DNA, 19 bp, Artificial Sequence
  Antisense oligonucleotide
cacagatggggtctgagag

SEQ ID NO: 102 - DNA, 16 bp, Artificial Sequence
  Antisense oligonucleotide
tagtccgacatggcct

SEQ ID NO: 103 - DNA, 15 bp, Artificial Sequence
  Antisense oligonucleotide
tagtccgacatggcc

SEQ ID NO: 104 - DNA, 18 bp, Artificial Sequence
  Antisense oligonucleotide
ttctcgtagtccgacatg

SEQ ID NO: 105 - DNA, 19 bp, Artificial Sequence
  Antisense oligonucleotide
gttctcgtagtccgacatg

SEQ ID NO: 106 - DNA, 16 bp, Artificial Sequence
  Antisense oligonucleotide
tctcgtagtccgacat

SEQ ID NO: 107 - DNA, 17 bp, Artificial Sequence
  Antisense oligonucleotide
ttctcgtagtccgacat

SEQ ID NO: 108 - DNA, 18 bp, Artificial Sequence
  Antisense oligonucleotide
gttctcgtagtccgacat

SEQ ID NO: 109 - DNA, 19 bp, Artificial Sequence
  Antisense oligonucleotide
cgttctcgtagtccgacat

SEQ ID NO: 110 - DNA, 17 bp, Artificial Sequence
  Antisense oligonucleotide
gttctcgtagtccgaca

SEQ ID NO: 111 - DNA, 18 bp, Artificial Sequence
  Antisense oligonucleotide
cgttctcgtagtccgaca

SEQ ID NO: 112 - DNA, 16 bp, Artificial Sequence
  Antisense oligonucleotide
cgttctcgtagtccga

SEQ ID NO: 113 - DNA, 15 bp, Artificial Sequence
  Antisense oligonucleotide
ggggtcgatgaccga

SEQ ID NO: 114 - DNA, 16 bp, Artificial Sequence
  Antisense oligonucleotide
aggggtcgatgaccga

SEQ ID NO: 115 - DNA, 15 bp, Artificial Sequence
  Antisense oligonucleotide
aggggtcgatgaccg

SEQ ID NO: 116 - DNA, 16 bp, Artificial Sequence
  Antisense oligonucleotide
gaggggtcgatgaccg

SEQ ID NO: 117 - DNA, 17 bp, Artificial Sequence
  Antisense oligonucleotide
tcggggttcaggacctt

SEQ ID NO: 118 - DNA, 16 bp, Artificial Sequence
  Antisense oligonucleotide
catcggggttcaggac

SEQ ID NO: 119 - DNA, 17 bp, Artificial Sequence
  Antisense oligonucleotide
tcatcggggttcaggac

SEQ ID NO: 120 - DNA, 18 bp, Artificial Sequence
  Antisense oligonucleotide
atcatcggggttcaggac

SEQ ID NO: 121 - DNA, 19 bp, Artificial Sequence
  Antisense oligonucleotide
catcatcggggttcaggac

SEQ ID NO: 122 - DNA, 17 bp, Artificial Sequence
  Antisense oligonucleotide
atcatcggggttcagga

SEQ ID NO: 123 - DNA, 18 bp, Artificial Sequence
  Antisense oligonucleotide
catcatcggggttcagga

SEQ ID NO: 124 - DNA, 16 bp, Artificial Sequence
  Antisense oligonucleotide
atcatcggggttcagg

SEQ ID NO: 125 - DNA, 17 bp, Artificial Sequence
  Antisense oligonucleotide
catcatcggggttcagg

SEQ ID NO: 126 - DNA, 16 bp, Artificial Sequence
  Antisense oligonucleotide
catcatcggggttcag

SEQ ID NO: 127 - DNA, 17 bp, Artificial Sequence
  Antisense oligonucleotide
tcatcatcggggttcag

SEQ ID NO: 128 - DNA, 18 bp, Artificial Sequence
  Antisense oligonucleotide
ctcatcatcggggttcag

SEQ ID NO: 129 - DNA, 16 bp, Artificial Sequence
  Antisense oligonucleotide
tcatcatcggggttca

SEQ ID NO: 130 - DNA, 16 bp, Artificial Sequence
  Antisense oligonucleotide
cggatgaccaggttgg

SEQ ID NO: 131 - DNA, 16 bp, Artificial Sequence
  Antisense oligonucleotide
gcggatgaccaggttg

SEQ ID NO: 132 - DNA, 17 bp, Artificial Sequence
  Antisense oligonucleotide
tgcggatgaccaggttg

SEQ ID NO: 133 - DNA, 18 bp, Artificial Sequence
  Antisense oligonucleotide
ttgcggatgaccaggttg

SEQ ID NO: 134 - DNA, 16 bp, Artificial Sequence
  Antisense oligonucleotide
tgcggatgaccaggtt

SEQ ID NO: 135 - DNA, 17 bp, Artificial Sequence
  Antisense oligonucleotide
ttgcggatgaccaggtt

SEQ ID NO: 136 - DNA, 16 bp, Artificial Sequence
  Antisense oligonucleotide
ttgcggatgaccaggt

SEQ ID NO: 137 - DNA, 16 bp, Artificial Sequence
  Antisense oligonucleotide
cgtttgcggatgacca

SEQ ID NO: 138 - DNA, 17 bp, Artificial Sequence
  Antisense oligonucleotide
ccgtttgcggatgacca

SEQ ID NO: 139 - DNA, 15 bp, Artificial Sequence
  Antisense oligonucleotide
cgtttgcggatgacc

SEQ ID NO: 140 - DNA, 16 bp, Artificial Sequence
  Antisense oligonucleotide
ccgtttgcggatgacc

SEQ ID NO: 141 - DNA, 16 bp, Artificial Sequence
  Antisense oligonucleotide
ttccgtttgcggatga

SEQ ID NO: 142 - DNA, 17 bp, Artificial Sequence
  Antisense oligonucleotide
tttccgtttgcggatga

SEQ ID NO: 143 - DNA, 18 bp, Artificial Sequence
  Antisense oligonucleotide
ctttccgtttgcggatga

SEQ ID NO: 144 - DNA, 15 bp, Artificial Sequence
  Antisense oligonucleotide
ttccgtttgcggatg

SEQ ID NO: 145 - DNA, 17 bp, Artificial Sequence
  Antisense oligonucleotide
ctttccgtttgcggatg

SEQ ID NO: 146 - DNA, 18 bp, Artificial Sequence
  Antisense oligonucleotide
actttccgtttgcggatg

SEQ ID NO: 147 - DNA, 16 bp, Artificial Sequence
  Antisense oligonucleotide
ctttccgtttgcggat

SEQ ID NO: 148 - DNA, 17 bp, Artificial Sequence
  Antisense oligonucleotide
actttccgtttgcggat

SEQ ID NO: 149 - DNA, 17 bp, Artificial Sequence
  Antisense oligonucleotide
ggtactaggctggggac

SEQ ID NO: 150 - DNA, 17 bp, Artificial Sequence
  Antisense oligonucleotide
tggtactaggctgggga

SEQ ID NO: 151 - DNA, 18 bp, Artificial Sequence
  Antisense oligonucleotide
ttggtactaggctgggga

SEQ ID NO: 152 - DNA, 17 bp, Artificial Sequence
  Antisense oligonucleotide
ttggtactaggctgggg

SEQ ID NO: 153 - DNA, 18 bp, Artificial Sequence
  Antisense oligonucleotide
tcttggtactaggctggg

SEQ ID NO: 154 - DNA, 17 bp, Artificial Sequence
  Antisense oligonucleotide
gtcttggtactaggctg

SEQ ID NO: 155 - DNA, 18 bp, Artificial Sequence
  Antisense oligonucleotide
ggtcttggtactaggctg

SEQ ID NO: 156 - DNA, 16 bp, Artificial Sequence
  Antisense oligonucleotide
gtcttggtactaggct

SEQ ID NO: 157 - DNA, 17 bp, Artificial Sequence
  Antisense oligonucleotide
ggtcttggtactaggct

SEQ ID NO: 158 - DNA, 16 bp, Artificial Sequence
  Antisense oligonucleotide
ggtcttggtactaggc

SEQ ID NO: 159 - DNA, 18 bp, Artificial Sequence
  Antisense oligonucleotide
caggatgtccaggagcac

SEQ ID NO: 160 - DNA, 20 bp, Artificial Sequence
  Antisense oligonucleotide
cgaggaaggccacgtagccc

SEQ ID NO: 161 - DNA, 17 bp, Artificial Sequence
  Antisense oligonucleotide
cgaggaaggccacgtag

SEQ ID NO: 162 - DNA, 17 bp, Artificial Sequence
  Antisense oligonucleotide
ctcgaggaaggccacgt

SEQ ID NO: 163 - DNA, 16 bp, Artificial Sequence
  Antisense oligonucleotide
ctcgaggaaggccacg

SEQ ID NO: 164 - DNA, 17 bp, Artificial Sequence
  Antisense oligonucleotide
ctctcgaggaaggccac

SEQ ID NO: 165 - DNA, 17 bp, Artificial Sequence
  Antisense oligonucleotide
agtagagctccaggctc

SEQ ID NO: 166 - DNA, 18 bp, Artificial Sequence
  Antisense oligonucleotide
tagtagagctccaggctc

SEQ ID NO: 167 - DNA, 17 bp, Artificial Sequence
  Antisense oligonucleotide
tagtagagctccaggct

SEQ ID NO: 168 - DNA, 18 bp, Artificial Sequence
  Antisense oligonucleotide
cgggtagtagagctccag

SEQ ID NO: 169 - DNA, 17 bp, Artificial Sequence
  Antisense oligonucleotide
cgggtagtagagctcca

SEQ ID NO: 170 - DNA, 16 bp, Artificial Sequence
  Antisense oligonucleotide
cgggtagtagagctcc

SEQ ID NO: 171 - DNA, 16 bp, Artificial Sequence
  Antisense oligonucleotide
gcgggtagtagagctc

SEQ ID NO: 172 - DNA, 17 bp, Artificial Sequence
  Antisense oligonucleotide
tgcgggtagtagagctc

SEQ ID NO: 173 - DNA, 18 bp, Artificial Sequence
  Antisense oligonucleotide
ctgcgggtagtagagctc

SEQ ID NO: 174 - DNA, 19 bp, Artificial Sequence
  Antisense oligonucleotide
gctgcgggtagtagagctc

SEQ ID NO: 175 - DNA, 16 bp, Artificial Sequence
  Antisense oligonucleotide
tgcgggtagtagagct

SEQ ID NO: 176 - DNA, 17 bp, Artificial Sequence
  Antisense oligonucleotide
ctgcgggtagtagagct

SEQ ID NO: 177 - DNA, 16 bp, Artificial Sequence
  Antisense oligonucleotide
ctgcgggtagtagagc

SEQ ID NO: 178 - DNA, 17 bp, Artificial Sequence
  Antisense oligonucleotide
gctgcgggtagtagagc

SEQ ID NO: 179 - DNA, 18 bp, Artificial Sequence
  Antisense oligonucleotide
agctgcgggtagtagagc

SEQ ID NO: 180 - DNA, 16 bp, Artificial Sequence
  Antisense oligonucleotide
gctgcgggtagtagag

SEQ ID NO: 181 - DNA, 17 bp, Artificial Sequence
  Antisense oligonucleotide
agctgcgggtagtagag

SEQ ID NO: 182 - DNA, 15 bp, Artificial Sequence
  Antisense oligonucleotide
gctgcgggtagtaga

SEQ ID NO: 183 - DNA, 16 bp, Artificial Sequence
  Antisense oligonucleotide
agctgcgggtagtaga

SEQ ID NO: 184 - DNA, 19 bp, Artificial Sequence
  Antisense oligonucleotide
tgaccttcttgtacagctg

SEQ ID NO: 185 - DNA, 17 bp, Artificial Sequence
  Antisense oligonucleotide
gaccttcttgtacagct

SEQ ID NO: 186 - DNA, 18 bp, Artificial Sequence
  Antisense oligonucleotide
tgaccttcttgtacagct

SEQ ID NO: 187 - DNA, 17 bp, Artificial Sequence
  Antisense oligonucleotide
tgaccttcttgtacagc

SEQ ID NO: 188 - DNA, 16 bp, Artificial Sequence
  Antisense oligonucleotide
tcatggagaagacgcg

SEQ ID NO: 189 - DNA, 17 bp, Artificial Sequence
  Antisense oligonucleotide
atcatggagaagacgcg

SEQ ID NO: 190 - DNA, 18 bp, Artificial Sequence
  Antisense oligonucleotide
gatcatggagaagacgcg

SEQ ID NO: 191 - DNA, 19 bp, Artificial Sequence
  Antisense oligonucleotide
tgatcatggagaagacgcg

SEQ ID NO: 192 - DNA, 20 bp, Artificial Sequence
  Antisense oligonucleotide
atgatcatggagaagacgcg

SEQ ID NO: 193 - DNA, 17 bp, Artificial Sequence
  Antisense oligonucleotide
gatcatggagaagacgc

SEQ ID NO: 194 - DNA, 18 bp, Artificial Sequence
  Antisense oligonucleotide
tgatcatggagaagacgc

SEQ ID NO: 195 - DNA, 19 bp, Artificial Sequence
  Antisense oligonucleotide
atgatcatggagaagacgc

SEQ ID NO: 196 - DNA, 17 bp, Artificial Sequence
  Antisense oligonucleotide
tgatcatggagaagacg

SEQ ID NO: 197 - DNA, 18 bp, Artificial Sequence
  Antisense oligonucleotide
atgatcatggagaagacg

SEQ ID NO: 198 - DNA, 20 bp, Artificial Sequence
  Antisense oligonucleotide
ccgatgatcatggagaagac

SEQ ID NO: 199 - DNA, 18 bp, Artificial Sequence
  Antisense oligonucleotide
cgatgatcatggagaaga

SEQ ID NO: 200 - DNA, 19 bp, Artificial Sequence
  Antisense oligonucleotide
ccgatgatcatggagaaga

SEQ ID NO: 201 - DNA, 19 bp, Artificial Sequence
  Antisense oligonucleotide
accgatgatcatggagaag

SEQ ID NO: 202 - DNA, 20 bp, Artificial Sequence
  Antisense oligonucleotide
caccgatgatcatggagaag

SEQ ID NO: 203 - DNA, 18 bp, Artificial Sequence
  Antisense oligonucleotide
accgatgatcatggagaa

SEQ ID NO: 204 - DNA, 19 bp, Artificial Sequence
  Antisense oligonucleotide
caccgatgatcatggagaa

SEQ ID NO: 205 - DNA, 16 bp, Artificial Sequence
  Antisense oligonucleotide
ccgatgatcatggaga

SEQ ID NO: 206 - DNA, 17 bp, Artificial Sequence
  Antisense oligonucleotide
accgatgatcatggaga

SEQ ID NO: 207 - DNA, 16 bp, Artificial Sequence
  Antisense oligonucleotide
accgatgatcatggag

SEQ ID NO: 208 - DNA, 17 bp, Artificial Sequence
  Antisense oligonucleotide
caccgatgatcatggag

SEQ ID NO: 209 - DNA, 18 bp, Artificial Sequence
  Antisense oligonucleotide
tcaccgatgatcatggag

SEQ ID NO: 210 - DNA, 16 bp, Artificial Sequence
  Antisense oligonucleotide
caccgatgatcatgga

SEQ ID NO: 211 - DNA, 16 bp, Artificial Sequence
  Antisense oligonucleotide
tcaccgatgatcatgg

SEQ ID NO: 212 - DNA, 17 bp, Artificial Sequence
  Antisense oligonucleotide
ctcaccgatgatcatgg

SEQ ID NO: 213 - DNA, 17 bp, Artificial Sequence
  Antisense oligonucleotide
actcaccgatgatcatg

SEQ ID NO: 214 - DNA, 18 bp, Artificial Sequence
  Antisense oligonucleotide
cactcaccgatgatcatg

SEQ ID NO: 215 - DNA, 16 bp, Artificial Sequence
  Antisense oligonucleotide
actcaccgatgatcat

SEQ ID NO: 216 - DNA, 17 bp, Artificial Sequence
  Antisense oligonucleotide
cactcaccgatgatcat

SEQ ID NO: 217 - DNA, 18 bp, Artificial Sequence
  Antisense oligonucleotide
tcactcaccgatgatcat

SEQ ID NO: 218 - DNA, 17 bp, Artificial Sequence
  Antisense oligonucleotide
tcactcaccgatgatca

SEQ ID NO: 219 - DNA, 18 bp, Artificial Sequence
  Antisense oligonucleotide
gtcactcaccgatgatca

SEQ ID NO: 220 - DNA, 16 bp, Artificial Sequence
  Antisense oligonucleotide
tcactcaccgatgatc

SEQ ID NO: 221 - DNA, 17 bp, Artificial Sequence
  Antisense oligonucleotide
gtcactcaccgatgatc

SEQ ID NO: 222 - DNA, 17 bp, Artificial Sequence
  Antisense oligonucleotide
agggctcctagggatgg

SEQ ID NO: 223 - DNA, 20 bp, Artificial Sequence
  Antisense oligonucleotide
agctccttgatgaagtcatc

SEQ ID NO: 224 - DNA, 19 bp, Artificial Sequence
  Antisense oligonucleotide
agctccttgatgaagtcat

SEQ ID NO: 225 - DNA, 20 bp, Artificial Sequence
  Antisense oligonucleotide
cagctccttgatgaagtcat

SEQ ID NO: 226 - DNA, 17 bp, Artificial Sequence
  Antisense oligonucleotide
aggtcgtagttctcctc

SEQ ID NO: 227 - DNA, 16 bp, Artificial Sequence
  Antisense oligonucleotide
ggccaggtcgtagttc

SEQ ID NO: 228 - DNA, 17 bp, Artificial Sequence
  Antisense oligonucleotide
tggccaggtcgtagttc

SEQ ID NO: 229 - DNA, 18 bp, Artificial Sequence
  Antisense oligonucleotide
atggccaggtcgtagttc

SEQ ID NO: 230 - DNA, 16 bp, Artificial Sequence
  Antisense oligonucleotide
tggccaggtcgtagtt

SEQ ID NO: 231 - DNA, 17 bp, Artificial Sequence
  Antisense oligonucleotide
atggccaggtcgtagtt

SEQ ID NO: 232 - DNA, 18 bp, Artificial Sequence
  Antisense oligonucleotide
catggccaggtcgtagtt

SEQ ID NO: 233 - DNA, 16 bp, Artificial Sequence
  Antisense oligonucleotide
catggccaggtcgtag

SEQ ID NO: 234 - DNA, 14 bp, Artificial Sequence
  Antisense oligonucleotide
gttccgcatgagcg

SEQ ID NO: 235 - DNA, 16 bp, Artificial Sequence
  Antisense oligonucleotide
cggttccgcatgagcg

SEQ ID NO: 236 - DNA, 15 bp, Artificial Sequence
  Antisense oligonucleotide
cggttccgcatgagc

SEQ ID NO: 237 - DNA, 15 bp, Artificial Sequence
  Antisense oligonucleotide
acggttccgcatgag

SEQ ID NO: 238 - DNA, 16 bp, Artificial Sequence
  Antisense oligonucleotide
cacggttccgcatgag

SEQ ID NO: 239 - DNA, 15 bp, Artificial Sequence
  Antisense oligonucleotide
gtcacggttccgcat

SEQ ID NO: 240 - DNA, 16 bp, Artificial Sequence
  Antisense oligonucleotide
ggtcacggttccgcat

SEQ ID NO: 241 - DNA, 19 bp, Artificial Sequence
  Antisense oligonucleotide
aagggggcaaaaggcaatg

SEQ ID NO: 242 - DNA, 18 bp, Artificial Sequence
  Antisense oligonucleotide
aagggggcaaaaggcaat

SEQ ID NO: 243 - DNA, 19 bp, Artificial Sequence
  Antisense oligonucleotide
gaagggggcaaaaggcaat

SEQ ID NO: 244 - DNA, 20 bp, Artificial Sequence
  Antisense oligonucleotide
tgaagggggcaaaaggcaat

SEQ ID NO: 245 - DNA, 18 bp, Artificial Sequence
  Antisense oligonucleotide
gaagggggcaaaaggcaa

SEQ ID NO: 246 - DNA, 19 bp, Artificial Sequence
  Antisense oligonucleotide
tgaagggggcaaaaggcaa

SEQ ID NO: 247 - DNA, 20 bp, Artificial Sequence
  Antisense oligonucleotide
ctgaagggggcaaaaggcaa

SEQ ID NO: 248 - DNA, 18 bp, Artificial Sequence
  Antisense oligonucleotide
tgaagggggcaaaaggca

SEQ ID NO: 249 - DNA, 19 bp, Artificial Sequence
  Antisense oligonucleotide
ctgaagggggcaaaaggca

SEQ ID NO: 250 - DNA, 17 bp, Artificial Sequence
  Antisense oligonucleotide
tgaagggggcaaaaggc

SEQ ID NO: 251 - DNA, 18 bp, Artificial Sequence
  Antisense oligonucleotide
ctgaagggggcaaaaggc

SEQ ID NO: 252 - DNA, 18 bp, Artificial Sequence
  Antisense oligonucleotide
tcctgaagggggcaaaag

SEQ ID NO: 253 - DNA, 19 bp, Artificial Sequence
  Antisense oligonucleotide
ctcctgaagggggcaaaag

SEQ ID NO: 254 - DNA, 18 bp, Artificial Sequence
  Antisense oligonucleotide
ctcctgaagggggcaaaa

SEQ ID NO: 255 - DNA, 17 bp, Artificial Sequence
  Antisense oligonucleotide
ctcctgaagggggcaaa

SEQ ID NO: 256 - DNA, 17 bp, Artificial Sequence
  Antisense oligonucleotide
ggatgtaggggctgctc

SEQ ID NO: 257 - DNA, 17 bp, Artificial Sequence
  Antisense oligonucleotide
ctggatgtaggggctgc

SEQ ID NO: 258 - DNA, 18 bp, Artificial Sequence
  Antisense oligonucleotide
gtacctggatgtaggggc

SEQ ID NO: 259 - DNA, 17 bp, Artificial Sequence
  Antisense oligonucleotide
aagatggtgttggcctg

SEQ ID NO: 260 - DNA, 20 bp, Artificial Sequence
  Antisense oligonucleotide
gggagaagatggtgttggcc

SEQ ID NO: 261 - DNA, 18 bp, Artificial Sequence
  Antisense oligonucleotide
ggagaagatggtgttggc

SEQ ID NO: 262 - DNA, 18 bp, Artificial Sequence
  Antisense oligonucleotide
gcgcagggagaagatggt

SEQ ID NO: 263 - DNA, 17 bp, Artificial Sequence
  Antisense oligonucleotide
tgcgcagggagaagatg

SEQ ID NO: 264 - DNA, 18 bp, Artificial Sequence
  Antisense oligonucleotide
ttgcgcagggagaagatg

SEQ ID NO: 265 - DNA, 18 bp, Artificial Sequence
  Antisense oligonucleotide
cttgcgcagggagaagat

SEQ ID NO: 266 - DNA, 17 bp, Artificial Sequence
  Antisense oligonucleotide
ccttgcgcagggagaag

SEQ ID NO: 267 - DNA, 16 bp, Artificial Sequence
  Antisense oligonucleotide
ccttgcgcagggagaa

SEQ ID NO: 268 - DNA, 17 bp, Artificial Sequence
  Antisense oligonucleotide
tccttgcgcagggagaa

SEQ ID NO: 269 - DNA, 16 bp, Artificial Sequence
  Antisense oligonucleotide
tggcggaggtccttgc

SEQ ID NO: 270 - DNA, 16 bp, Artificial Sequence
  Antisense oligonucleotide
ctggcggaggtccttg

SEQ ID NO: 271 - DNA, 18 bp, Artificial Sequence
  Antisense oligonucleotide
ggaggcgtcgggcctcgc

SEQ ID NO: 272 - DNA, 19 bp, Artificial Sequence
  Antisense oligonucleotide
cggaggcgtcgggcctcgc

SEQ ID NO: 273 - DNA, 18 bp, Artificial Sequence
  Antisense oligonucleotide
cggaggcgtcgggcctcg

SEQ ID NO: 274 - DNA, 17 bp, Artificial Sequence
  Antisense oligonucleotide
ccggaggcgtcgggcct

SEQ ID NO: 275 - DNA, 18 bp, Artificial Sequence
  Antisense oligonucleotide
acccggaggcgtcgggcc

SEQ ID NO: 276 - DNA, 19 bp, Artificial Sequence
  Antisense oligonucleotide
ctacccggaggcgtcgggc

SEQ ID NO: 277 - DNA, 20 bp, Artificial Sequence
  Antisense oligonucleotide
cctacccggaggcgtcgggc

SEQ ID NO: 278 - DNA, 18 bp, Artificial Sequence
  Antisense oligonucleotide
ctacccggaggcgtcggg

SEQ ID NO: 279 - DNA, 17 bp, Artificial Sequence
  Antisense oligonucleotide
ctacccggaggcgtcgg

SEQ ID NO: 280 - DNA, 15 bp, Artificial Sequence
  Antisense oligonucleotide
tacccggaggcgtcg

SEQ ID NO: 281 - DNA, 16 bp, Artificial Sequence
  Antisense oligonucleotide
ctacccggaggcgtcg

SEQ ID NO: 282 - DNA, 17 bp, Artificial Sequence
  Antisense oligonucleotide
cctacccggaggcgtcg

SEQ ID NO: 283 - DNA, 16 bp, Artificial Sequence
  Antisense oligonucleotide
aggcacggagtgggcg

SEQ ID NO: 284 - DNA, 17 bp, Artificial Sequence
  Antisense oligonucleotide
cagctcgaacatctcct

SEQ ID NO: 285 - DNA, 19 bp, Artificial Sequence
  Antisense oligonucleotide
tgtacatcttggagtcctt

SEQ ID NO: 286 - DNA, 20 bp, Artificial Sequence
  Antisense oligonucleotide
ttgtacatcttggagtcctt

SEQ ID NO: 287 - DNA, 18 bp, Artificial Sequence
  Antisense oligonucleotide
tgtacatcttggagtcct

SEQ ID NO: 288 - DNA, 19 bp, Artificial Sequence
  Antisense oligonucleotide
ttgtacatcttggagtcct

SEQ ID NO: 289 - DNA, 17 bp, Artificial Sequence
  Antisense oligonucleotide
tgtacatcttggagtcc

SEQ ID NO: 290 - DNA, 18 bp, Artificial Sequence
  Antisense oligonucleotide
ttgtacatcttggagtcc

SEQ ID NO: 291 - DNA, 19 bp, Artificial Sequence
  Antisense oligonucleotide
cttgtacatcttggagtcc

SEQ ID NO: 292 - DNA, 17 bp, Artificial Sequence
  Antisense oligonucleotide
ttgtacatcttggagtc

SEQ ID NO: 293 - DNA, 18 bp, Artificial Sequence
  Antisense oligonucleotide
cttgtacatcttggagtc

SEQ ID NO: 294 - DNA, 18 bp, Artificial Sequence
  Antisense oligonucleotide
ccttgtacatcttggagt

SEQ ID NO: 295 - DNA, 18 bp, Artificial Sequence
  Antisense oligonucleotide
tccttgtacatcttggag

SEQ ID NO: 296 - DNA, 17 bp, Artificial Sequence
  Antisense oligonucleotide
tccttgtacatcttgga

SEQ ID NO: 297 - DNA, 18 bp, Artificial Sequence
  Antisense oligonucleotide
gtccttgtacatcttgga

SEQ ID NO: 298 - DNA, 17 bp, Artificial Sequence
  Antisense oligonucleotide
tgcggtccttgtacatc

SEQ ID NO: 299 - DNA, 18 bp, Artificial Sequence
  Antisense oligonucleotide
atgcggtccttgtacatc

SEQ ID NO: 300 - DNA, 19 bp, Artificial Sequence
  Antisense oligonucleotide
gatgcggtccttgtacatc

SEQ ID NO: 301 - DNA, 16 bp, Artificial Sequence
  Antisense oligonucleotide
tgcggtccttgtacat

SEQ ID NO: 302 - DNA, 18 bp, Artificial Sequence
  Antisense oligonucleotide
gatgcggtccttgtacat

SEQ ID NO: 303 - DNA, 19 bp, Artificial Sequence
  Antisense oligonucleotide
cgatgcggtccttgtacat

SEQ ID NO: 304 - DNA, 17 bp, Artificial Sequence
  Antisense oligonucleotide
gatgcggtccttgtaca

SEQ ID NO: 305 - DNA, 18 bp, Artificial Sequence
  Antisense oligonucleotide
cgatgcggtccttgtaca

SEQ ID NO: 306 - DNA, 17 bp, Artificial Sequence
  Antisense oligonucleotide
aggatggcctcgatgcg

SEQ ID NO: 307 - DNA, 20 bp, Artificial Sequence
  Antisense oligonucleotide
caaaaacaaataacaaagat

SEQ ID NO: 308 - DNA, 17 bp, Artificial Sequence
  Antisense oligonucleotide
gaagtattgacttcatc

SEQ ID NO: 309 - DNA, 17 bp, Artificial Sequence
  Antisense oligonucleotide
ggaagtattgacttcat

SEQ ID NO: 310 - DNA, 18 bp, Artificial Sequence
  Antisense oligonucleotide
gggaagtattgacttcat

SEQ ID NO: 311 - DNA, 18 bp, Artificial Sequence
  Antisense oligonucleotide
ttgaacacggttttccct

SEQ ID NO: 312 - DNA, 17 bp, Artificial Sequence
  Antisense oligonucleotide
ttgaacacggttttccc

SEQ ID NO: 313 - DNA, 18 bp, Artificial Sequence
  Antisense oligonucleotide
gttgaacacggttttccc

SEQ ID NO: 314 - DNA, 19 bp, Artificial Sequence
  Antisense oligonucleotide
aggttgaacacggttttcc

SEQ ID NO: 315 - DNA, 20 bp, Artificial Sequence
  Antisense oligonucleotide
gaaggttgaacacggttttc

SEQ ID NO: 316 - DNA, 18 bp, Artificial Sequence
  Antisense oligonucleotide
aagtctgtaaggtggagc

SEQ ID NO: 317 - DNA, 19 bp, Artificial Sequence
  Antisense oligonucleotide
aaaacaagacagaatgttt

SEQ ID NO: 318 - DNA, 20 bp, Artificial Sequence
  Antisense oligonucleotide
taaaacaagacagaatgttt

SEQ ID NO: 319 - DNA, 20 bp, Artificial Sequence
  Antisense oligonucleotide
gtaaaacaagacagaatgtt

SEQ ID NO: 320 - DNA, 20 bp, Artificial Sequence
  Antisense oligonucleotide
ggtaaaacaagacagaatgt

SEQ ID NO: 321 - DNA, 20 bp, Artificial Sequence
  Antisense oligonucleotide
tggtaaaacaagacagaatg

SEQ ID NO: 322 - DNA, 19 bp, Artificial Sequence
  Antisense oligonucleotide
tggtaaaacaagacagaat

SEQ ID NO: 323 - DNA, 20 bp, Artificial Sequence
  Antisense oligonucleotide
ctggtaaaacaagacagaat

SEQ ID NO: 324 - DNA, 19 bp, Artificial Sequence
  Antisense oligonucleotide
ctggtaaaacaagacagaa

SEQ ID NO: 325 - DNA, 20 bp, Artificial Sequence
  Antisense oligonucleotide
actggtaaaacaagacagaa

SEQ ID NO: 326 - DNA, 18 bp, Artificial Sequence
  Antisense oligonucleotide
ctggtaaaacaagacaga

SEQ ID NO: 327 - DNA, 19 bp, Artificial Sequence
  Antisense oligonucleotide
actggtaaaacaagacaga

SEQ ID NO: 328 - DNA, 20 bp, Artificial Sequence
  Antisense oligonucleotide
tactggtaaaacaagacaga

SEQ ID NO: 329 - DNA, 18 bp, Artificial Sequence
  Antisense oligonucleotide
actggtaaaacaagacag

SEQ ID NO: 330 - DNA, 19 bp, Artificial Sequence
  Antisense oligonucleotide
tactggtaaaacaagacag

SEQ ID NO: 331 - DNA, 20 bp, Artificial Sequence
  Antisense oligonucleotide
ctactggtaaaacaagacag

SEQ ID NO: 332 - DNA, 18 bp, Artificial Sequence
  Antisense oligonucleotide
tactggtaaaacaagaca

SEQ ID NO: 333 - DNA, 19 bp, Artificial Sequence
  Antisense oligonucleotide
ctactggtaaaacaagaca

SEQ ID NO: 334 - DNA, 20 bp, Artificial Sequence
  Antisense oligonucleotide
gctactggtaaaacaagaca

SEQ ID NO: 335 - DNA, 17 bp, Artificial Sequence
  Antisense oligonucleotide
tactggtaaaacaagac

SEQ ID NO: 336 - DNA, 18 bp, Artificial Sequence
  Antisense oligonucleotide
ctactggtaaaacaagac

SEQ ID NO: 337 - DNA, 19 bp, Artificial Sequence
  Antisense oligonucleotide
gctactggtaaaacaagac

SEQ ID NO: 338 - DNA, 20 bp, Artificial Sequence
  Antisense oligonucleotide
agctactggtaaaacaagac

SEQ ID NO: 339 - DNA, 18 bp, Artificial Sequence
  Antisense oligonucleotide
gctactggtaaaacaaga

SEQ ID NO: 340 - DNA, 19 bp, Artificial Sequence
  Antisense oligonucleotide
agctactggtaaaacaaga

SEQ ID NO: 341 - DNA, 20 bp, Artificial Sequence
  Antisense oligonucleotide
aagctactggtaaaacaaga

SEQ ID NO: 342 - DNA, 17 bp, Artificial Sequence
  Antisense oligonucleotide
gctactggtaaaacaag

SEQ ID NO: 343 - DNA, 18 bp, Artificial Sequence
  Antisense oligonucleotide
agctactggtaaaacaag

SEQ ID NO: 344 - DNA, 19 bp, Artificial Sequence
  Antisense oligonucleotide
aagctactggtaaaacaag

SEQ ID NO: 345 - DNA, 20 bp, Artificial Sequence
  Antisense oligonucleotide
aaagctactggtaaaacaag

SEQ ID NO: 346 - DNA, 18 bp, Artificial Sequence
  Antisense oligonucleotide
aagctactggtaaaacaa

SEQ ID NO: 347 - DNA, 19 bp, Artificial Sequence
  Antisense oligonucleotide
aaagctactggtaaaacaa

SEQ ID NO: 348 - DNA, 20 bp, Artificial Sequence
  Antisense oligonucleotide
aaaagctactggtaaaacaa

SEQ ID NO: 349 - DNA, 18 bp, Artificial Sequence
  Antisense oligonucleotide
aaagctactggtaaaaca

SEQ ID NO: 350 - DNA, 19 bp, Artificial Sequence
  Antisense oligonucleotide
aaaagctactggtaaaaca

SEQ ID NO: 351 - DNA, 20 bp, Artificial Sequence
  Antisense oligonucleotide
aaaaagctactggtaaaaca

SEQ ID NO: 352 - DNA, 18 bp, Artificial Sequence
  Antisense oligonucleotide
aaaaaaagctactggtaa

SEQ ID NO: 353 - DNA, 18 bp, Artificial Sequence
  Antisense oligonucleotide
ttaaaaaaagctactggt

SEQ ID NO: 354 - DNA, 19 bp, Artificial Sequence
  Antisense oligonucleotide
attaaaaaaagctactggt

SEQ ID NO: 355 - DNA, 20 bp, Artificial Sequence
  Antisense oligonucleotide
gattaaaaaaagctactggt

SEQ ID NO: 356 - DNA, 18 bp, Artificial Sequence
  Antisense oligonucleotide
attaaaaaaagctactgg

SEQ ID NO: 357 - DNA, 19 bp, Artificial Sequence
  Antisense oligonucleotide
gattaaaaaaagctactgg

SEQ ID NO: 358 - DNA, 20 bp, Artificial Sequence
  Antisense oligonucleotide
agattaaaaaaagctactgg

SEQ ID NO: 359 - DNA, 18 bp, Artificial Sequence
  Antisense oligonucleotide
gattaaaaaaagctactg

SEQ ID NO: 360 - DNA, 19 bp, Artificial Sequence
  Antisense oligonucleotide
agattaaaaaaagctactg

SEQ ID NO: 361 - DNA, 20 bp, Artificial Sequence
  Antisense oligonucleotide
aagattaaaaaaagctactg

SEQ ID NO: 362 - DNA, 19 bp, Artificial Sequence
  Antisense oligonucleotide
aagattaaaaaaagctact

SEQ ID NO: 363 - DNA, 16 bp, Artificial Sequence
  Antisense oligonucleotide
agcgcaatggtgactt

SEQ ID NO: 364 - DNA, 19 bp, Artificial Sequence
  Antisense oligonucleotide
ttaaggctcctgatgtgga

SEQ ID NO: 365 - DNA, 20 bp, Artificial Sequence
  Antisense oligonucleotide
tttaaggctcctgatgtgga

SEQ ID NO: 366 - DNA, 18 bp, Artificial Sequence
  Antisense oligonucleotide
ttaaggctcctgatgtgg

SEQ ID NO: 367 - DNA, 19 bp, Artificial Sequence
  Antisense oligonucleotide
tttaaggctcctgatgtgg

SEQ ID NO: 368 - DNA, 17 bp, Artificial Sequence
  Antisense oligonucleotide
ttaaggctcctgatgtg

SEQ ID NO: 369 - DNA, 18 bp, Artificial Sequence
  Antisense oligonucleotide
tttaaggctcctgatgtg

SEQ ID NO: 370 - DNA, 19 bp, Artificial Sequence
  Antisense oligonucleotide
ttttaaggctcctgatgtg

SEQ ID NO: 371 - DNA, 19 bp, Artificial Sequence
  Antisense oligonucleotide
tctcgttttaaggctcctg

SEQ ID NO: 372 - DNA, 17 bp, Artificial Sequence
  Antisense oligonucleotide
ggtctcgttttaaggct

SEQ ID NO: 373 - DNA, 19 bp, Artificial Sequence
  Antisense oligonucleotide
ggggtctcgttttaaggct

SEQ ID NO: 374 - DNA, 20 bp, Artificial Sequence
  Antisense oligonucleotide
ccaggggtctcgttttaagg

SEQ ID NO: 375 - DNA, 16 bp, Artificial Sequence
  Antisense oligonucleotide
ggggtctcgttttaag

SEQ ID NO: 376 - DNA, 17 bp, Artificial Sequence
  Antisense oligonucleotide
aggggtctcgttttaag

SEQ ID NO: 377 - DNA, 18 bp, Artificial Sequence
  Antisense oligonucleotide
caggggtctcgttttaag

SEQ ID NO: 378 - DNA, 19 bp, Artificial Sequence
  Antisense oligonucleotide
ccaggggtctcgttttaag

SEQ ID NO: 379 - DNA, 17 bp, Artificial Sequence
  Antisense oligonucleotide
caggggtctcgttttaa

SEQ ID NO: 380 - DNA, 18 bp, Artificial Sequence
  Antisense oligonucleotide
ccaggggtctcgttttaa

SEQ ID NO: 381 - DNA, 19 bp, Artificial Sequence
  Antisense oligonucleotide
cccaggggtctcgttttaa

SEQ ID NO: 382 - DNA, 20 bp, Artificial Sequence
  Antisense oligonucleotide
ccccaggggtctcgttttaa

SEQ ID NO: 383 - DNA, 20 bp, Artificial Sequence
  Antisense oligonucleotide
tgcacatttgataaattttg

SEQ ID NO: 384 - DNA, 20 bp, Artificial Sequence
  Antisense oligonucleotide
gtgcacatttgataaatttt

SEQ ID NO: 385 - DNA, 19 bp, Artificial Sequence
  Antisense oligonucleotide
gtgcacatttgataaattt

SEQ ID NO: 386 - DNA, 18 bp, Artificial Sequence
  Antisense oligonucleotide
gtgcacatttgataaatt

SEQ ID NO: 387 - DNA, 19 bp, Artificial Sequence
  Antisense oligonucleotide
cgtgcacatttgataaatt

SEQ ID NO: 388 - DNA, 18 bp, Artificial Sequence
  Antisense oligonucleotide
cgtgcacatttgataaat

SEQ ID NO: 389 - DNA, 19 bp, Artificial Sequence
  Antisense oligonucleotide
acgtgcacatttgataaat

SEQ ID NO: 390 - DNA, 20 bp, Artificial Sequence
  Antisense oligonucleotide
cacgtgcacatttgataaat

SEQ ID NO: 391 - DNA, 17 bp, Artificial Sequence
  Antisense oligonucleotide
cgtgcacatttgataaa

SEQ ID NO: 392 - DNA, 18 bp, Artificial Sequence
  Antisense oligonucleotide
acgtgcacatttgataaa

SEQ ID NO: 393 - DNA, 19 bp, Artificial Sequence
  Antisense oligonucleotide
cacgtgcacatttgataaa

SEQ ID NO: 394 - DNA, 20 bp, Artificial Sequence
  Antisense oligonucleotide
acacgtgcacatttgataaa

SEQ ID NO: 395 - DNA, 17 bp, Artificial Sequence
  Antisense oligonucleotide
acgtgcacatttgataa

SEQ ID NO: 396 - DNA, 18 bp, Artificial Sequence
  Antisense oligonucleotide
cacgtgcacatttgataa

SEQ ID NO: 397 - DNA, 20 bp, Artificial Sequence
  Antisense oligonucleotide
cacacgtgcacatttgataa

SEQ ID NO: 398 - DNA, 19 bp, Artificial Sequence
  Antisense oligonucleotide
cacacgtgcacatttgata

SEQ ID NO: 399 - DNA, 18 bp, Artificial Sequence
  Antisense oligonucleotide
cacacgtgcacatttgat

SEQ ID NO: 400 - DNA, 16 bp, Artificial Sequence
  Antisense oligonucleotide
cggtggacacagcgtg

SEQ ID NO: 401 - DNA, 15 bp, Artificial Sequence
  Antisense oligonucleotide
gaggacgtcaagccg

SEQ ID NO: 402 - DNA, 16 bp, Artificial Sequence
  Antisense oligonucleotide
ggaggacgtcaagccg

SEQ ID NO: 403 - DNA, 16 bp, Artificial Sequence
  Antisense oligonucleotide
cggaggacgtcaagcc

SEQ ID NO: 404 - DNA, 16 bp, Artificial Sequence
  Antisense oligonucleotide
ccggaggacgtcaagc

SEQ ID NO: 405 - DNA, 16 bp, Artificial Sequence
  Antisense oligonucleotide
agagcgggatcctcca

SEQ ID NO: 406 - DNA, 17 bp, Artificial Sequence
  Antisense oligonucleotide
cacagagcgggatcctc

SEQ ID NO: 407 - DNA, 16 bp, Artificial Sequence
  Antisense oligonucleotide
gcacagagcgggatcc

SEQ ID NO: 408 - DNA, 17 bp, Artificial Sequence
  Antisense oligonucleotide
agggcacagagcgggat

SEQ ID NO: 409 - DNA, 18 bp, Artificial Sequence
  Antisense oligonucleotide
ctctgtggtcatagaaaa

SEQ ID NO: 410 - DNA, 19 bp, Artificial Sequence
  Antisense oligonucleotide
gctctgtggtcatagaaaa

SEQ ID NO: 411 - DNA, 20 bp, Artificial Sequence
  Antisense oligonucleotide
agctctgtggtcatagaaaa

SEQ ID NO: 412 - DNA, 18 bp, Artificial Sequence
  Antisense oligonucleotide
gctctgtggtcatagaaa

SEQ ID NO: 413 - DNA, 19 bp, Artificial Sequence
  Antisense oligonucleotide
agctctgtggtcatagaaa

SEQ ID NO: 414 - DNA, 20 bp, Artificial Sequence
  Antisense oligonucleotide
gagctctgtggtcatagaaa

SEQ ID NO: 415 - DNA, 17 bp, Artificial Sequence
  Antisense oligonucleotide
gctctgtggtcatagaa

SEQ ID NO: 416 - DNA, 18 bp, Artificial Sequence
  Antisense oligonucleotide
agctctgtggtcatagaa

SEQ ID NO: 417 - DNA, 19 bp, Artificial Sequence
  Antisense oligonucleotide
gagctctgtggtcatagaa

SEQ ID NO: 418 - DNA, 20 bp, Artificial Sequence
  Antisense oligonucleotide
ggagctctgtggtcatagaa

SEQ ID NO: 419 - DNA, 18 bp, Artificial Sequence
  Antisense oligonucleotide
gagctctgtggtcataga

SEQ ID NO: 420 - DNA, 17 bp, Artificial Sequence
  Antisense oligonucleotide
ggagctctgtggtcata

SEQ ID NO: 421 - DNA, 18 bp, Artificial Sequence
  Antisense oligonucleotide
cggagctctgtggtcata

SEQ ID NO: 422 - DNA, 17 bp, Artificial Sequence
  Antisense oligonucleotide
cggagctctgtggtcat

SEQ ID NO: 423 - DNA, 18 bp, Artificial Sequence
  Antisense oligonucleotide
caggtgaaggaaggccag

SEQ ID NO: 424 - DNA, 18 bp, Artificial Sequence
  Antisense oligonucleotide
ccaggtgaaggaaggcca

SEQ ID NO: 425 - DNA, 19 bp, Artificial Sequence
  Antisense oligonucleotide
cccaggtgaaggaaggcca

SEQ ID NO: 426 - DNA, 18 bp, Artificial Sequence
  Antisense oligonucleotide
cccaggtgaaggaaggcc

SEQ ID NO: 427 - DNA, 18 bp, Artificial Sequence
  Antisense oligonucleotide
ccccaggtgaaggaaggc

SEQ ID NO: 428 - DNA, 17 bp, Artificial Sequence
  Antisense oligonucleotide
ctgtgctgaagatgggc

SEQ ID NO: 429 - DNA, 17 bp, Artificial Sequence
  Antisense oligonucleotide
cctgtgctgaagatggg

SEQ ID NO: 430 - DNA, 16 bp, Artificial Sequence
  Antisense oligonucleotide
attgcggcacgggctg

SEQ ID NO: 431 - DNA, 19 bp, Artificial Sequence
  Antisense oligonucleotide
attttacttatccccagcc

SEQ ID NO: 432 - DNA, 20 bp, Artificial Sequence
  Antisense oligonucleotide
cattttacttatccccagcc

SEQ ID NO: 433 - DNA, 17 bp, Artificial Sequence
  Antisense oligonucleotide
ttttacttatccccagc

SEQ ID NO: 434 - DNA, 18 bp, Artificial Sequence
  Antisense oligonucleotide
attttacttatccccagc

SEQ ID NO: 435 - DNA, 18 bp, Artificial Sequence
  Antisense oligonucleotide
cattttacttatccccag

SEQ ID NO: 436 - DNA, 19 bp, Artificial Sequence
  Antisense oligonucleotide
ccattttacttatccccag

SEQ ID NO: 437 - DNA, 18 bp, Artificial Sequence
  Antisense oligonucleotide
ccattttacttatcccca

SEQ ID NO: 438 - DNA, 17 bp, Artificial Sequence
  Antisense oligonucleotide
ccattttacttatcccc

SEQ ID NO: 439 - DNA, 17 bp, Artificial Sequence
  Antisense oligonucleotide
ctctgtagtttgttctc

SEQ ID NO: 440 - DNA, 17 bp, Artificial Sequence
  Antisense oligonucleotide
actgcaccgggacacag

SEQ ID NO: 441 - DNA, 16 bp, Artificial Sequence
  Antisense oligonucleotide
gcccgctagaagcccc

SEQ ID NO: 442 - DNA, 17 bp, Artificial Sequence
  Antisense oligonucleotide
acctacctcatcaccac

SEQ ID NO: 443 - DNA, 17 bp, Artificial Sequence
  Antisense oligonucleotide
acacctacctcatcacc

SEQ ID NO: 444 - DNA, 18 bp, Artificial Sequence
  Antisense oligonucleotide
aacacctacctcatcacc

SEQ ID NO: 445 - DNA, 19 bp, Artificial Sequence
  Antisense oligonucleotide
aaacacctacctcatcacc

SEQ ID NO: 446 - DNA, 18 bp, Artificial Sequence
  Antisense oligonucleotide
aaacacctacctcatcac

SEQ ID NO: 447 - DNA, 19 bp, Artificial Sequence
  Antisense oligonucleotide
caaacacctacctcatcac

SEQ ID NO: 448 - DNA, 20 bp, Artificial Sequence
  Antisense oligonucleotide
gcaaacacctacctcatcac

SEQ ID NO: 449 - DNA, 18 bp, Artificial Sequence
  Antisense oligonucleotide
caaacacctacctcatca

SEQ ID NO: 450 - DNA, 19 bp, Artificial Sequence
  Antisense oligonucleotide
gcaaacacctacctcatca

SEQ ID NO: 451 - DNA, 17 bp, Artificial Sequence
  Antisense oligonucleotide
caaacacctacctcatc

SEQ ID NO: 452 - DNA, 18 bp, Artificial Sequence
  Antisense oligonucleotide
gcaaacacctacctcatc

SEQ ID NO: 453 - DNA, 17 bp, Artificial Sequence
  Antisense oligonucleotide
gcaaacacctacctcat

SEQ ID NO: 454 - DNA, 16 bp, Artificial Sequence
  Antisense oligonucleotide
cctacatgggggcttg

SEQ ID NO: 455 - DNA, 16 bp, Artificial Sequence
  Antisense oligonucleotide
gcctacatgggggctt

SEQ ID NO: 456 - DNA, 18 bp, Artificial Sequence
  Antisense oligonucleotide
ttgggagagaaccttcag

SEQ ID NO: 457 - DNA, 19 bp, Artificial Sequence
  Antisense oligonucleotide
attgggagagaaccttcag

SEQ ID NO: 458 - DNA, 20 bp, Artificial Sequence
  Antisense oligonucleotide
aattgggagagaaccttcag

SEQ ID NO: 459 - DNA, 18 bp, Artificial Sequence
  Antisense oligonucleotide
attgggagagaaccttca

SEQ ID NO: 460 - DNA, 19 bp, Artificial Sequence
  Antisense oligonucleotide
aattgggagagaaccttca

SEQ ID NO: 461 - DNA, 20 bp, Artificial Sequence
  Antisense oligonucleotide
caattgggagagaaccttca

SEQ ID NO: 462 - DNA, 19 bp, Artificial Sequence
  Antisense oligonucleotide
caattgggagagaaccttc

SEQ ID NO: 463 - DNA, 18 bp, Artificial Sequence
  Antisense oligonucleotide
caattgggagagaacctt

SEQ ID NO: 464 - DNA, 17 bp, Artificial Sequence
  Antisense oligonucleotide
caattgggagagaacct

SEQ ID NO: 465 - DNA, 18 bp, Artificial Sequence
  Antisense oligonucleotide
aaagcatctgtgggcatg

SEQ ID NO: 466 - DNA, 18 bp, Artificial Sequence
  Antisense oligonucleotide
ccaaagcatctgtgggca

SEQ ID NO: 467 - DNA, 17 bp, Artificial Sequence
  Antisense oligonucleotide
ccatcactccaaagcat

SEQ ID NO: 468 - DNA, 18 bp, Artificial Sequence
  Antisense oligonucleotide
aaaggagagtcgtgcctg

SEQ ID NO: 469 - DNA, 17 bp, Artificial Sequence
  Antisense oligonucleotide
aaaggagagtcgtgcct

SEQ ID NO: 470 - DNA, 16 bp, Artificial Sequence
  Antisense oligonucleotide
aaaggagagtcgtgcc

SEQ ID NO: 471 - DNA, 17 bp, Artificial Sequence
  Antisense oligonucleotide
gaaaggagagtcgtgcc

SEQ ID NO: 472 - DNA, 19 bp, Artificial Sequence
  Antisense oligonucleotide
tggaaaggagagtcgtgcc

SEQ ID NO: 473 - DNA, 20 bp, Artificial Sequence
  Antisense oligonucleotide
ctggaaaggagagtcgtgcc

SEQ ID NO: 474 - DNA, 16 bp, Artificial Sequence
  Antisense oligonucleotide
gaaaggagagtcgtgc

SEQ ID NO: 475 - DNA, 17 bp, Artificial Sequence
  Antisense oligonucleotide
ggaaaggagagtcgtgc

SEQ ID NO: 476 - DNA, 18 bp, Artificial Sequence
  Antisense oligonucleotide
tggaaaggagagtcgtgc

SEQ ID NO: 477 - DNA, 19 bp, Artificial Sequence
  Antisense oligonucleotide
ctggaaaggagagtcgtgc

SEQ ID NO: 478 - DNA, 20 bp, Artificial Sequence
  Antisense oligonucleotide
cctggaaaggagagtcgtgc

SEQ ID NO: 479 - DNA, 17 bp, Artificial Sequence
  Antisense oligonucleotide
tggaaaggagagtcgtg

SEQ ID NO: 480 - DNA, 18 bp, Artificial Sequence
  Antisense oligonucleotide
ctggaaaggagagtcgtg

SEQ ID NO: 481 - DNA, 19 bp, Artificial Sequence
  Antisense oligonucleotide
cctggaaaggagagtcgtg

SEQ ID NO: 482 - DNA, 17 bp, Artificial Sequence
  Antisense oligonucleotide
ctggaaaggagagtcgt

SEQ ID NO: 483 - DNA, 18 bp, Artificial Sequence
  Antisense oligonucleotide
cctggaaaggagagtcgt

SEQ ID NO: 484 - DNA, 17 bp, Artificial Sequence
  Antisense oligonucleotide
cctggaaaggagagtcg

SEQ ID NO: 485 - DNA, 18 bp, Artificial Sequence
  Antisense oligonucleotide
ctacaacaaagcccgagg

SEQ ID NO: 486 - DNA, 20 bp, Artificial Sequence
  Antisense oligonucleotide
ttctacaacaaagcccgagg

SEQ ID NO: 487 - DNA, 17 bp, Artificial Sequence
  Antisense oligonucleotide
ctacaacaaagcccgag

SEQ ID NO: 488 - DNA, 18 bp, Artificial Sequence
  Antisense oligonucleotide
tctacaacaaagcccgag

SEQ ID NO: 489 - DNA, 19 bp, Artificial Sequence
  Antisense oligonucleotide
ttctacaacaaagcccgag

SEQ ID NO: 490 - DNA, 20 bp, Artificial Sequence
  Antisense oligonucleotide
tttctacaacaaagcccgag

SEQ ID NO: 491 - DNA, 17 bp, Artificial Sequence
  Antisense oligonucleotide
tctacaacaaagcccga

SEQ ID NO: 492 - DNA, 19 bp, Artificial Sequence
  Antisense oligonucleotide
tttctacaacaaagcccga

SEQ ID NO: 493 - DNA, 20 bp, Artificial Sequence
  Antisense oligonucleotide
gtttctacaacaaagcccga

SEQ ID NO: 494 - DNA, 16 bp, Artificial Sequence
  Antisense oligonucleotide
tctacaacaaagcccg

SEQ ID NO: 495 - DNA, 19 bp, Artificial Sequence
  Antisense oligonucleotide
gtttctacaacaaagcccg

SEQ ID NO: 496 - DNA, 18 bp, Artificial Sequence
  Antisense oligonucleotide
gtttctacaacaaagccc

SEQ ID NO: 497 - DNA, 19 bp, Artificial Sequence
  Antisense oligonucleotide
tgtttctacaacaaagccc

SEQ ID NO: 498 - DNA, 20 bp, Artificial Sequence
  Antisense oligonucleotide
ttgtttctacaacaaagccc

SEQ ID NO: 499 - DNA, 18 bp, Artificial Sequence
  Antisense oligonucleotide
tgtttctacaacaaagcc

SEQ ID NO: 500 - DNA, 19 bp, Artificial Sequence
  Antisense oligonucleotide
ttgtttctacaacaaagcc

SEQ ID NO: 501 - DNA, 20 bp, Artificial Sequence
  Antisense oligonucleotide
attgtttctacaacaaagcc

SEQ ID NO: 502 - DNA, 18 bp, Artificial Sequence
  Antisense oligonucleotide
ttgtttctacaacaaagc

SEQ ID NO: 503 - DNA, 19 bp, Artificial Sequence
  Antisense oligonucleotide
attgtttctacaacaaagc

SEQ ID NO: 504 - DNA, 20 bp, Artificial Sequence
  Antisense oligonucleotide
cattgtttctacaacaaagc

SEQ ID NO: 505 - DNA, 18 bp, Artificial Sequence
  Antisense oligonucleotide
attgtttctacaacaaag

SEQ ID NO: 506 - DNA, 19 bp, Artificial Sequence
  Antisense oligonucleotide
cattgtttctacaacaaag

SEQ ID NO: 507 - DNA, 20 bp, Artificial Sequence
  Antisense oligonucleotide
ccattgtttctacaacaaag

SEQ ID NO: 508 - DNA, 19 bp, Artificial Sequence
  Antisense oligonucleotide
ccattgtttctacaacaaa

SEQ ID NO: 509 - DNA, 20 bp, Artificial Sequence
  Antisense oligonucleotide
gccattgtttctacaacaaa

SEQ ID NO: 510 - DNA, 18 bp, Artificial Sequence
  Antisense oligonucleotide
ccattgtttctacaacaa

SEQ ID NO: 511 - DNA, 19 bp, Artificial Sequence
  Antisense oligonucleotide
gccattgtttctacaacaa

SEQ ID NO: 512 - DNA, 20 bp, Artificial Sequence
  Antisense oligonucleotide
ggccattgtttctacaacaa

SEQ ID NO: 513 - DNA, 18 bp, Artificial Sequence
  Antisense oligonucleotide
gccattgtttctacaaca

SEQ ID NO: 514 - DNA, 19 bp, Artificial Sequence
  Antisense oligonucleotide
ggccattgtttctacaaca

SEQ ID NO: 515 - DNA, 18 bp, Artificial Sequence
  Antisense oligonucleotide
ggccattgtttctacaac

SEQ ID NO: 516 - DNA, 19 bp, Artificial Sequence
  Antisense oligonucleotide
tttcagatgccaagacaca

SEQ ID NO: 517 - DNA, 20 bp, Artificial Sequence
  Antisense oligonucleotide
atttcagatgccaagacaca

SEQ ID NO: 518 - DNA, 19 bp, Artificial Sequence
  Antisense oligonucleotide
atttcagatgccaagacac

SEQ ID NO: 519 - DNA, 20 bp, Artificial Sequence
  Antisense oligonucleotide
catttcagatgccaagacac

SEQ ID NO: 520 - DNA, 18 bp, Artificial Sequence
  Antisense oligonucleotide
atttcagatgccaagaca

SEQ ID NO: 521 - DNA, 19 bp, Artificial Sequence
  Antisense oligonucleotide
catttcagatgccaagaca

SEQ ID NO: 522 - DNA, 19 bp, Artificial Sequence
  Antisense oligonucleotide
gcatttcagatgccaagac

SEQ ID NO: 523 - DNA, 18 bp, Artificial Sequence
  Antisense oligonucleotide
gtagcctgcatttcagat

SEQ ID NO: 524 - DNA, 16 bp, Artificial Sequence
  Antisense oligonucleotide
tacctgcggtagttct

SEQ ID NO: 525 - DNA, 17 bp, Artificial Sequence
  Antisense oligonucleotide
ctacctgcggtagttct

SEQ ID NO: 526 - DNA, 16 bp, Artificial Sequence
  Antisense oligonucleotide
ctacctgcggtagttc

SEQ ID NO: 527 - DNA, 17 bp, Artificial Sequence
  Antisense oligonucleotide
cctacctgcggtagttc

SEQ ID NO: 528 - DNA, 15 bp, Artificial Sequence
  Antisense oligonucleotide
ctacctgcggtagtt

SEQ ID NO: 529 - DNA, 16 bp, Artificial Sequence
  Antisense oligonucleotide
cctacctgcggtagtt

SEQ ID NO: 530 - DNA, 17 bp, Artificial Sequence
  Antisense oligonucleotide
gcctacctgcggtagtt

SEQ ID NO: 531 - DNA, 15 bp, Artificial Sequence
  Antisense oligonucleotide
gcctacctgcggtag

SEQ ID NO: 532 - DNA, 16 bp, Artificial Sequence
  Antisense oligonucleotide
cgcctacctgcggtag

SEQ ID NO: 533 - DNA, 18 bp, Artificial Sequence
  Antisense oligonucleotide
ttttggagaagcctgggg

SEQ ID NO: 534 - DNA, 18 bp, Artificial Sequence
  Antisense oligonucleotide
gttttggagaagcctggg

SEQ ID NO: 535 - DNA, 20 bp, Artificial Sequence
  Antisense oligonucleotide
ccgttttggagaagcctggg

SEQ ID NO: 536 - DNA, 18 bp, Artificial Sequence
  Antisense oligonucleotide
cgttttggagaagcctgg

SEQ ID NO: 537 - DNA, 20 bp, Artificial Sequence
  Antisense oligonucleotide
cccgttttggagaagcctgg

SEQ ID NO: 538 - DNA, 17 bp, Artificial Sequence
  Antisense oligonucleotide
cgttttggagaagcctg

SEQ ID NO: 539 - DNA, 18 bp, Artificial Sequence
  Antisense oligonucleotide
ccgttttggagaagcctg

SEQ ID NO: 540 - DNA, 19 bp, Artificial Sequence
  Antisense oligonucleotide
cccgttttggagaagcctg

SEQ ID NO: 541 - DNA, 20 bp, Artificial Sequence
  Antisense oligonucleotide
gcccgttttggagaagcctg

SEQ ID NO: 542 - DNA, 16 bp, Artificial Sequence
  Antisense oligonucleotide
cgttttggagaagcct

SEQ ID NO: 543 - DNA, 17 bp, Artificial Sequence
  Antisense oligonucleotide
ccgttttggagaagcct

SEQ ID NO: 544 - DNA, 18 bp, Artificial Sequence
  Antisense oligonucleotide
cccgttttggagaagcct

SEQ ID NO: 545 - DNA, 19 bp, Artificial Sequence
  Antisense oligonucleotide
gcccgttttggagaagcct

SEQ ID NO: 546 - DNA, 17 bp, Artificial Sequence
  Antisense oligonucleotide
cccgttttggagaagcc

SEQ ID NO: 547 - DNA, 18 bp, Artificial Sequence
  Antisense oligonucleotide
gcccgttttggagaagcc

SEQ ID NO: 548 - DNA, 19 bp, Artificial Sequence
  Antisense oligonucleotide
agcccgttttggagaagcc

SEQ ID NO: 549 - DNA, 17 bp, Artificial Sequence
  Antisense oligonucleotide
gcccgttttggagaagc

SEQ ID NO: 550 - DNA, 18 bp, Artificial Sequence
  Antisense oligonucleotide
agcccgttttggagaagc

SEQ ID NO: 551 - DNA, 19 bp, Artificial Sequence
  Antisense oligonucleotide
cagcccgttttggagaagc

SEQ ID NO: 552 - DNA, 17 bp, Artificial Sequence
  Antisense oligonucleotide
agcccgttttggagaag

SEQ ID NO: 553 - DNA, 18 bp, Artificial Sequence
  Antisense oligonucleotide
cagcccgttttggagaag

SEQ ID NO: 554 - DNA, 17 bp, Artificial Sequence
  Antisense oligonucleotide
cagcccgttttggagaa

SEQ ID NO: 555 - DNA, 20 bp, Artificial Sequence
  Antisense oligonucleotide
ccccagcccgttttggagaa

SEQ ID NO: 556 - DNA, 17 bp, Artificial Sequence
  Antisense oligonucleotide
ttcagggcaccagattc

SEQ ID NO: 557 - DNA, 18 bp, Artificial Sequence
  Antisense oligonucleotide
tttcagggcaccagattc

SEQ ID NO: 558 - DNA, 18 bp, Artificial Sequence
  Antisense oligonucleotide
ctttcagggcaccagatt

SEQ ID NO: 559 - DNA, 16 bp, Artificial Sequence
  Antisense oligonucleotide
gtgccgcttaacaaac

SEQ ID NO: 560 - DNA, 17 bp, Artificial Sequence
  Antisense oligonucleotide
agtgccgcttaacaaac

SEQ ID NO: 561 - DNA, 19 bp, Artificial Sequence
  Antisense oligonucleotide
tgagtgccgcttaacaaac

SEQ ID NO: 562 - DNA, 16 bp, Artificial Sequence
  Antisense oligonucleotide
agtgccgcttaacaaa

SEQ ID NO: 563 - DNA, 18 bp, Artificial Sequence
  Antisense oligonucleotide
tgagtgccgcttaacaaa

SEQ ID NO: 564 - DNA, 17 bp, Artificial Sequence
  Antisense oligonucleotide
tgagtgccgcttaacaa

SEQ ID NO: 565 - DNA, 16 bp, Artificial Sequence
  Antisense oligonucleotide
tgagtgccgcttaaca

SEQ ID NO: 566 - DNA, 18 bp, Artificial Sequence
  Antisense oligonucleotide
acagatggcgtgtgcatg

SEQ ID NO: 567 - DNA, 17 bp, Artificial Sequence
  Antisense oligonucleotide
tacacagatggcgtgtg

SEQ ID NO: 568 - DNA, 18 bp, Artificial Sequence
  Antisense oligonucleotide
ttacacagatggcgtgtg

SEQ ID NO: 569 - DNA, 17 bp, Artificial Sequence
  Antisense oligonucleotide
ttacacagatggcgtgt

SEQ ID NO: 570 - DNA, 18 bp, Artificial Sequence
  Antisense oligonucleotide
gttacacagatggcgtgt

SEQ ID NO: 571 - DNA, 20 bp, Artificial Sequence
  Antisense oligonucleotide
atgtattgtgtgttacatgg

SEQ ID NO: 572 - DNA, 19 bp, Artificial Sequence
  Antisense oligonucleotide
atgtattgtgtgttacatg

SEQ ID NO: 573 - DNA, 20 bp, Artificial Sequence
  Antisense oligonucleotide
catgtattgtgtgttacatg

SEQ ID NO: 574 - DNA, 18 bp, Artificial Sequence
  Antisense oligonucleotide
atgtattgtgtgttacat

SEQ ID NO: 575 - DNA, 18 bp, Artificial Sequence
  Antisense oligonucleotide
acccgtgctgtttattta

SEQ ID NO: 576 - DNA, 17 bp, Artificial Sequence
  Antisense oligonucleotide
acccgtgctgtttattt

SEQ ID NO: 577 - DNA, 18 bp, Artificial Sequence
  Antisense oligonucleotide
cacccgtgctgtttattt

(19) United States
(12) Patent Application Publication
(10) Pub. No.: US 2022/0042011 A1
(43) Pub. Date: Feb. 10, 2022

(54) ANTISENSE OLIGONUCLEOTIDES TARGETING CARD9

(71) Applicants: Hoffmann-La Roche Inc., Little Falls, NJ (US); Boehringer Ingelheim International GmbH, Ingelheim am Rhein (DE)

(72) Inventors: Jay FINE, Ridgefield, CT (US); Mouhamadou Lamine MBOW, Ridgefield, CT (US); Joe Adam WAHLE, Ridgefield, CT (US); Fei SHEN, Ridgefield, CT (US); Elliott Sanford KLEIN, Ridgefield, CT (US); Kristina Mary SAI, Ridgefield, CT (US); Peter HAGEDORN, Horsholm (DK); Anja MOELHART HOEG, Horsholm (DK)

(73) Assignees: Hoffmann-La Roche Inc., Little Falls, NJ (US); Boehringer Ingelheim International GmbH, Ingelheim am Rhein (DE)

(21) Appl. No.: 17/311,175

(22) PCT Filed: Dec. 20, 2019
(86) PCT No.: PCT/EP2019/086725

§ 371 Date: Jun. 4, 2021

Related U.S. Application Data
(60) Provisional application No. 62/784,285, filed on Dec. 21, 2018.
(60) Provisional application No. 62/832,207, filed on Apr. 10, 2019.

(51) Int. Cl. C12N 15/113

(52) U.S. Cl. CPC C12N 15/113; C12N 2310/11; C12N 2310/341; C12N 2320/30; C12N 2310/315; C12N 2310/3341; C12N 2310/351; C12N 2310/3231

(57) ABSTRACT

The present invention relates to antisense LNA oligonucleotides (oligomers) complementary to CARD9 pre-mRNA intron and exon sequences, which are capable of inhibiting the expression of CARD9 protein. Inhibition of CARD9 expression is beneficial for a range of medical disorders including inflammatory bowel disease, pancreatitis, IgA nephropathy, primary sclerosing cholangitis, cardiovascular disease, cancer and diabetes.

### REFERENCE TO SEQUENCE LISTING SUBMITTED ELECTRONICALLY

The content of the electronically-submitted sequence listing (Name: P35118-WO 02-0499-WO Sequence_Listing_CARD9.txt; Size: 178,721 bytes; and Date of Creation: Dec. 16, 2019) submitted in this application is herein incorporated by reference in its entirety.

### FIELD OF INVENTION

The present invention relates to antisense LNA oligonucleotides (oligomers) complementary to CARD9 pre-mRNA sequences, which are capable of inhibiting the expression of CARD9. Inhibition of CARD9 expression is beneficial for a range of medical disorders including inflammatory bowel disease (such as Crohn's disease and ulcerative colitis), pancreatitis, IgA nephropathy, primary sclerosing cholangitis, cardiovascular disease, cancer and diabetes.

### BACKGROUND

CARD9 (Caspase recruitment domain-containing protein 9) is a central component of anti-fungal innate immune signaling via C-type lectin receptors. It is a member of the CARD family which plays an important role in innate immune response by the activation of NF-κB.

CARD9 mediates pro-inflammatory cytokine production, including TNFα, IL-6, and IL-1β, thereby regulating the responses of Th1 and Th17 cells.

CARD9 has been associated with many diseases and disorders. For example, CARD9 expression has been associated with cardiovascular disease, autoimmune disease, cancer and obesity (Zhong et al. Cell Death and Disease (2018) 9:52).

Further, CARD9 has been identified as a gene associated with the risk of inflammatory bowel disease (IBD), ankylosing spondylitis, primary sclerosing cholangitis, and IgA nephropathy (Cao et al., Immunity 2015 Oct. 20; 43(4): 715-726).

Small molecule inhibitors have been used to directly target the CARD9 to determine the feasibility of using small using small-molecule inhibitors to recapitulate the antiinflammatory 30 function of CARD9 mutations associated with protection from IBD (Leshchiner et al., Proc Natl Acad Sci USA. 2017 Oct. 24; 114(43): 11392-11397).

Yamamoto-Furusho showed that expression of CARD9 can differently distinguish active and remission ulcerative colitis (UC). Therefore, CARD9 was proposed as target for in UC patients (Journal of Inflammation (2018) 15:13).

Further, it was shown that CARD9 expression is upregulated in severe acute pancreatitis (SAP) patients. Small interfering RNAs (siRNAs) were used to reduce the levels of CARD9 expression in sodium taurocholate-stimulated SAP rats. When compared to the untreated group, the cohort that received the siRNA treatment demonstrated a significant reduction in pancreatic injury, neutrophil infiltration, myeloperoxidase activity and pro-inflammatory cytokines. Therefore, CARD9 was suggested as target for the treatment of acute pancreatitis (Yang et al., J Cell Mol Med. 2016; 21(6):1085-1093).

Moreover, CARD9 was proposed as target for the treatment of neutrophilic dermatoses (Tartey et al., The Journal of Immunology Sep. 15, 2018, 201 (6) 1639-1644).

We have analyzed a large number of LNA gapmers targeting human CARD9 and identified target sites, oligonucleotide sequences and antisense compounds which are potent and effective to inhibitors of CARD9 expression.

### OBJECTIVE OF THE INVENTION

The present invention identifies regions of the CARD9 transcript (CARD9) for antisense inhibition in vitro or in vivo, and provides for antisense oligonucleotides, including LNA gapmer oligonucleotides, which target these regions of the CARD9 premRNA or mature mRNA. The present invention identifies oligonucleotides which inhibit human CARD9 which are useful in the treatment of a range of medical disorders including inflammatory bowel disease, pancreatitis, IgA nephropathy, primary sclerosing cholangitis, cardiovascular disease, cancer and diabetes.

### STATEMENT OF THE INVENTION

The invention provides for an antisense oligonucleotide, 10-30 nucleotides in length, targeting a mammalian CARD9 (Caspase recruitment domain-containing protein 9) target nucleic acid, wherein the antisense oligonucleotide is capable of inhibiting the expression of mammalian CARD9 in a cell which is expressing mammalian CARD9. The mammalian CARD9 target nucleic acid may be, e.g., a human, monkey, mouse or porcine CARD9 target nucleic acid.

Accordingly, the invention provides for an antisense oligonucleotide, 10-30 nucleotides in length, targeting a human CARD9 target nucleic acid, wherein the antisense oligonucleotide is capable of inhibiting the expression of human CARD9 in a cell which is expressing human CARD9.

The invention provides for an antisense oligonucleotide, 10-30 nucleotides in length, targeting a mammalian (such as a human, monkey, mouse or porcine) CARD9 target nucleic acid, wherein said antisense oligonucleotide comprises a contiguous nucleotide sequence 10-30 nucleotides in length, wherein the contiguous nucleotide sequence is at least 90% complementary, such as fully complementary, to a sequence selected from the group consisting of SEQ ID NO 1, 2, 3, 4, 5, 7, 8 and 9.

The invention provides for an antisense oligonucleotide, 10-30 nucleotides in length, targeting a human CARD9 target nucleic acid, wherein said antisense oligonucleotide comprises a contiguous nucleotide sequence 10-30 nucleotides in length, wherein the contiguous nucleotide sequence is at least 90% complementary, such as fully complementary to SEQ ID NO 1.

The invention provides for an antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary, to SEQ ID NO 1 wherein the antisense oligonucleotide is capable of inhibiting the expression of human CARD9 in a cell which is expressing human CARD9.

The invention provides for an LNA antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary, to SEQ ID NO 1, wherein the antisense oligonucleotide is capable of inhibiting the expression of human CARD9 in a cell which is expressing human CARD9.

The invention provides for a gapmer antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary, to SEQ ID NO 1, wherein the antisense oligonucleotide is capable of inhibiting the expression of human CARD9 in a cell which is expressing human CARD9.

The invention provides for an LNA gapmer antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary, to SEQ ID NO 1 wherein the antisense oligonucleotide is capable of inhibiting the expression of human CARD9 in a cell which is expressing human CARD9.

The invention provides for an antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary, to a sequence selected from the group consisting of SEQ ID NO 10 to SEQ ID NO: 69, wherein the antisense oligonucleotide is capable of inhibiting the expression of human CARD9 in a cell which is expressing human CARD9.

The invention provides for an LNA antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary, to a sequence selected from the group consisting of SEQ ID NO 10 to SEQ ID NO: 69, wherein the antisense oligonucleotide is capable of inhibiting the expression of human CARD9 in a cell which is expressing human CARD9.

The invention provides for a gapmer antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary to a sequence selected from the group consisting of SEQ ID NO 10 to SEQ ID NO: 69, wherein the antisense oligonucleotide is capable of inhibiting the expression of human CARD9 in a cell which is expressing human CARD9.

The invention provides for an LNA gapmer antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary, to a sequence selected from the group consisting of SEQ ID NO 10 to SEQ ID NO: 69, wherein the antisense oligonucleotide is capable of inhibiting the expression of human CARD9 in a cell which is expressing human CARD9.

The oligonucleotide of the invention as referred to or claimed herein may be in the form of a pharmaceutically acceptable salt.

The invention provides for a conjugate comprising the oligonucleotide according to the invention, and at least one conjugate moiety covalently attached to said oligonucleotide.

The invention provides for a pharmaceutical composition comprising the oligonucleotide or conjugate of the invention and a pharmaceutically acceptable diluent, solvent, carrier, salt and/or adjuvant.

The invention provides for an in vivo or in vitro method for modulating CARD9 expression in a target cell which is expressing CARD9, said method comprising administering an oligonucleotide or conjugate or pharmaceutical composition of the invention in an effective amount to said cell.

The invention provides for a method for treating or preventing a disease comprising administering a therapeutically or prophylactically effective amount of an oligonucleotide, conjugate or the pharmaceutical composition of the invention to a subject suffering from or susceptible to the disease.

In some embodiments, the disease is selected from the group consisting of inflammatory bowel disease, pancreatitis, IgA nephropathy, primary sclerosing cholangitis, cardiovascular disease, cancer and diabetes.

The invention provides for the oligonucleotide, conjugate or the pharmaceutical composition of the invention for use in medicine.

The invention provides for the oligonucleotide, conjugate or the pharmaceutical composition of the invention for use in the treatment or prevention of a disease selected from the group consisting of inflammatory bowel disease, pancreatitis, IgA nephropathy, primary sclerosing cholangitis, cardiovascular disease, cancer and diabetes.

The invention provides for the use of the oligonucleotide, conjugate or the pharmaceutical composition of the invention, for the preparation of a medicament for treatment or prevention of a disease selected from the group consisting of inflammatory bowel disease, pancreatitis, IgA nephropathy, primary sclerosing cholangitis, cardiovascular disease, cancer and diabetes.

### Definitions

Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides or nucleotides.

Antisense Oligonucleotides

The term “Antisense oligonucleotide” as used herein is defined as oligonucleotides capable of modulating expression of a target gene by hybridizing to a target nucleic acid, in particular to a contiguous sequence on a target nucleic acid. The antisense oligonucleotides are not essentially double stranded and are therefore not siRNAs or shRNAs. Preferably, the antisense oligonucleotides of the present invention are single stranded. It is understood that single stranded oligonucleotides of the present invention can form hairpins or intermolecular duplex structures (duplex between two molecules of the same oligonucleotide), as long as the degree of intra or inter self-complementarity is less than 50% across of the full length of the oligonucleotide

Contiguous Nucleotide Sequence

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleotides of the oligonucleotide constitute the contiguous nucleotide sequence. In some embodiments the oligonucleotide comprises the contiguous nucleotide sequence, such as a F-G-F′ gapmer region, and may optionally comprise further nucleotide(s), for example a nucleotide linker region which may be used to attach a functional group to the contiguous nucleotide sequence. The nucleotide linker region may or may not be complementary to the target nucleic acid. Adventurously, the contiguous nucleotide sequence is 100% complementary to the target nucleic acid.

Nucleotides

Nucleotides are the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In a preferred embodiment the modified nucleoside comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

Modified Internucleoside Linkages

The term “modified internucleoside linkage” is defined as generally understood by the skilled person as linkages other than phosphodiester (PO) linkages, that covalently couples two nucleosides together. The oligonucleotides of the invention may therefore comprise modified internucleoside linkages. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use, and may serve to protect against nuclease cleavage at regions of DNA or RNA nucleosides in the oligonucleotide of the invention, for example within the gap region of a gapmer oligonucleotide, as well as in regions of modified nucleosides, such as region F and F′.

In an embodiment, the oligonucleotide comprises one or more internucleoside linkages modified from the natural phosphodiester, such one or more modified internucleoside linkages that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages. In some embodiments at least 50% of the internucleoside linkages in the oligonucleotide, or contiguous nucleotide sequence thereof, are modified, such as at least 60%, such as at least 70%, such as at least 80 or such as at least 90% of the internucleoside linkages in the oligonucleotide, or contiguous nucleotide sequence thereof, are nuclease resistant internucleoside linkages. In some embodiments all of the internucleoside linkages of the oligonucleotide, or contiguous nucleotide sequence thereof, are nuclease resistant internucleoside linkages. It will be recognized that, in some embodiments the nucleosides which link the oligonucleotide of the invention to a non-nucleotide functional group, such as a conjugate, may be phosphodiester.

A preferred modified internucleoside linkage is phosphorothioate.

Phosphorothioate internucleoside linkages are particularly useful due to nuclease resistance, beneficial pharmacokinetics and ease of manufacture. In some embodiments at least 50% of the internucleoside linkages in the oligonucleotide, or contiguous nucleotide sequence thereof, are phosphorothioate, such as at least 60%, such as at least 70%, such as at least 80% or such as at least 90% of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate.

Nuclease resistant linkages, such as phosphorothioate linkages, are particularly useful in oligonucleotide regions capable of recruiting nuclease when forming a duplex with the target nucleic acid, such as region G for gapmers. Phosphorothioate linkages may, however, also be useful in non-nuclease recruiting regions and/or affinity enhancing regions such as regions F and F′ for gapmers. Gapmer oligonucleotides may, in some embodiments comprise one or more phosphodiester linkages in region F or F′, or both region F and F′, which the internucleoside linkage in region G may be fully phosphorothioate.

Advantageously, all the internucleoside linkages in the contiguous nucleotide sequence of the oligonucleotide are phosphorothioate linkages.

It is recognized that, as disclosed in EP2 742 135, antisense oligonucleotide may comprise other internucleoside linkages (other than phosphodiester and phosphorothioate), for example alkyl phosphonate/methyl phosphonate internucleosides, which according to EP2 742 135 may for example be tolerated in an otherwise DNA phosphorothioate gap region.

Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In a some embodiments the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

Modified Oligonucleotide

The term modified oligonucleotide describes an oligonucleotide comprising one or more sugar-modified nucleosides and/or modified internucleoside linkages. The term chimeric” oligonucleotide is a term that has been used in the literature to describe oligonucleotides with modified nucleosides.

Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the number of nucleotides in percent of a contiguous nucleotide sequence in a nucleic acid molecule (e.g. oligonucleotide) which, at a given position, are complementary to (i.e. form Watson Crick base pairs with) a contiguous sequence of nucleotides, at a given position of a separate nucleic acid molecule (e.g. the target nucleic acid or target sequence). The percentage is calculated by counting the number of aligned bases that form pairs between the two sequences (when aligned with the target sequence 5′-3′ and the oligonucleotide sequence from 3′-5′), dividing by the total number of nucleotides in the oligonucleotide and multiplying by 100. In such a comparison a nucleobase/nucleotide which does not align (form a base pair) is termed a mismatch.

Preferably, insertions and deletions are not allowed in the calculation of % complementarity of a contiguous nucleotide sequence.

The term “fully complementary”, refers to 100% complementarity.

Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned bases that are identical (a match) between two sequences (e.g. in the contiguous nucleotide sequence of the compound of the invention and in the reference sequence), dividing that number by the total number of nucleotides in the aligned region and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RT ln(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

Target Nucleic Acid

According to the present invention, the target nucleic acid is a nucleic acid which encodes a mammalian CARD9 protein and may for example be a gene, a CARD9 RNA, a mRNA, a pre-mRNA, a mature mRNA or a cDNA sequence. The target may therefore be referred to as an CARD9 target nucleic acid.

In some embodiments, the target nucleic acid encodes a human CARD9 protein, such as the human CARD9 gene encoding pre-mRNA or mRNA sequences provided herein as SEQ ID NO 1, 2 or 9. Thus, the target nucleic acid may be selected from the group consisting of SEQ ID NO 1, SEQ ID NO 2 and SEQ ID NO 9.

In some embodiments, the target nucleic acid encodes a mouse CARD9 protein. Suitably, the target nucleic acid encoding a mouse CARD9 protein comprises a sequence as shown in SEQ ID NO: 5 or 6.

In some embodiments, the target nucleic acid encodes a porcine CARD9 protein. Suitably, the target nucleic acid encoding a porcine CARD9 protein comprises a sequence as shown in SEQ ID NO: 7 or 8.

In some embodiments, the target nucleic acid encodes a cynomolgus monkey CARD9 protein. Suitably, the target nucleic acid encoding a cynomolgus monkey CARD9 protein comprises a sequence as shown in SEQ ID NO: 3 or 4.

If employing the oligonucleotide of the invention in research or diagnostics the target nucleic acid may be a cDNA or a synthetic nucleic acid derived from DNA or RNA.

For in vivo or in vitro application, the oligonucleotide of the invention is typically capable of inhibiting the expression of the CARD9 target nucleic acid in a cell which is expressing the CARD9 target nucleic acid. The contiguous sequence of nucleobases of the oligonucleotide of the invention is typically complementary to the CARD9 target nucleic acid, as measured across the length of the oligonucleotide, optionally with the exception of one or two mismatches, and optionally excluding nucleotide based linker regions which may link the oligonucleotide to an optional functional group such as a conjugate, or other non-complementary terminal nucleotides (e.g. region D′ or D″). The target nucleic acid is a messenger RNA, such as a mature mRNA or a pre-mRNA which encodes mammalian CARD9 protein, such as human CARD9, e.g. the human CARD9 pre-mRNA sequence, such as that disclosed as SEQ ID NO 1, or CARD9 mature mRNA, such as that disclosed as SEQ ID NO 2 or 9. Further, the target nucleic acid may be a mouse CARD9 pre-mRNA sequence, such as that disclosed as SEQ ID NO 5, or mouse CARD9 mature mRNA, such as that disclosed as SEQ ID NO 6. Further, the target nucleic acid may be the porcine CARD9 pre-mRNA sequence, such as that disclosed as SEQ ID NO 7, or a porcine CARD9 mature mRNA, such as that disclosed as SEQ ID NO 8. Further, the target nucleic acid may be a cynomolgus monkey CARD9 pre-mRNA sequence, such as that disclosed as SEQ ID NO 3, or a cynomolgus monkey CARD9 mature mRNA, such as that disclosed as SEQ ID NO 4. SEQ ID NOs 1-9 are DNA sequences—it will be understood that target RNA sequences have uracil (U) bases in place of the thymidine bases (T).

| Target Nucleic Acid | Sequence ID |
|---|---|
| CARD9 Homo sapiens | SEQ ID NO 1 |
| pre-mRNA | |
| CARD9 Homo sapiens | SEQ ID NO 2 |
| mRNA, transcript variant 1 | |
| CARD9 Homo sapiens | SEQ ID NO 9 |
| mRNA, transcript variant 2 | |
| CARD9 Macaca fascicularis | SEQ ID NO 3 |
| pre-mRNA | |
| CARD9 Macaca fascicularis | SEQ ID NO 4 |
| mRNA | |
| CARD9 Mus musculus | SEQ ID NO 5 |
| pre-mRNA | |
| CARD9 Mus musculus | SEQ ID NO 6 |
| mRNA | |
| CARD9 Sus scrofa | SEQ ID NO 7 |
| pre-mRNA | |
| CARD9 Sus scrofa | SEQ ID NO 8 |
| mRNA | |

In some embodiments, the oligonucleotide of the invention targets SEQ ID NO 1.

In some embodiments, the oligonucleotide of the invention targets SEQ ID NO 2.

In some embodiments, the oligonucleotide of the invention targets SEQ ID NO 9.

In some embodiments, the oligonucleotide of the invention targets SEQ ID NO 3.

In some embodiments, the oligonucleotide of the invention targets SEQ ID NO 4.

In some embodiments, the oligonucleotide of the invention targets SEQ ID NO 5.

In some embodiments, the oligonucleotide of the invention targets SEQ ID NO 6.

In some embodiments, the oligonucleotide of the invention targets SEQ ID NO 7.

In some embodiments, the oligonucleotide of the invention targets SEQ ID NO 8.

In some embodiments, the oligonucleotide of the invention targets SEQ ID NO 1, 2 and 9.

In some embodiments, the oligonucleotide of the invention targets SEQ ID NO 1 and 2.

In some embodiments, the oligonucleotide of the invention targets SEQ ID NO 1 and 3.

In some embodiments, the oligonucleotide of the invention targets SEQ ID NO 1 and 5.

In some embodiments, the oligonucleotide of the invention targets SEQ ID NO 1 and 7.

In some embodiments, the oligonucleotide of the invention targets SEQ ID NO 1 and 9.

In some embodiments, the oligonucleotide of the invention targets SEQ ID NO 3 and 4.

In some embodiments, the oligonucleotide of the invention targets SEQ ID NO 5 and 6.

In some embodiments, the oligonucleotide of the invention targets SEQ ID NO 7 and 8.

Target Sequence

The term “target sequence” as used herein refers to a sequence of nucleotides present in the target nucleic acid which comprises the nucleobase sequence which is complementary to the oligonucleotide of the invention. In some embodiments, the target sequence consists of a region on the target nucleic acid which is complementary to the contiguous nucleotide sequence of the oligonucleotide of the invention.

Herein are provided numerous target sequence regions, as defined by regions of the human CARD9 pre-mRNA (using SEQ ID NO 1 as a reference) which may be targeted by the oligonucleotides of the invention.

In some embodiments the target sequence is longer than the complementary sequence of a single oligonucleotide, and may, for example represent a preferred region of the target nucleic acid which may be targeted by several oligonucleotides of the invention.

The oligonucleotide of the invention comprises a contiguous nucleotide sequence which is complementary to or hybridizes to the target nucleic acid, such as a sub-sequence of the target nucleic acid, such as a target sequence described herein.

The oligonucleotide comprises a contiguous nucleotide sequence which are complementary to a target sequence present in the target nucleic acid molecule. The contiguous nucleotide sequence (and therefore the target sequence) comprises of at least 10 contiguous nucleotides, such as 10, 11, 12, 13, 14, 15, 16, 17, 18, 19, 20, 21, 22, 23, 24, 25, 26, 27, 28, 29 or 30 contiguous nucleotides, such as from 12-25, such as from 14-18 contiguous nucleotides.

Target Sequence Regions

The inventors have identified effective sequences of the CARD9 target nucleic acid which may be targeted by the oligonucleotide of the invention.

The nucleic acid sequences of the target nucleic acids that may be targeted by the oligonucleotide of the invention are shown in the following table.

TABLE 1
Suitable target nucleic acids

| | | Start_ | End_on_ | |
|---|---|---|---|---|
| | Target | on_SEQ_ | SEQ_ID_ | |
| SEQ_ID | sequence - target nucleic acid | ID_NO_1 | NO_1 | length |
| 10 | CCCTTGTCTGTCAAAACTGTCCTG | 432 | 461 | 30 |
| | AATGGG | | | |
| 11 | GTCCCAACATGGGTAGTT | 701 | 718 | 18 |
| 12 | GGCCACTCTTGCATCATC | 960 | 977 | 18 |
| 13 | AACCTGCTCTCACCCAG | 1102 | 1118 | 17 |
| 14 | GTTCCTCTCTCAGACCCCATCTGT | 1260 | 1284 | 25 |
| | G | | | |
| 15 | AGGCCATGTCGGACTACGAGAAC | 1599 | 1623 | 25 |
| | GA | | | |
| 16 | TCGGTCATCGACCCCTC | 1670 | 1686 | 17 |
| 17 | AAGGTCCTGAACCCCGATGATGA | 1715 | 1738 | 24 |
| | G | | | |
| 18 | CCAACCTGGTCATCCGCAAACGG | 1758 | 1786 | 29 |
| | AAAGTG | | | |
| 19 | GTCCCCAGCCTAGTACCAAGACC | 2055 | 2078 | 24 |
| | C | | | |
| 20 | GTGCTCCTGGACATCCTGCAGCG | 2223 | 2254 | 32 |
| | GACCGGCCA | | | |
| 21 | GGGCTACGTGGCCTTCCTCGAGA | 2258 | 2320 | 63 |
| | GCCTGGAGCTCTACTACCCGCAG | | | |
| | CTGTACAAGAAGGTCAC | | | |
| 22 | CGCGTCTTCTCCATGATCATCGGT | 2337 | 2367 | 31 |
| | GAGTGAC | | | |
| 23 | CCATCCCTAGGAGCCCT | 2439 | 2455 | 17 |
| 24 | GATGACTTCATCAAGGAGCTG | 2635 | 2655 | 21 |
| 25 | GAGGAGAACTACGACCTGGCCAT | 2752 | 2777 | 26 |
| | GCG | | | |
| 26 | CGCTCATGCGGAACCGTGACC | 2810 | 2830 | 21 |
| 27 | CTCAAGCACAGCCTCAT | 2990 | 3006 | 17 |
| 28 | CATTGCCTTTTGCCCCCTTCAGGA | 3223 | 3247 | 25 |
| | G | | | |
| 29 | GAGCAGCCCCTACATCCAGGTAC | 3262 | 3284 | 23 |
| 30 | CAGGCCAACACCATCTTCTCCCTG | 3326 | 3367 | 42 |
| | CGCAAGGACCTCCGCCAG | | | |
| 31 | GCGAGGCCCGACGCCTCCGGGTA | 3369 | 3393 | 25 |
| | GG | | | |
| 32 | CGCCCACTCCGTGCCT | 3452 | 3467 | 16 |
| 33 | AGGAGATGTTCGAGCTG | 3820 | 3836 | 17 |
| 34 | AAGGACTCCAAGATGTACAAGGA | 3855 | 3895 | 41 |
| | CCGCATCGAGGCCATCCT | | | |
| 35 | ATCTTTGTTATTTGTTTTTG | 4025 | 4044 | 20 |
| 36 | TGATGAAGTCAATACTTCCC | 4209 | 4228 | 20 |
| 37 | AGGGAAAACCGTGTTCAACCTTCC | 4246 | 4269 | 24 |
| 38 | GCTCCACCTTACAGACTT | 4271 | 4288 | 18 |
| 39 | AAACATTCTGTCTTGTTTTACCAGT | 4375 | 4415 | 41 |
| | AGCTTTTTTTAATCTT | | | |
| 40 | CAAGTCACCATTGCGCT | 4919 | 4935 | 17 |
| 41 | TCCACATCAGGAGCCTTAAAACGA | 5122 | 5156 | 35 |
| | GACCCCTGGGG | | | |
| 42 | TCCAAAATTTATCAAATGTGCACG | 5563 | 5590 | 28 |
| | TGTG | | | |
| 43 | CACGCTGTGTCCACCG | 5604 | 5619 | 16 |
| 44 | CGGCTTGACGTCCTCCGG | 5747 | 5764 | 18 |
| 45 | TGGAGGATCCCGCTCTGTGCCCT | 6336 | 6358 | 23 |
| 46 | TTTTCTATGACCACAGAGCTCCG | 6409 | 6431 | 23 |
| 47 | CTGGCCTTCCTTCACCTGGGG | 6494 | 6514 | 21 |
| 48 | CCCAGCTCTCAGACAAAG | 6865 | 6882 | 18 |
| 49 | GCCCATCTTCAGCACAGGCAGCC | 6935 | 6968 | 34 |
| | CGTGCCGCAAT | | | |
| 50 | GGCTGGGGATAAGTAAAATGG | 7016 | 7036 | 21 |
| 51 | GAGAACAAACTACAGAGCCC | 7050 | 7069 | 20 |
| 52 | CTGTGTCCCGGTGCAGT | 7378 | 7394 | 17 |
| 53 | GGGGCTTCTAGCGGGC | 7413 | 7428 | 16 |
| 54 | GTGGTGATGAGGTAGGTGTTTGC | 7509 | 7531 | 23 |
| 55 | CAAGCCCCCATGTAGGC | 7729 | 7745 | 17 |
| 56 | CTGAAGGTTCTCTCCCAATTG | 8006 | 8026 | 21 |
| 57 | CATGCCCACAGATGCTTTGGAGT | 8104 | 8131 | 28 |
| | GATGG | | | |
| 58 | CAGAGTCTACACTGGACCCATGT | 8323 | 8345 | 23 |
| 59 | CAGGCACGACTCTCCTTTCCAGG | 8443 | 8465 | 23 |
| 60 | CCTCGGGCTTTGTTGTAGAAACAA | 8844 | 8872 | 29 |
| | TGGCC | | | |
| 61 | TGTGTCTTGGCATCTGAAATGCAG | 8910 | 8950 | 41 |
| | GCTACCCACACCGGCTC | | | |
| 62 | AGAACTACCGCAGGTAGGCG | 9187 | 9206 | 20 |
| 63 | CCCCAGGCTTCTCCAAAACGGGC | 9213 | 9240 | 28 |
| | TGGGG | | | |
| 64 | GCAGCGACAACACCGACAC | 9349 | 9367 | 19 |
| 65 | GAATCTGGTGCCCTGAAAG | 9501 | 9519 | 19 |
| 66 | GTTTGTTAAGCGGCACTCA | 9549 | 9567 | 19 |
| 67 | CATGCACACGCCATCTGTGTAAC | 9601 | 9623 | 23 |
| 68 | TTTCACCATGTAACACACAATACA | 9640 | 9668 | 29 |
| | TGCAT | | | |
| 69 | TAAATAAACAGCACGGGTG | 9701 | 9719 | 19 |

In some embodiments the target sequence is SEQ ID NO 10.

In some embodiments the target sequence is SEQ ID NO 11.

In some embodiments the target sequence is SEQ ID NO 12.

In some embodiments the target sequence is SEQ ID NO 13.

In some embodiments the target sequence is SEQ ID NO 14.

In some embodiments the target sequence is SEQ ID NO 15.

In some embodiments the target sequence is SEQ ID NO 16.

In some embodiments the target sequence is SEQ ID NO 17.

In some embodiments the target sequence is SEQ ID NO 18.

In some embodiments the target sequence is SEQ ID NO 19.

In some embodiments the target sequence is SEQ ID NO 20.

In some embodiments the target sequence is SEQ ID NO 21.

In some embodiments the target sequence is SEQ ID NO 22.

In some embodiments the target sequence is SEQ ID NO 23.

In some embodiments the target sequence is SEQ ID NO 24.

In some embodiments the target sequence is SEQ ID NO 25.

In some embodiments the target sequence is SEQ ID NO 26.

In some embodiments the target sequence is SEQ ID NO 27.

In some embodiments the target sequence is SEQ ID NO 28.

In some embodiments the target sequence is SEQ ID NO 29.

In some embodiments the target sequence is SEQ ID NO 30.

In some embodiments the target sequence is SEQ ID NO 31.

In some embodiments the target sequence is SEQ ID NO 32.

In some embodiments the target sequence is SEQ ID NO 33.

In some embodiments the target sequence is SEQ ID NO 34.

In some embodiments the target sequence is SEQ ID NO 35.

In some embodiments the target sequence is SEQ ID NO 36.

In some embodiments the target sequence is SEQ ID NO 37.

In some embodiments the target sequence is SEQ ID NO 38.

In some embodiments the target sequence is SEQ ID NO 39.

In some embodiments the target sequence is SEQ ID NO 40.

In some embodiments the target sequence is SEQ ID NO 41.

In some embodiments the target sequence is SEQ ID NO 42.

In some embodiments the target sequence is SEQ ID NO 43.

In some embodiments the target sequence is SEQ ID NO 44.

In some embodiments the target sequence is SEQ ID NO 45.

In some embodiments the target sequence is SEQ ID NO 46.

In some embodiments the target sequence is SEQ ID NO 47.

In some embodiments the target sequence is SEQ ID NO 48.

In some embodiments the target sequence is SEQ ID NO 49.

In some embodiments the target sequence is SEQ ID NO 50.

In some embodiments the target sequence is SEQ ID NO 51.

In some embodiments the target sequence is SEQ ID NO 52.

In some embodiments the target sequence is SEQ ID NO 53.

In some embodiments the target sequence is SEQ ID NO 54.

In some embodiments the target sequence is SEQ ID NO 55.

In some embodiments the target sequence is SEQ ID NO 56.

In some embodiments the target sequence is SEQ ID NO 57.

In some embodiments the target sequence is SEQ ID NO 58.

In some embodiments the target sequence is SEQ ID NO 59.

In some embodiments the target sequence is SEQ ID NO 60.

In some embodiments the target sequence is SEQ ID NO 61.

In some embodiments the target sequence is SEQ ID NO 62.

In some embodiments the target sequence is SEQ ID NO 63.

In some embodiments the target sequence is SEQ ID NO 64.

In some embodiments the target sequence is SEQ ID NO 65.

In some embodiments the target sequence is SEQ ID NO 66.

In some embodiments the target sequence is SEQ ID NO 67.

In some embodiments the target sequence is SEQ ID NO 68.

In some embodiments the target sequence is SEQ ID NO 69.

In a further aspect, the invention provides for an antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary to an exon region of SEQ ID NO 1, selected from the group consisting of Exon 1-Exon_13. The positions of Exons 1 to 13 (Ex_1 to Ex_13) are provided in the following table.

| Exon | start_SEQ ID NO 1 | end_SEQ ID NO 1 |
|---|---|---|
| Ex_1 | 1 | 150 |
| Ex_2 | 1588 | 1787 |
| Ex_3 | 2221 | 2358 |
| Ex_4 | 2537 | 2841 |
| Ex_5 | 2981 | 3160 |
| Ex_6 | 3245 | 3388 |
| Ex_7 | 3807 | 3932 |
| Ex_8 | 5854 | 6045 |
| Ex_9 | 6425 | 6466 |
| Ex_10 | 6837 | 6882 |
| Ex_11 | 8465 | 8541 |
| Ex_12 | 9123 | 9199 |
| Ex_13 | 9281 | 9726 |

In a further aspect, the invention provides for an antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary to an intron region of SEQ ID NO 1, selected from the group consisting of Intron_1-Intron_12. The positions of Intron 1 to 12 (Int_1 to Int 12) are provided in the following table.

| Intron | start_SEQ ID NO 1 | end_SEQ ID NO 1 |
|---|---|---|
| Int_1 | 151 | 1587 |
| Int_2 | 1788 | 2220 |
| Int_3 | 2359 | 2536 |
| Int_4 | 2842 | 2980 |
| Int_5 | 3161 | 3244 |
| Int_6 | 3389 | 3806 |
| Int_7 | 3933 | 5853 |
| Int_8 | 6046 | 6424 |
| Int_9 | 6467 | 6836 |
| Int_10 | 6883 | 8464 |
| Int_11 | 8542 | 9122 |
| Int_12 | 9200 | 9280 |

In a further aspect, the invention provides for an antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary to a region of SEQ ID NO 1, selected from the group consisting of 1-16; 22-48; 51-72; 74-86; 100-114; 123-165; 229-274; 314-328; 330-342; 344-360; 371-403; 432-471; 477-491; 495-507; 534-548; 576-595; 610-622; 636-664; 674-720; 756-775; 785-798; 800-814; 818-849; 851-865; 868-880; 896-937; 948-978; 990-1009; 1012-1042; 1056-1078; 1097-1130; 1132-1144; 1173-1186; 1195-1209; 1211-1233; 1259-1284; 1299-1311; 1335-1350; 1352-1366; 1384-1401; 1403-1422; 1424-1446; 1448-1473; 1485-1522; 1537-1556; 1580-1596; 1598-1623; 1628-1661; 1670-1686; 1700-1731; 1733-1752; 1764-1794; 1805-1828; 1841-1874; 1876-1910; 1918-1942; 1975-1994; 2009-2036; 2055-2078; 2110-2126; 2128-2152; 2154-2206; 2208-2221; 2230-2287; 2301-2320; 2322-2338; 2340-2371; 2396-2418; 2420-2432; 2435-2483; 2485-2506; 2528-2576; 2578-2633; 2635-2693; 2695-2732; 2734-2783; 2806-2849; 2890-2902; 2904-2924; 2936-2958; 2989-3012; 3014-3054; 3056-3073; 3075-3109; 3111-3169; 3204-3306; 3308-3402; 3441-3478; 3667-3695; 3697-3714; 3746-3773; 3775-3800; 3802-3847; 3858-3883; 3885-3913; 3924-3940; 3955-3969; 3971-3983; 3995-4013; 4019-4098; 4107-4133; 4138-4156; 4162-4178; 4192-4206; 4209-4228; 4244-4269; 4271-4288; 4312-4347; 4375-4415; 4454-4483; 4485-4525; 4588-4604; 4606-4618; 4644-4664; 4666-4684; 4718-4758; 4760-4801; 4810-4831; 4842-4860; 4877-4914; 4916-4936; 4938-4957; 4959-4980; 4991-5005; 5015-5038; 5053-5072; 5074-5087; 5118-5157; 5178-5190; 5205-5218; 5260-5275; 5278-5312; 5314-5326; 5345-5383; 5392-5436; 5485-5497; 5531-5546; 5563-5590; 5600-5632; 5634-5668; 5742-5764; 5791-5807; 5819-5839; 5866-5880; 5890-5915; 5917-5942; 5953-5979; 5981-6041; 6043-6061; 6063-6078; 6090-6102; 6144-6159; 6181-6199; 6227-6241; 6252-6279; 6286-6307; 6316-6389; 6391-6438; 6440-6456; 6458-6484; 6486-6532; 6540-6559; 6586-6611; 6627-6642; 6693-6729; 6765-6799; 6843-6874; 6932-6974; 6980-6995; 7015-7036; 7049-7071; 7094-7129; 7131-7144; 7151-7171; 7173-7207; 7209-7233; 7263-7276; 7323-7345; 7353-7410; 7413-7442; 7490-7502; 7508-7531; 7566-7578; 7580-7592; 7627-7654; 7656-7669; 7671-7688; 7705-7718; 7727-7772; 7774-7787; 7795-7823; 7838-7869; 7873-7903; 7915-7930; 7936-7958; 7960-7984; 7986-7998; 8005-8026; 8028-8045; 8066-8079; 8082-8136; 8138-8151; 8170-8183; 8211-8230; 8232-8263; 8265-8279; 8322-8362; 8381-8404; 8439-8465; 8492-8524; 8535-8552; 8635-8648; 8733-8745; 8768-8784; 8794-8807; 8811-8838; 8843-8872; 8910-8952; 8959-8976; 8983-9010; 9027-9042; 9044-9057; 9078-9102; 9111-9151; 9153-9175; 9186-9243; 9256-9272; 9278-9293; 9295-9310; 9312-9327; 9348-9361; 9363-9400; 9402-9429; 9438-9483; 9498-9521; 9549-9567; 9574-9592; 9594-9623; 9640-9668; and 9701-9726.

In a further aspect, the invention provides for an antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary to a region of SEQ ID NO 1, selected from the group consisting of 24-39; 100-113; 991-1003; 1223-1236; 1625-1639; 1718-1752; 1754-1776; 2020-2032; 2219-2248; 2250-2269; 2271-2299; 2337-2356; 2563-2576; 2578-2603; 2638-2655; 2674-2693; 2702-2717; 2740-2753; 2812-2837; 2889-2901; 2995-3018; 3020-3039; 3047-3078; 3083-3099; 3125-3145; 3284-3300; 3334-3348; 3353-3368; 3819-3847; 3862-3880; 3891-3914; 5953-5966; 6458-6473; 6829-6844; 6865-6888; 7263-7275; 7771-7783; 8537-8549; 9153-9175; 9186-9201; 9318-9331; 9348-9367; and 9369-9381.

In a further aspect, the invention provides for an antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary to a region of SEQ ID NO 1, selected from the group consisting of 1035-1052; 1364-1376; 1610-1623; 1625-1640; 1642-1656; 1709-1724; 1736-1752; 1762-1776; 1778-1794; 2223-2242; 2247-2305; 2307-2320; 2335-2348; 2563-2575; 2584-2602; 2642-2657; 2669-2693; 2697-2713; 2721-2734; 2741-2753; 2755-2772; 2807-2819; 2827-2845; 2989-3025; 3028-3055; 3057-3117; 3125-3140; 3143-3156; 3262-3282; 3284-3308; 3341-3360; 3811-3824; 3826-3847; 3855-3897; 3899-3917; 3921-3934; 5128-5144; 5168-5180; 5863-5882; 5893-5914; 6009-6032; 6040-6053; 6458-6472; 6852-6879; 7201-7213; 7996-8008; 8452-8465; 8915-8928; 8948-8960; 9117-9134; 9161-9175; 9186-9201; 9288-9305; and 9334-9367.

Target Cell

The term a “target cell” as used herein refers to a cell which is expressing the target nucleic acid. In some embodiments the target cell may be in vivo or in vitro. In some embodiments the target cell is a mammalian cell such as a rodent cell, such as a mouse cell or a rat cell, or a primate cell such as a monkey cell (e.g. a cynomolgus monkey cell) or a human cell, or a porcine cell.

In preferred embodiments the target cell expresses human CARD9 mRNA, such as the CARD9 pre-mRNA, e.g. SEQ ID NO 1, or CARD9 mature mRNA (e.g. SEQ ID NO 2 or 9). In some embodiments the target cell expresses monkey CARD9 mRNA, such as the CARD9 pre-mRNA, e.g. SEQ ID NO 3, or CARD9 mature mRNA (e.g. SEQ ID NO 4). In some embodiments the target cell expresses mouse CARD9 mRNA, such as the CARD9 pre-mRNA, e.g. SEQ ID NO 5, or CARD9 mature mRNA (e.g. SEQ ID NO 6). In some embodiments the target cell expresses porcine CARD9 mRNA, such as the CARD9 pre-mRNA, e.g. SEQ ID NO 6, or CARD9 mature mRNA (e.g. SEQ ID NO 7). The poly A tail of CARD9 mRNA is typically disregarded for antisense oligonucleotide targeting.

Naturally Occurring Variant

The term “naturally occurring variant” refers to variants of CARD9 gene or transcripts which originate from the same genetic loci as the target nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms (SNPs), and allelic variants. Based on the presence of the sufficient complementary sequence to the oligonucleotide, the oligonucleotide of the invention may therefore target the target nucleic acid and naturally occurring variants thereof.

The Homo sapiens CARD9 gene is located at chromosome 9, 136363956 . . . 136373681, complement (NC_000009.12, Gene ID 64170).

In some embodiments, the naturally occurring variants have at least 95% such as at least 98% or at least 99% homology to a mammalian CARD9 target nucleic acid, such as a target nucleic acid selected form the group consisting of SEQ ID NO 1, 2, 3, 4, 5, 6, 7, 8 and 9. In some embodiments the naturally occurring variants have at least 99% homology to the human CARD9 target nucleic acid of SEQ ID NO 1.

Modulation of Expression

The term “modulation of expression” as used herein is to be understood as an overall term for an oligonucleotide's ability to alter the amount of CARD9 protein or CARD9 mRNA when compared to the amount of CARD9 or CARD9 mRNA prior to administration of the oligonucleotide. Alternatively, modulation of expression may be determined by reference to a control experiment. It is generally understood that the control is an individual or target cell treated with a saline composition or an individual or target cell treated with a non-targeting oligonucleotide (mock).

One type of modulation is an oligonucleotide's ability to inhibit, down-regulate, reduce, suppress, remove, stop, block, prevent, lessen, lower, avoid or terminate expression of CARD9, e.g. by degradation of CARD9 mRNA.

High Affinity Modified Nucleosides

A high affinity modified nucleoside is a modified nucleotide which, when incorporated into the oligonucleotide enhances the affinity of the oligonucleotide for its complementary target, for example as measured by the melting temperature (T^{m}). A high affinity modified nucleoside of the present invention preferably result in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

Sugar Modifications

The oligomer of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′—OH group naturally found in DNA and RNA nucleosides. Substituents may, for example be introduced at the 2′, 3′, 4′ or 5′ positions.

2′ Sugar Modified Nucleosides.

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide.

Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

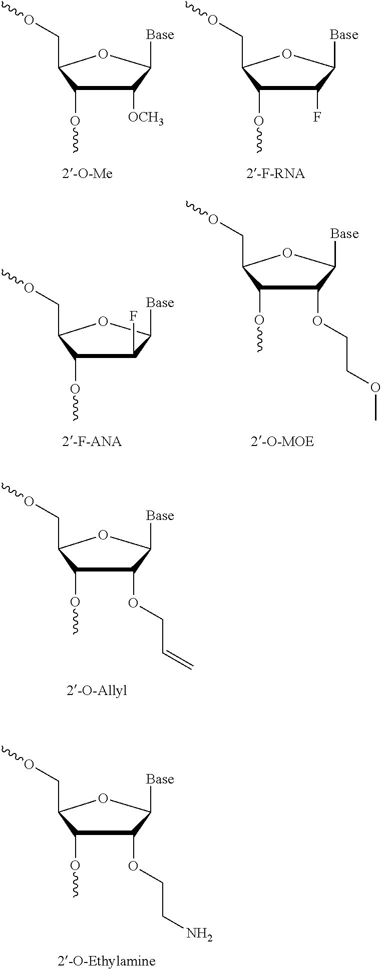

In relation to the present invention 2′ substituted does not include 2′ bridged molecules like LNA.

### Locked Nucleic Acids (LNA)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667.

Further non limiting, exemplary LNA nucleosides are disclosed in Scheme 1.

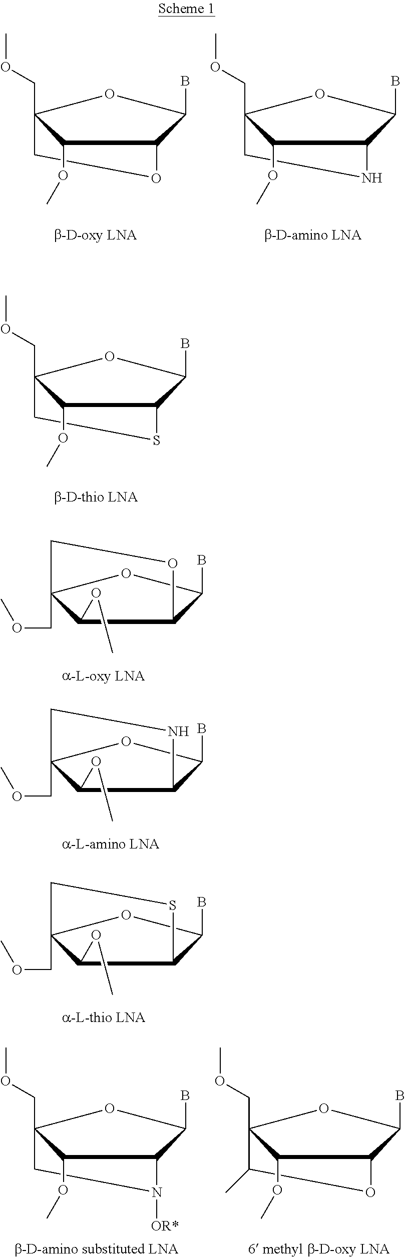

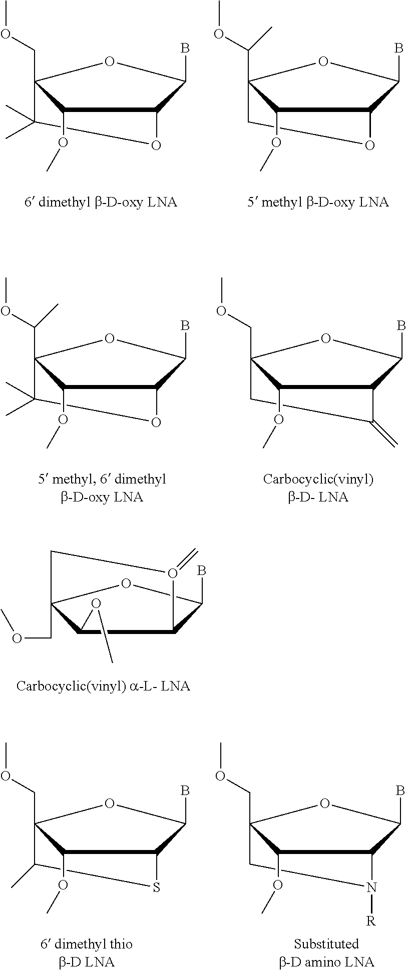

Particular LNA nucleosides are beta-D-oxy-LNA, 6′-methyl-beta-D-oxy LNA such as (S)-6′-methyl-beta-D-oxy-LNA (ScET) and ENA.

A particularly advantageous LNA is beta-D-oxy-LNA.

RNase H Activity and Recruitment

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/1/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO01/23613 (hereby incorporated by reference). For use in determining RHase H activity, recombinant human RNase H1 is available from Lubio Science GmbH, Lucerne, Switzerland.

Gapmer

The antisense oligonucleotide of the invention, or contiguous nucleotide sequence thereof may be a gapmer. The antisense gapmers are commonly used to inhibit a target nucleic acid via RNase H mediated degradation. A gapmer oligonucleotide comprises at least three distinct structural regions a 5′-flank, a gap and a 3′-flank, F-G-F′ in the ‘5->3’ orientation. The “gap” region (G) comprises a stretch of contiguous DNA nucleotides which enable the oligonucleotide to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the oligonucleotide for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

In a gapmer design, the 5′ and 3′ most nucleosides of the gap region are DNA nucleosides, and are positioned adjacent to a sugar modified nucleoside of the 5′ (F) or 3′ (F′) region respectively. The flanks may further defined by having at least one sugar modified nucleoside at the end most distant from the gap region, i.e. at the 5′ end of the 5′ flank and at the 3′ end of the 3′ flank.

Regions F-G-F′ form a contiguous nucleotide sequence. Antisense oligonucleotides of the invention, or the contiguous nucleotide sequence thereof, may comprise a gapmer region of formula F-G-F′.

The overall length of the gapmer design F-G-F′ may be, for example 12 to 32 nucleosides, such as 13 to 24, such as 14 to 22 nucleosides, Such as from 14 to 17, such as 16 to 18 nucleosides.

By way of example, the gapmer oligonucleotide of the present invention can be represented by the following formulae:

F₁₋₈-G₅₋₁₆-F′₁₋₈, such as

F₁₋₃-G₇₋₁₆-F′₂₋₃

with the proviso that the overall length of the gapmer regions F-G-F′ is at least 12, such as at least 14 nucleotides in length.

Regions F, G and F′ are further defined below and can be incorporated into the F-G-F′ formula.

Gapmer—Region G

Region G (gap region) of the gapmer is a region of nucleosides which enables the oligonucleotide to recruit RNaseH, such as human RNase H1, typically DNA nucleosides. RNaseH is a cellular enzyme which recognizes the duplex between DNA and RNA, and enzymatically cleaves the RNA molecule. Suitably gapmers may have a gap region (G) of at least 5 or 6 contiguous DNA nucleosides, such as 5-16 contiguous DNA nucleosides, such as 6-15 contiguous DNA nucleosides, such as 7-14 contiguous DNA nucleosides, such as 8-12 contiguous DNA nucleotides, such as 8-12 contiguous DNA nucleotides in length. The gap region G may, in some embodiments consist of 6, 7, 8, 9, 10, 11, 12, 13, 14, 15 or 16 contiguous DNA nucleosides. One or more cytosine (C) DNA in the gap region may in some instances be methylated (e.g. when a DNA c is followed by a DNA g) such residues are either annotated as 5-methyl-cytosine (meC) In some embodiments the gap region G may consist of 6, 7, 8, 9, 10, 11, 12, 13, 14, 15 or 16 contiguous phosphorothioate linked DNA nucleosides. In some embodiments, all internucleoside linkages in the gap are phosphorothioate linkages.

Whilst traditional gapmers have a DNA gap region, there are numerous examples of modified nucleosides which allow for RNaseH recruitment when they are used within the gap region. Modified nucleosides which have been reported as being capable of recruiting RNaseH when included within a gap region include, for example, alpha-L-LNA, C4′ alkylated DNA (as described in PCT/EP2009/050349 and Vester et al., Bioorg. Med. Chem. Lett. 18 (2008) 2296-2300, both incorporated herein by reference), arabinose derived nucleosides like ANA and 2F-ANA (Mangos et al. 2003 J. AM. CHEM. SOC. 125, 654-661), UNA (unlocked nucleic acid) (as described in Fluiter et al., Mol. Biosyst., 2009, 10, 1039 incorporated herein by reference). UNA is unlocked nucleic acid, typically where the bond between C2 and C3 of the ribose has been removed, forming an unlocked “sugar” residue. The modified nucleosides used in such gapmers may be nucleosides which adopt a 2′ endo (DNA like) structure when introduced into the gap region, i.e. modifications which allow for RNaseH recruitment). In some embodiments the DNA Gap region (G) described herein may optionally contain 1 to 3 sugar modified nucleosides which adopt a 2′ endo (DNA like) structure when introduced into the gap region.

Region G—“Gap-Breaker”

Alternatively, there are numerous reports of the insertion of a modified nucleoside which confers a 3′ endo conformation into the gap region of gapmers, whilst retaining some RNaseH activity. Such gapmers with a gap region comprising one or more 3′endo modified nucleosides are referred to as “gap-breaker” or “gap-disrupted” gapmers, see for example WO2013/022984. Gap-breaker oligonucleotides retain sufficient region of DNA nucleosides within the gap region to allow for RNaseH recruitment. The ability of gapbreaker oligonucleotide design to recruit RNaseH is typically sequence or even compound specific—see Rukov et al. 2015 Nucl. Acids Res. Vol. 43 pp. 8476-8487, which discloses “gapbreaker” oligonucleotides which recruit RNaseH which in some instances provide a more specific cleavage of the target RNA. Modified nucleosides used within the gap region of gap-breaker oligonucleotides may for example be modified nucleosides which confer a 3′endo confirmation, such 2′-O-methyl (OMe) or 2′-O-MOE (MOE) nucleosides, or beta-D LNA nucleosides (the bridge between C2′ and C4′ of the ribose sugar ring of a nucleoside is in the beta conformation), such as beta-D-oxy LNA or ScET nucleosides.

As with gapmers containing region G described above, the gap region of gap-breaker or gap-disrupted gapmers, have a DNA nucleosides at the 5′ end of the gap (adjacent to the 3′ nucleoside of region F), and a DNA nucleoside at the 3′ end of the gap (adjacent to the 5′ nucleoside of region F′). Gapmers which comprise a disrupted gap typically retain a region of at least 3 or 4 contiguous DNA nucleosides at either the 5′ end or 3′ end of the gap region.

Exemplary designs for gap-breaker oligonucleotides include

F₁₋₈-[D₃₋₄-E₁-D₃₋₄].F′₁₋₈

F₁₋₈-[D₁₋₄-E₁-D₃₋₄]-F′₁₋₈

F₁₋₈-[D₃₋₄-E₁-D₁₋₄]-F′₁₋₈

wherein region G is within the brackets [Dₙ-Eᵣ-Dₘ], D is a contiguous sequence of DNA nucleosides, E is a modified nucleoside (the gap-breaker or gap-disrupting nucleoside), and F and F′ are the flanking regions as defined herein, and with the proviso that the overall length of the gapmer regions F-G-F′ is at least 12, such as at least 14 nucleotides in length.

In some embodiments, region G of a gap disrupted gapmer comprises at least 6 DNA nucleosides, such as 6, 7, 8, 9, 10, 11, 12, 13, 14, 15 or 16 DNA nucleosides. As described above, the DNA nucleosides may be contiguous or may optionally be interspersed with one or more modified nucleosides, with the proviso that the gap region G is capable of mediating RNaseH recruitment.

Gapmer—Flanking Regions, F and F′

Region F is positioned immediately adjacent to the 5′ DNA nucleoside of region G. The 3′ most nucleoside of region F is a sugar modified nucleoside, such as a high affinity sugar modified nucleoside, for example a 2′ substituted nucleoside, such as a MOE nucleoside, or an LNA nucleoside.

Region F′ is positioned immediately adjacent to the 3′ DNA nucleoside of region G. The 5′ most nucleoside of region F′ is a sugar modified nucleoside, such as a high affinity sugar modified nucleoside, for example a 2′ substituted nucleoside, such as a MOE nucleoside, or an LNA nucleoside.

Region F is 1-8 contiguous nucleotides in length, such as 2-6, such as 3-4 contiguous nucleotides in length. Advantageously the 5′ most nucleoside of region F is a sugar modified nucleoside. In some embodiments the two 5′ most nucleoside of region F are sugar modified nucleoside. In some embodiments the 5′ most nucleoside of region F is an LNA nucleoside. In some embodiments the two 5′ most nucleoside of region F are LNA nucleosides. In some embodiments the two 5′ most nucleoside of region F are 2′ substituted nucleoside nucleosides, such as two 3′ MOE nucleosides. In some embodiments the 5′ most nucleoside of region F is a 2′ substituted nucleoside, such as a MOE nucleoside.

Region F′ is 2-8 contiguous nucleotides in length, such as 3-6, such as 4-5 contiguous nucleotides in length. Advantageously, embodiments the 3′ most nucleoside of region F′ is a sugar modified nucleoside. In some embodiments the two 3′ most nucleoside of region F′ are sugar modified nucleoside. In some embodiments the two 3′ most nucleoside of region F′ are LNA nucleosides. In some embodiments the 3′ most nucleoside of region F′ is an LNA nucleoside. In some embodiments the two 3′ most nucleoside of region F′ are 2′ substituted nucleoside nucleosides, such as two 3′ MOE nucleosides. In some embodiments the 3′ most nucleoside of region F′ is a 2′ substituted nucleoside, such as a MOE nucleoside. It should be noted that when the length of region F or F′ is one, it is advantageously an LNA nucleoside.

In some embodiments, region F and F′ independently consists of or comprises a contiguous sequence of sugar modified nucleosides. In some embodiments, the sugar modified nucleosides of region F may be independently selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In some embodiments, region F and F′ independently comprises both LNA and a 2′ substituted modified nucleosides (mixed wing design).

In some embodiments, region F and F′ consists of only one type of sugar modified nucleosides, such as only MOE or only beta-D-oxy LNA or only ScET. Such designs are also termed uniform flanks or uniform gapmer design.

In some embodiments, all the nucleosides of region F or F′, or F and F′ are LNA nucleosides, such as independently selected from beta-D-oxy LNA, ENA or ScET nucleosides.

In some embodiments, all the nucleosides of region F or F′, or F and F′ are 2′ substituted nucleosides, such as OMe or MOE nucleosides. In some embodiments region F consists of 1, 2, 3, 4, 5, 6, 7, or 8 contiguous OMe or MOE nucleosides. In some embodiments only one of the flanking regions can consist of 2′ substituted nucleosides, such as OMe or MOE nucleosides. In some embodiments it is the 5′ (F) flanking region that consists 2′ substituted nucleosides, such as OMe or MOE nucleosides whereas the 3′ (F′) flanking region comprises at least one LNA nucleoside, such as beta-D-oxy LNA nucleosides or cET nucleosides. In some embodiments it is the 3′ (F′) flanking region that consists 2′ substituted nucleosides, such as OMe or MOE nucleosides whereas the 5′ (F) flanking region comprises at least one LNA nucleoside, such as beta-D-oxy LNA nucleosides or cET nucleosides.

In some embodiments, all the modified nucleosides of region F and F′ are LNA nucleosides, such as independently selected from beta-D-oxy LNA, ENA or ScET nucleosides, wherein region F or F′, or F and F′ may optionally comprise DNA nucleosides (an alternating flank, see definition of these for more details). In some embodiments, all the modified nucleosides of region F and F′ are beta-D-oxy LNA nucleosides, wherein region F or F′, or F and F′ may optionally comprise DNA nucleosides (an alternating flank, see definition of these for more details).

In some embodiments the 5′ most and the 3′ most nucleosides of region F and F′ are LNA nucleosides, such as beta-D-oxy LNA nucleosides or ScET nucleosides.

In some embodiments, the internucleoside linkage between region F and region G is a phosphorothioate internucleoside linkage. In some embodiments, the internucleoside linkage between region F′ and region G is a phosphorothioate internucleoside linkage. In some embodiments, the internucleoside linkages between the nucleosides of region F or F′, F and F′ are phosphorothioate internucleoside linkages.

LNA Gapmer

An LNA gapmer is a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In some embodiments the LNA gapmer is of formula: [LNA]₁₋₅-[region G]-[LNA]₁₋₅, wherein region G is as defined in the Gapmer region G definition.

MOE Gapmers

A MOE gapmers is a gapmer wherein regions F and F′ consist of MOE nucleosides. In some embodiments the MOE gapmer is of design [MOE]₁₋₈-[Region G]-[MOE]₁₋₈, such as [MOE]₂₋₇-[Region G]₅₋₁₆-[MOE]₂₋₇, such as [MOE]₃₋₆-[Region G]-[MOE]₃₋₆, wherein region G is as defined in the Gapmer definition. MOE gapmers with a 5-10-5 design (MOE-DNA-MOE) have been widely used in the art.

Mixed Wing Gapmer

A mixed wing gapmer is an LNA gapmer wherein one or both of region F and F′ comprise a 2′ substituted nucleoside, such as a 2′ substituted nucleoside independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units, such as a MOE nucleosides. In some embodiments wherein at least one of region F and F′, or both region F and F′ comprise at least one LNA nucleoside, the remaining nucleosides of region F and F′ are independently selected from the group consisting of MOE and LNA. In some embodiments wherein at least one of region F and F′, or both region F and F′ comprise at least two LNA nucleosides, the remaining nucleosides of region F and F′ are independently selected from the group consisting of MOE and LNA. In some mixed wing embodiments, one or both of region F and F′ may further comprise one or more DNA nucleosides.

Mixed wing gapmer designs are disclosed in WO2008/049085 and WO2012/109395, both of which are hereby incorporated by reference.

Alternating Flank Gapmers

Oligonucleotides with alternating flanks are LNA gapmer oligonucleotides where at least one of the flanks (F or F′) comprises DNA in addition to the LNA nucleoside(s). In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F and/or F′ region are LNA nucleosides.

In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F or F′ region are LNA nucleosides, and there is at least one DNA nucleoside positioned between the 5′ and 3′ most LNA nucleosides of region F or F′ (or both region F and F′).

Region D′ or D″ in an Oligonucleotide

The oligonucleotide of the invention may in some embodiments comprise or consist of the contiguous nucleotide sequence of the oligonucleotide which is complementary to the target nucleic acid, such as the gapmer F-G-F′, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein. The addition of region D′ or D″ may be used for the purpose of joining the contiguous nucleotide sequence, such as the gapmer, to a conjugate moiety or another functional group. When used for joining the contiguous nucleotide sequence with a conjugate moiety is can serve as a biocleavable linker. Alternatively, it may be used to provide exonuclease protection or for ease of synthesis or manufacture.

Region D′ and D″ can be attached to the 5′ end of region F or the 3′ end of region F′, respectively to generate designs of the following formulas D′-F-G-F′, F-G-F′-D″ or D′-F-G-F′-D″. In this instance the F-G-F′ is the gapmer portion of the oligonucleotide and region D′ or D″ constitute a separate part of the oligonucleotide.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs (e.g. gapmer regions) within a single oligonucleotide.

In one embodiment the oligonucleotide of the invention comprises a region D′ and/or D″ in addition to the contiguous nucleotide sequence which constitutes the gapmer.

In some embodiments, the oligonucleotide of the present invention can be represented by the following formulae:

F-G-F′; in particular F₁₋₈-G₅₋₁₆-F′₂₋₈

D′-F-G-F′, in particular D′₁₋₃-F₁₋₈-G₅₋₁₆-F′₂₋₈

F-G-F′-D″, in particular F₁₋₈-G₅₋₁₆-F′₂₋₈-D″₁₋₃

D′-F-G-F′-D″, in particular D′₁₋₃-F₁₋₈-G₅₋₁₆-F′₂₋₈-D″₁₋₃

In some embodiments the internucleoside linkage positioned between region D′ and region F is a phosphodiester linkage. In some embodiments the internucleoside linkage positioned between region F′ and region D″ is a phosphodiester linkage.

Conjugate

The term conjugate as used herein refers to an oligonucleotide which is covalently linked to a non-nucleotide moiety (conjugate moiety or region C or third region).

Conjugation of the oligonucleotide of the invention to one or more non-nucleotide moieties may improve the pharmacology of the oligonucleotide, e.g. by affecting the activity, cellular distribution, cellular uptake or stability of the oligonucleotide. In some embodiments the conjugate moiety modify or enhance the pharmacokinetic properties of the oligonucleotide by improving cellular distribution, bioavailability, metabolism, excretion, permeability, and/or cellular uptake of the oligonucleotide. In particular the conjugate may target the oligonucleotide to a specific organ, tissue or cell type and thereby enhance the effectiveness of the oligonucleotide in that organ, tissue or cell type. At the same time the conjugate may serve to reduce activity of the oligonucleotide in non-target cell types, tissues or organs, e.g. off target activity or activity in non-target cell types, tissues or organs.

In an embodiment, the non-nucleotide moiety (conjugate moiety) is selected from the group consisting of carbohydrates, cell surface receptor ligands, drug substances, hormones, lipophilic substances, polymers, proteins, peptides, toxins (e.g. bacterial toxins), vitamins, viral proteins (e.g. capsids) or combinations thereof.

Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety (Region C), to a first region, e.g. an oligonucleotide or contiguous nucleotide sequence or gapmer region F-G-F′ (region A).

In some embodiments of the invention the conjugate or oligonucleotide conjugate of the invention may optionally, comprise a linker region (second region or region B and/or region Y) which is positioned between the oligonucleotide or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. DNA phosphodiester containing biocleavable linkers are described in more detail in WO 2014/076195 (hereby incorporated by reference)—see also region D′ or D″ herein.

Region Y refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups. The oligonucleotide conjugates of the present invention can be constructed of the following regional elements A-C, A-B-C, A-B-Y-C, A-Y-B-C or A-Y-C. In some embodiments the linker (region Y) is an amino alkyl, such as a C2-C36 amino alkyl group, including, for example C6 to C12 amino alkyl groups. In a preferred embodiment the linker (region Y) is a C6 amino alkyl group.

Treatment

The term ‘treatment’ as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic.

### DETAILED DESCRIPTION OF THE INVENTION

The invention relates to oligonucleotides, such as antisense oligonucleotides, targeting CARD9 expression.

The oligonucleotides of the invention targeting CARD9 are capable of hybridizing to and inhibiting the expression of a CARD9 target nucleic acid in a cell which is expressing the CARD9 target nucleic acid.

The CARD9 target nucleic acid may be a mammalian CARD9 mRNA or premRNA, such as a human, mouse, porcine or monkey CARD9 mRNA or premRNA. In some embodiments, the CARD9 target nucleic acid is CARD9 mRNA or premRNA for example a premRNA or mRNA originating from the Homo sapiens (CARD9), RefSeqGene on chromosome 9, exemplified by NCBI Reference Sequence NG_021197.1 (SEQ ID NO 1).

The human CARD9 pre-mRNA is encoded on Homo sapiens Chromosome 9, NC_000009.12 (136363956 . . . 136373681, complement). GENE ID=64170 (CARD9).

Mature human mRNA target sequence is illustrated herein by the cDNA sequences SEQ ID NO 2 and 9. A mature monkey mRNA target sequence is illustrated herein by the cDNA sequence shown in SEQ ID NO 4. A mature mouse mRNA target sequence is illustrated herein by the cDNA sequence shown in SEQ ID NO 6. A mature porcine mRNA target sequence is illustrated herein by the cDNA sequence shown in SEQ ID NO 8.

The oligonucleotides of the invention are capable of inhibiting the expression of CARD9 target nucleic acid, such as the CARD9 mRNA, in a cell which is expressing the target nucleic acid, such as the CARD9 mRNA (e.g. a human, monkey, mouse or pig cell).

In some embodiments, the oligonucleotides of the invention are capable of inhibiting the expression of CARD9 target nucleic acid in a cell which is expressing the target nucleic acid, so to reduce the level of CARD9 target nucleic acid (e.g. the mRNA) by at least 50%, at least 60%, at least 70%, at least 80%, or at least 90% inhibition compared to the expression level of the CARD9 target nucleic acid (e.g. the mRNA) in the cell. Suitably the cell is selected from the group consisting of a human cell, a monkey cell, a mouse cell and pig cell. In some embodiments, the cell is human cell such a THP-1 cell. THP-1 is a human monocytic cell line derived from an acute monocytic leukemia patient. Example 1 provides a suitable assay for evaluating the ability of the oligonucleotides of the invention to inhibit the expression of the target nucleic acid. Suitably the evaluation of a compounds ability to inhibit the expression of the target nucleic acid is performed in vitro, such a gymnotic in vitro assay, for example as according to Example 1.

An aspect of the present invention relates to an antisense oligonucleotide, such as an LNA antisense oligonucleotide gapmer which comprises a contiguous nucleotide sequence of 10 to 30 nucleotides in length with at least 90% complementarity, such as is fully complementary to SEQ ID NO 1. 2, 3, 4, 5, 6, 7, 8 or 9 (e.g. SEQ ID NO 1, 2 and 9).

In some embodiments, the oligonucleotide comprises a contiguous sequence of 10-30 nucleotides, which is at least 90% complementary, such as at least 91%, such as at least 92%, such as at least 93%, such as at least 94%, such as at least 95%, such as at least 96%, such as at least 97%, such as at least 98%, or 100% complementary with a region of the target nucleic acid or a target sequence. The sequences of suitable target nucleic acids are described herein above (see Table 1).

In some embodiments, the oligonucleotide of the invention comprises a contiguous nucleotides sequence of 12-24, such as 13, 14, 15, 16, 17, 18, 19, 20, 21, 22, or 23, contiguous nucleotides in length, wherein the contiguous nucleotide sequence is fully complementary to a target nucleic acid provided in Table 1 above (i.e. to SEQ ID NO 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68 or 69).

In some embodiments, the antisense oligonucleotide of the invention comprises a contiguous nucleotides sequence of 12-15, such as 13, or 14, 15 contiguous nucleotides in length, wherein the contiguous nucleotide sequence is fully complementary to a target nucleic acid provided in Table 1 above.

Typically, the antisense oligonucleotide of the invention or the contiguous nucleotide sequence thereof is a gapmer, such as an LNA gapmer, a mixed wing gapmer, or an alternating flank gapmer.

In some embodiments, the antisense oligonucleotide according to the invention, comprises a contiguous nucleotide sequence of at least 10 contiguous nucleotides, such as at least 12 contiguous nucleotides, such as at least 13 contiguous nucleotides, such as at least 14 contiguous nucleotides, such as at least 15 contiguous nucleotides, which is fully complementary to SEQ NO 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68 or 69.

In some embodiments the contiguous nucleotide sequence of the antisense oligonucleotide according to the invention is less than 20 nucleotides in length. In some embodiments the contiguous nucleotide sequence of the antisense oligonucleotide according to the invention is 12-24 nucleotides in length. In some embodiments the contiguous nucleotide sequence of the antisense oligonucleotide according to the invention is 12-22 nucleotides in length. In some embodiments the contiguous nucleotide sequence of the antisense oligonucleotide according to the invention is 12-20 nucleotides in length. In some embodiments the contiguous nucleotide sequence of the antisense oligonucleotide according to the invention is 12-18 nucleotides in length. In some embodiments the contiguous nucleotide sequence of the antisense oligonucleotide according to the invention is 12-16 nucleotides in length. Advantageously, in some embodiments all of the internucleoside linkages between the nucleosides of the contiguous nucleotide sequence are phosphorothioate internucleoside linkages.

In some embodiments, the contiguous nucleotide sequence is fully complementary to SEQ NO 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68 or 69.

In some embodiments, the antisense oligonucleotide is a gapmer oligonucleotide comprising a contiguous nucleotide sequence of formula 5′-F-G-F′-3′, where region F and F′ independently comprise 1-8 sugar modified nucleosides, and G is a region between 5 and 16 nucleosides which are capable of recruiting RNaseH.

In some embodiments, the sugar modified nucleosides of region F and F′ are independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.

In some embodiments, region G comprises 5-16 contiguous DNA nucleosides.

In some embodiments, wherein the antisense oligonucleotide is a gapmer oligonucleotide, such as an LNA gapmer oligonucleotide.

In some embodiments, the LNA nucleosides are beta-D-oxy LNA nucleosides.

In some embodiments, the internucleoside linkages between the contiguous nucleotide sequence are phosphorothioate internucleoside linkages.

Preferred sequences motifs and antisense oligonucleotides of the present invention are shown in Table 2.

TABLE 2
Sequence Motifs and Compounds of the Invention

| | | | | Compound |
|---|---|---|---|---|
| SEQ | | Compound | Oligonucleotide | LNA |
| ID NO | Sequence motif | ID | compound | pattern |
| 70 | AGGACAGTTTTGACAGACA | 70_1 | AggacagttttgacagaCA | 1-16-2 |
| 71 | TCAGGACAGTTTTGACAGA | 71_1 | TcaggacagttttgacaGA | 1-16-2 |
| 72 | ATTCAGGACAGTTTTGACA | 72_1 | AttcaggacagttttgacAG | 1-17-2 |
| | G | | | |
| 73 | ATTCAGGACAGTTTTGACA | 73_1 | AttcaggacagttttgACA | 1-15-3 |
| 74 | ATTCAGGACAGTTTTGAC | 74_1 | ATtcaggacagttttGAC | 2-13-3 |
| 75 | CATTCAGGACAGTTTTGAC | 75_1 | CATtcaggacagttttgAC | 3-14-2 |
| 76 | CCATTCAGGACAGTTTTGA | 76_1 | CcattcaggacagttttgAC | 1-17-2 |
| | C | | | |
| 77 | CATTCAGGACAGTTTTGA | 77_1 | CATtcaggacagttttGA | 3-13-2 |
| 77 | CATTCAGGACAGTTTTGA | 77_2 | CAttcaggacagttttGA | 2-14-2 |
| 78 | CCATTCAGGACAGTTTTGA | 78_1 | CcattcaggacagttttGA | 1-16-2 |
| 79 | CCATTCAGGACAGTTTTG | 79_1 | CcattcaggacagtttTG | 1-15-2 |
| 80 | CCCATTCAGGACAGTTTTG | 80_1 | CccattcaggacagtttTG | 1-16-2 |
| 81 | CCCATTCAGGACAGTTTT | 81_1 | CccattcaggacagttTT | 1-15-2 |
| 82 | CCCATTCAGGACAGTTT | 82_1 | CccattcaggacagtTT | 1-14-2 |
| 83 | CTACCCATGTTGGGAC | 83_1 | CtacccatgttgggAC | 1-13-2 |
| 84 | ACTACCCATGTTGGGAC | 84_1 | ActacccatgttgggAC | 1-14-2 |
| 85 | AACTACCCATGTTGGGAC | 85_1 | AactacccatgttgggAC | 1-15-2 |
| 86 | AACTACCCATGTTGGGA | 86_1 | AactacccatgttggGA | 1-14-2 |
| 87 | ATGATGCAAGAGTGGCC | 87_1 | AtgatgcaagagtggCC | 1-14-2 |
| 88 | CTGGGTGAGAGCAGGTT | 88_1 | CtgggtgagagcaggTT | 1-14-2 |
| 89 | GGGGTCTGAGAGAGGAAC | 89_1 | GgggtctgagagaggAAC | 1-14-3 |
| 90 | TGGGGTCTGAGAGAGGAA | 90_1 | TGgggtctgagagaggAA | 2-14-2 |
| 90 | TGGGGTCTGAGAGAGGAA | 90_2 | TggggtctgagagaggAA | 1-15-2 |
| 91 | ATGGGGTCTGAGAGAGGA | 91 1 | ATggggtctgagagaggAA | 2-15-2 |
| | A | | | |
| 91 | ATGGGGTCTGAGAGAGGA | 91 2 | AtggggtctgagagaggAA | 1-16-2 |
| | A | | | |
| 92 | GATGGGGTCTGAGAGAGG | 92_1 | GAtggggtctgagagaggAA | 2-16-2 |
| | AA | | | |
| 93 | ATGGGGTCTGAGAGAGGA | 93_1 | AtggggtctgagagagGA | 1-15-2 |
| 94 | AGATGGGGTCTGAGAGAG | 94_1 | AgatggggtctgagagaGG | 1-16-2 |
| | G | | | |
| 95 | GATGGGGTCTGAGAGAG | 95_1 | GATggggtctgagagAG | 3-12-2 |
| 95 | GATGGGGTCTGAGAGAG | 95_2 | GatggggtctgagaGAG | 1-13-3 |
| 96 | AGATGGGGTCTGAGAGAG | 96_1 | AgatggggtctgagaGAG | 1-14-3 |
| 96 | AGATGGGGTCTGAGAGAG | 96_2 | AgatggggtctgagagAG | 1-15-2 |
| 97 | CAGATGGGGTCTGAGAGA | 97_1 | CagatggggtctgagagAG | 1-16-2 |
| | G | | | |
| 98 | ACAGATGGGGTCTGAGAG | 98_1 | AcagatggggtctgagagAG | 1-17-2 |
| | AG | | | |
| 99 | CAGATGGGGTCTGAGAGA | 99_1 | CagatggggtctgagAGA | 1-14-3 |
| 99 | CAGATGGGGTCTGAGAGA | 99_2 | CagatggggtctgagaGA | 1-15-2 |
| 100 | ACAGATGGGGTCTGAGAG | 100_1 | AcagatggggtctgaGAG | 1-14-3 |
| 100 | ACAGATGGGGTCTGAGAG | 100_2 | ACagatggggtctgagAG | 2-14-2 |
| 100 | ACAGATGGGGTCTGAGAG | 100_3 | AcagatggggtctgagAG | 1-15-2 |
| 101 | CACAGATGGGGTCTGAGA | 101_1 | CacagatggggtctgagAG | 1-16-2 |
| | G | | | |
| 102 | TAGTCCGACATGGCCT | 102_1 | Tagtc^{m}cgacatggcCT | 1-13-2 |
| 103 | TAGTCCGACATGGCC | 103_1 | Tagtc^{m}cgacatggCC | 1-12-2 |
| 104 | TTCTCGTAGTCCGACATG | 104_1 | Ttct^{m}cgtagtc^{m}cgacATG | 1-14-3 |
| 104 | TTCTCGTAGTCCGACATG | 104_2 | Ttct^{m}cgtagtc^{m}cgacaTG | 1-15-2 |
| 105 | GTTCTCGTAGTCCGACATG | 105_1 | Gttct^{m}cgtagtc^{m}cgacaTG | 1-16-2 |
| 106 | TCTCGTAGTCCGACAT | 106_1 | Tct^{m}cgtagtc^{m}cgaCAT | 1-12-3 |
| 107 | TTCTCGTAGTCCGACAT | 107_1 | TTct^{m}cgtagtc^{m}cgaCAT | 2-12-3 |
| 107 | TTCTCGTAGTCCGACAT | 107_2 | Ttct^{m}cgtagtc^{m}cgaCAT | 1-13-3 |
| 108 | GTTCTCGTAGTCCGACAT | 108_1 | Gttct^{m}cgtagtc^{m}cgaCAT | 1-14-3 |
| 108 | GTTCTCGTAGTCCGACAT | 108_2 | Gttct^{m}cgtagtc^{m}cgacAT | 1-15-2 |
| 109 | CGTTCTCGTAGTCCGACAT | 109_1 | Cgttct^{m}cgtagtc^{m}cgacAT | 1-16-2 |
| 110 | GTTCTCGTAGTCCGACA | 110_1 | Gttct^{m}cgtagtc^{m}cgaCA | 1-14-2 |
| 111 | CGTTCTCGTAGTCCGACA | 111_1 | Cgttct^{m}cgtagtc^{m}cgaCA | 1-15-2 |
| 112 | CGTTCTCGTAGTCCGA | 112_1 | Cgttct^{m}cgtagtccGA | 1-13-2 |
| 113 | GGGGTCGATGACCGA | 113_1 | Ggggt^{m}cgatgaccGA | 1-12-2 |
| 114 | AGGGGTCGATGACCGA | 114_1 | Aggggt^{m}cgatgaccGA | 1-13-2 |
| 115 | AGGGGTCGATGACCG | 115_1 | Aggggt^{m}cgatgACCG | 1-10-4 |
| 115 | AGGGGTCGATGACCG | 115_2 | AGgggt^{m}cgatgacCG | 2-11-2 |
| 116 | GAGGGGTCGATGACCG | 116_1 | Gaggggt^{m}cgatgaCCG | 1-12-3 |
| 117 | TCGGGGTTCAGGACCTT | 117_1 | T^{m}cggggttcaggaccTT | 1-14-2 |
| 118 | CATCGGGGTTCAGGAC | 118_1 | CAt^{m}cggggttcaggAC | 2-12-2 |
| 119 | TCATCGGGGTTCAGGAC | 119_1 | TCat^{m}cggggttcaggAC | 2-13-2 |
| 119 | TCATCGGGGTTCAGGAC | 119_2 | Tcat^{m}cggggttcaggAC | 1-14-2 |
| 120 | ATCATCGGGGTTCAGGAC | 120_1 | Atcat^{m}cggggttcagGAC | 1-14-3 |
| 120 | ATCATCGGGGTTCAGGAC | 120_2 | Atcat^{m}cggggttcaggAC | 1-15-2 |
| 121 | CATCATCGGGGTTCAGGA | 121_1 | Catcat^{m}cggggttcaggAC | 1-16-2 |
| | C | | | |
| 122 | ATCATCGGGGTTCAGGA | 122_1 | Atcat^{m}cggggttcaGGA | 1-13-3 |
| 122 | ATCATCGGGGTTCAGGA | 122_2 | Atcat^{m}cggggttcagGA | 1-14-2 |
| 123 | CATCATCGGGGTTCAGGA | 123_1 | Catcat^{m}cggggttcagGA | 1-15-2 |
| 124 | ATCATCGGGGTTCAGG | 124_1 | Atcat^{m}cggggttcaGG | 1-13-2 |
| 125 | CATCATCGGGGTTCAGG | 125_1 | Catcat^{m}cggggttcaGG | 1-14-2 |
| 126 | CATCATCGGGGTTCAG | 126_1 | CAtcat^{m}cggggttCAG | 2-11-3 |
| 126 | CATCATCGGGGTTCAG | 126_2 | CAtcat^{m}cggggttcAG | 2-12-2 |
| 127 | TCATCATCGGGGTTCAG | 127_1 | TCAtcat^{m}cggggttcAG | 3-12-2 |
| 127 | TCATCATCGGGGTTCAG | 127_2 | TCatcat^{m}cggggttcAG | 2-13-2 |
| 128 | CTCATCATCGGGGTTCAG | 128_1 | Ctcatcat^{m}cggggttcAG | 1-15-2 |
| 129 | TCATCATCGGGGTTCA | 129_1 | TCAtcat^{m}cggggttCA | 3-11-2 |
| 129 | TCATCATCGGGGTTCA | 129_2 | TCatcat^{m}cggggttCA | 2-12-2 |
| 130 | CGGATGACCAGGTTGG | 130_1 | CggatgaccaggtTGG | 1-12-3 |
| 131 | GCGGATGACCAGGTTG | 131_1 | G^{m}cggatgaccaggTTG | 1-12-3 |
| 131 | GCGGATGACCAGGTTG | 131_2 | G^{m}cggatgaccaggtTG | 1-13-2 |
| 132 | TGCGGATGACCAGGTTG | 132_1 | Tg^{m}cggatgaccaggtTG | 1-14-2 |
| 133 | TTGCGGATGACCAGGTTG | 133_1 | TTg^{m}cggatgaccaggtTG | 2-14-2 |
| 133 | TTGCGGATGACCAGGTTG | 133_2 | Ttg^{m}cggatgaccaggtTG | 1-15-2 |
| 134 | TGCGGATGACCAGGTT | 134_1 | TG^{m}cggatgaccagGTT | 2-11-3 |
| 134 | TGCGGATGACCAGGTT | 134_2 | TG^{m}cggatgaccaggTT | 2-12-2 |
| 135 | TTGCGGATGACCAGGTT | 135_1 | TTG^{m}cggatgaccaggTT | 3-12-2 |
| 136 | TTGCGGATGACCAGGT | 136_1 | TTG^{m}cggatgaccagGT | 3-11-2 |
| 137 | CGTTTGCGGATGACCA | 137_1 | Cgtttg^{m}cggatgaCCA | 1-12-3 |
| 137 | CGTTTGCGGATGACCA | 137_2 | Cgtttg^{m}cggatgacCA | 1-13-2 |
| 138 | CCGTTTGCGGATGACCA | 138_1 | C^{m}cgtttg^{m}cggatgacCA | 1-14-2 |
| 139 | CGTTTGCGGATGACC | 139_1 | Cgtttg^{m}cggatgACC | 1-11-3 |
| 140 | CCGTTTGCGGATGACC | 140_1 | C^{m}cgtttg^{m}cggatgaCC | 1-13-2 |
| 141 | TTCCGTTTGCGGATGA | 141_1 | Ttc^{m}cgtttg^{m}cggaTGA | 1-12-3 |
| 142 | TTTCCGTTTGCGGATGA | 142_1 | TTtc^{m}cgtttg^{m}cggaTGA | 2-12-3 |
| 142 | TTTCCGTTTGCGGATGA | 142_2 | Tttc^{m}cgtttg^{m}cggatGA | 1-14-2 |
| 143 | CTTTCCGTTTGCGGATGA | 143_1 | Ctttc^{m}cgtttg^{m}cggatGA | 1-15-2 |
| 144 | TTCCGTTTGCGGATG | 144_1 | TTCCgtttg^{m}cggaTG | 4-9-2 |
| 144 | TTCCGTTTGCGGATG | 144_2 | TTC^{m}cgtttg^{m}cggaTG | 3-10-2 |
| 145 | CTTTCCGTTTGCGGATG | 145_1 | Ctttc^{m}cgtttg^{m}cggaTG | 1-14-2 |
| 146 | ACTTTCCGTTTGCGGATG | 146_1 | ACtttc^{m}cgtttg^{m}cggaTG | 2-14-2 |
| 147 | CTTTCCGTTTGCGGAT | 147_1 | CTttc^{m}cgtttg^{m}cggAT | 2-12-2 |
| 148 | ACTTTCCGTTTGCGGAT | 148_1 | Actttc^{m}cgtttg^{m}cggAT | 1-14-2 |
| 149 | GGTACTAGGCTGGGGAC | 149_1 | GgtactaggctggggAC | 1-14-2 |
| 150 | TGGTACTAGGCTGGGGA | 150_1 | TggtactaggctgggGA | 1-14-2 |
| 151 | TTGGTACTAGGCTGGGGA | 151_1 | TtggtactaggctgggGA | 1-15-2 |
| 152 | TTGGTACTAGGCTGGGG | 152_1 | TTggtactaggctggGG | 2-13-2 |
| 153 | TCTTGGTACTAGGCTGGG | 153_1 | TcttggtactaggctgGG | 1-15-2 |
| 154 | GTCTTGGTACTAGGCTG | 154_1 | GtcttggtactaggcTG | 1-14-2 |
| 155 | GGTCTTGGTACTAGGCTG | 155_1 | GgtcttggtactaggcTG | 1-15-2 |
| 156 | GTCTTGGTACTAGGCT | 156_1 | GtcttggtactagGCT | 1-12-3 |
| 156 | GTCTTGGTACTAGGCT | 156_2 | GtcttggtactaggCT | 1-13-2 |
| 157 | GGTCTTGGTACTAGGCT | 157_1 | GgtcttggtactaggCT | 1-14-2 |
| 158 | GGTCTTGGTACTAGGC | 158_1 | GGtcttggtactagGC | 2-12-2 |
| 159 | CAGGATGTCCAGGAGCAC | 159_1 | CaggatgtccaggagcAC | 1-15-2 |
| 160 | CGAGGAAGGCCACGTAGC | 160_1 | Cgaggaaggcca^{m}cgtaGC | 1-15-4 |
| | CC | | CC | |
| 161 | CGAGGAAGGCCACGTAG | 161_1 | Cgaggaaggcca^{m}cgtAG | 1-14-2 |
| 162 | CTCGAGGAAGGCCACGT | 162_1 | Ct^{m}cgaggaaggccacGT | 1-14-2 |
| 163 | CTCGAGGAAGGCCACG | 163_1 | Ct^{m}cgaggaaggccaCG | 1-13-2 |
| 164 | CTCTCGAGGAAGGCCAC | 164_1 | Ctct^{m}cgaggaaggccAC | 1-14-2 |
| 165 | AGTAGAGCTCCAGGCTC | 165_1 | AgtagagctccaggcTC | 1-14-2 |
| 166 | TAGTAGAGCTCCAGGCTC | 166_1 | TagtagagctccaggcTC | 1-15-2 |
| 167 | TAGTAGAGCTCCAGGCT | 167_1 | TagtagagctccaggCT | 1-14-2 |
| 168 | CGGGTAGTAGAGCTCCAG | 168_1 | CgggtagtagagctccAG | 1-15-2 |
| 169 | CGGGTAGTAGAGCTCCA | 169_1 | CgggtagtagagctcCA | 1-14-2 |
| 170 | CGGGTAGTAGAGCTCC | 170_1 | CgggtagtagagcTCC | 1-12-3 |
| 170 | CGGGTAGTAGAGCTCC | 170_2 | CgggtagtagagctCC | 1-13-2 |
| 171 | GCGGGTAGTAGAGCTC | 171_1 | G^{m}cgggtagtagagCTC | 1-12-3 |
| 171 | GCGGGTAGTAGAGCTC | 171_2 | G^{m}cgggtagtagagcTC | 1-13-2 |
| 172 | TGCGGGTAGTAGAGCTC | 172_1 | TG^{m}cgggtagtagagcTC | 2-13-2 |
| 172 | TGCGGGTAGTAGAGCTC | 172_2 | Tg^{m}cgggtagtagagcTC | 1-14-2 |
| 173 | CTGCGGGTAGTAGAGCTC | 173_1 | Ctg^{m}cgggtagtagagcTC | 1-15-2 |
| 174 | GCTGCGGGTAGTAGAGCT | 174_1 | GCtg^{m}cgggtagtagagcTC | 2-15-2 |
| | C | | | |
| 175 | TGCGGGTAGTAGAGCT | 175_1 | Tg^{m}cgggtagtagaGCT | 1-12-3 |
| 175 | TGCGGGTAGTAGAGCT | 175_2 | TG^{m}cgggtagtagagCT | 2-12-2 |
| 175 | TGCGGGTAGTAGAGCT | 175_3 | Tg^{m}cgggtagtagagCT | 1-13-2 |
| 176 | CTGCGGGTAGTAGAGCT | 176_1 | Ctg^{m}cgggtagtagagCT | 1-14-2 |
| 177 | CTGCGGGTAGTAGAGC | 177_1 | CTg^{m}cgggtagtagaGC | 2-12-2 |
| 177 | CTGCGGGTAGTAGAGC | 177_2 | Ctg^{m}cgggtagtagaGC | 1-13-2 |
| 178 | GCTGCGGGTAGTAGAGC | 178_1 | Gctg^{m}cgggtagtagaGC | 1-14-2 |
| 179 | AGCTGCGGGTAGTAGAGC | 179_1 | AGCtg^{m}cgggtagtagaGC | 3-13-2 |
| 180 | GCTGCGGGTAGTAGAG | 180_1 | Gctg^{m}cgggtagtaGAG | 1-12-3 |
| 180 | GCTGCGGGTAGTAGAG | 180_2 | Gctg^{m}cgggtagtagAG | 1-13-2 |
| 181 | AGCTGCGGGTAGTAGAG | 181_1 | Agctg^{m}cgggtagtagAG | 1-14-2 |
| 182 | GCTGCGGGTAGTAGA | 182_1 | GCtg^{m}cgggtagtaGA | 2-11-2 |
| 182 | GCTGCGGGTAGTAGA | 182_2 | Gctg^{m}cgggtagtAGA | 1-11-3 |
| 182 | GCTGCGGGTAGTAGA | 182_3 | Gctg^{m}cgggtagtaGA | 1-12-2 |
| 183 | AGCTGCGGGTAGTAGA | 183_1 | AGctg^{m}cgggtagtaGA | 2-12-2 |
| 183 | AGCTGCGGGTAGTAGA | 183_2 | Agctg^{m}cgggtagtaGA | 1-13-2 |
| 184 | TGACCTTCTTGTACAGCTG | 184_1 | TgaccttcttgtacagcTG | 1-16-2 |
| 185 | GACCTTCTTGTACAGCT | 185_1 | GaccttcttgtacagCT | 1-14-2 |
| 186 | TGACCTTCTTGTACAGCT | 186_1 | TGaccttcttgtacagCT | 2-14-2 |
| 186 | TGACCTTCTTGTACAGCT | 186_2 | TgaccttcttgtacagCT | 1-15-2 |
| 187 | TGACCTTCTTGTACAGC | 187_1 | TgaccttcttgtacAGC | 1-13-3 |
| 187 | TGACCTTCTTGTACAGC | 187_2 | TgaccttcttgtacaGC | 1-14-2 |
| 188 | TCATGGAGAAGACGCG | 188_1 | TCatggagaagaCGCG | 2-10-4 |
| 188 | TCATGGAGAAGACGCG | 188_2 | TCATggagaaga^{m}cgCG | 4-10-2 |
| 188 | TCATGGAGAAGACGCG | 188_3 | TCatggagaagacGCG | 2-11-3 |
| 189 | ATCATGGAGAAGACGCG | 189_1 | ATCAtggagaaga^{m}cgCG | 4-11-2 |
| 190 | GATCATGGAGAAGACGCG | 190_1 | GATCatggagaaga^{m}cgCG | 4-12-2 |
| 190 | GATCATGGAGAAGACGCG | 190_2 | Gatcatggagaaga^{m}cgCG | 1-15-2 |
| 191 | TGATCATGGAGAAGACGC | 191_1 | Tgatcatggagaaga^{m}cgCG | 1-16-2 |
| | G | | | |
| 192 | ATGATCATGGAGAAGACG | 192_1 | AtgatcatggagaagacGCG | 1-16-3 |
| | CG | | | |
| 192 | ATGATCATGGAGAAGACG | 192_2 | Atgatcatggagaaga^{m}cgCG | 1-17-2 |
| | CG | | | |
| 193 | GATCATGGAGAAGACGC | 193_1 | GATCatggagaagacGC | 4-11-2 |
| 193 | GATCATGGAGAAGACGC | 193_2 | GatcatggagaagACGC | 1-12-4 |
| 194 | TGATCATGGAGAAGACGC | 194_1 | TgatcatggagaagACGC | 1-13-4 |
| 194 | TGATCATGGAGAAGACGC | 194_2 | TgatcatggagaagacGC | 1-15-2 |
| 195 | ATGATCATGGAGAAGACG | 195_1 | AtgatcatggagaagACGC | 1-14-4 |
| | C | | | |
| 195 | ATGATCATGGAGAAGACG | 195_2 | AtgatcatggagaagaCGC | 1-15-3 |
| | C | | | |
| 195 | ATGATCATGGAGAAGACG | 195_3 | ATgatcatggagaagacGC | 2-15-2 |
| | C | | | |
| 196 | TGATCATGGAGAAGACG | 196_1 | TGAtcatggagaagACG | 3-11-3 |
| 197 | ATGATCATGGAGAAGACG | 197_1 | ATGAtcatggagaagACG | 4-11-3 |
| 197 | ATGATCATGGAGAAGACG | 197_2 | ATGatcatggagaagACG | 3-12-3 |
| 198 | CCGATGATCATGGAGAAG | 198_1 | C^{m}cgatgatcatggagaagAC | 1-17-2 |
| | AC | | | |
| 199 | CGATGATCATGGAGAAGA | 199_1 | CgatgatcatggagAAGA | 1-13-4 |
| 200 | CCGATGATCATGGAGAAG | 200_1 | C^{m}cgatgatcatggagaaGA | 1-16-2 |
| | A | | | |
| 201 | ACCGATGATCATGGAGAA | 201_1 | AC^{m}cgatgatcatggagaAG | 2-15-2 |
| | G | | | |
| 202 | CACCGATGATCATGGAGA | 202_1 | Cac^{m}cgatgatcatggagaAG | 1-17-2 |
| | AG | | | |
| 203 | ACCGATGATCATGGAGAA | 203_1 | AC^{m}cgatgatcatggaGAA | 2-13-3 |
| 204 | CACCGATGATCATGGAGA | 204_1 | CAc^{m}cgatgatcatggagAA | 2-15-2 |
| | A | | | |
| 205 | CCGATGATCATGGAGA | 205_1 | C^{m}cgatgatcatggAGA | 1-12-3 |
| 206 | ACCGATGATCATGGAGA | 206_1 | Ac^{m}cgatgatcatggAGA | 1-13-3 |
| 207 | ACCGATGATCATGGAG | 207_1 | ACCgatgatcatggAG | 3-11-2 |
| 207 | ACCGATGATCATGGAG | 207_2 | AC^{m}cgatgatcatgGAG | 2-11-3 |
| 208 | CACCGATGATCATGGAG | 208_1 | CAc^{m}cgatgatcatggAG | 2-13-2 |
| 209 | TCACCGATGATCATGGAG | 209_1 | TCac^{m}cgatgatcatggAG | 2-14-2 |
| 210 | CACCGATGATCATGGA | 210_1 | CAC^{m}cgatgatcatGGA | 3-10-3 |
| 210 | CACCGATGATCATGGA | 210_2 | CAC^{m}cgatgatcatgGA | 3-11-2 |
| 211 | TCACCGATGATCATGG | 211_1 | TCAc^{m}cgatgatcaTGG | 3-10-3 |
| 211 | TCACCGATGATCATGG | 211_2 | TCac^{m}cgatgatcaTGG | 2-11-3 |
| 212 | CTCACCGATGATCATGG | 212_1 | Ctcac^{m}cgatgatcaTGG | 1-13-3 |
| 213 | ACTCACCGATGATCATG | 213_1 | ACtcac^{m}cgatgatCATG | 2-11-4 |
| 213 | ACTCACCGATGATCATG | 213_2 | ACtcac^{m}cgatgatcATG | 2-12-3 |
| 214 | CACTCACCGATGATCATG | 214_1 | CACtcac^{m}cgatgatcaTG | 3-13-2 |
| 214 | CACTCACCGATGATCATG | 214_2 | Cactcac^{m}cgatgatcaTG | 1-15-2 |
| 215 | ACTCACCGATGATCAT | 215_1 | ACTCac^{m}cgatgatCAT | 4-9-3 |
| 215 | ACTCACCGATGATCAT | 215_2 | ACTCac^{m}cgatgatcAT | 4-10-2 |
| 216 | CACTCACCGATGATCAT | 216_1 | CACtcac^{m}cgatgatCAT | 3-11-3 |
| 216 | CACTCACCGATGATCAT | 216_2 | Cactcac^{m}cgatgatCAT | 1-13-3 |
| 217 | TCACTCACCGATGATCAT | 217_1 | TCactcac^{m}cgatgatcAT | 2-14-2 |
| 218 | TCACTCACCGATGATCA | 218_1 | TCactcac^{m}cgatgatCA | 2-13-2 |
| 219 | GTCACTCACCGATGATCA | 219_1 | Gtcactcac^{m}cgatgaTCA | 1-14-3 |
| 220 | TCACTCACCGATGATC | 220_1 | TCactcac^{m}cgatgaTC | 2-12-2 |
| 221 | GTCACTCACCGATGATC | 221_1 | Gtcactcac^{m}cgatgaTC | 1-14-2 |
| 222 | AGGGCTCCTAGGGATGG | 222_1 | AgggctcctagggatGG | 1-14-2 |
| 223 | AGCTCCTTGATGAAGTCAT | 223_1 | AGCtccttgatgaagtCATC | 3-13-4 |
| | C | | | |
| 224 | AGCTCCTTGATGAAGTCAT | 224_1 | AgctccttgatgaagtCAT | 1-15-3 |
| 225 | CAGCTCCTTGATGAAGTCA | 225_1 | CAGCtccttgatgaagtCAT | 4-13-3 |
| | T | | | |
| 226 | AGGTCGTAGTTCTCCTC | 226_1 | Aggt^{m}cgtagttctccTC | 1-14-2 |
| 227 | GGCCAGGTCGTAGTTC | 227_1 | Ggccaggt^{m}cgtagTTC | 1-12-3 |
| 228 | TGGCCAGGTCGTAGTTC | 228_1 | Tggccaggt^{m}cgtagtTC | 1-14-2 |
| 229 | ATGGCCAGGTCGTAGTTC | 229_1 | Atggccaggt^{m}cgtagtTC | 1-15-2 |
| 230 | TGGCCAGGTCGTAGTT | 230_1 | TGgccaggt^{m}cgtagTT | 2-12-2 |
| 231 | ATGGCCAGGTCGTAGTT | 231_1 | Atggccaggt^{m}cgtagTT | 1-14-2 |
| 232 | CATGGCCAGGTCGTAGTT | 232_1 | Catggccaggt^{m}cgtagTT | 1-15-2 |
| 233 | CATGGCCAGGTCGTAG | 233_1 | Catggccaggt^{m}cgtAG | 1-13-2 |
| 234 | GTTCCGCATGAGCG | 234_1 | GTTC^{m}cgcatgagCG | 4-8-2 |
| 235 | CGGTTCCGCATGAGCG | 235_1 | Cggttc^{m}cgcatgagCG | 1-13-2 |
| 236 | CGGTTCCGCATGAGC | 236_1 | Cggttc^{m}cgcatgAGC | 1-11-3 |
| 237 | ACGGTTCCGCATGAG | 237_1 | A^{m}cggttc^{m}cgcatgAG | 1-12-2 |
| 238 | CACGGTTCCGCATGAG | 238_1 | Ca^{m}cggttc^{m}cgcatgAG | 1-13-2 |
| 239 | GTCACGGTTCCGCAT | 239_1 | Gtca^{m}cggttc^{m}cgcAT | 1-12-2 |
| 240 | GGTCACGGTTCCGCAT | 240_1 | Ggtca^{m}cggttc^{m}cgcAT | 1-13-2 |
| 241 | AAGGGGGCAAAAGGCAAT | 241_1 | AagggggcaaaaggcAATG | 1-14-4 |
| | G | | | |
| 242 | AAGGGGGCAAAAGGCAAT | 242_1 | AAgggggcaaaaggCAAT | 2-12-4 |
| 242 | AAGGGGGCAAAAGGCAAT | 242_2 | AAGggggcaaaaggcaAT | 3-13-2 |
| 242 | AAGGGGGCAAAAGGCAAT | 242_3 | AagggggcaaaaggCAAT | 1-13-4 |
| 243 | GAAGGGGGCAAAAGGCAA | 243_1 | GaagggggcaaaaggCAAT | 1-14-4 |
| | T | | | |
| 243 | GAAGGGGGCAAAAGGCAA | 243_2 | GaagggggcaaaaggcAAT | 1-15-3 |
| | T | | | |
| 244 | TGAAGGGGGCAAAAGGCA | 244_1 | TgaagggggcaaaaggcAA | 1-16-3 |
| | AT | | T | |
| 245 | GAAGGGGGCAAAAGGCAA | 245_1 | GAagggggcaaaaggCAA | 2-13-3 |
| 245 | GAAGGGGGCAAAAGGCAA | 245_2 | GaagggggcaaaaggCAA | 1-14-3 |
| 246 | TGAAGGGGGCAAAAGGCA | 246_1 | TGaagggggcaaaaggCAA | 2-14-3 |
| | A | | | |
| 246 | TGAAGGGGGCAAAAGGCA | 246_2 | TgaagggggcaaaaggCAA | 1-15-3 |
| | A | | | |
| 247 | CTGAAGGGGGCAAAAGGC | 247_1 | CtgaagggggcaaaaggcAA | 1-17-2 |
| | AA | | | |
| 248 | TGAAGGGGGCAAAAGGCA | 248_1 | TgaagggggcaaaaggCA | 1-15-2 |
| 249 | CTGAAGGGGGCAAAAGGC | 249_1 | CtgaagggggcaaaaggCA | 1-16-2 |
| | A | | | |
| 250 | TGAAGGGGGCAAAAGGC | 250_1 | TgaagggggcaaaagGC | 1-14-2 |
| 251 | CTGAAGGGGGCAAAAGGC | 251_1 | CtgaagggggcaaaagGC | 1-15-2 |
| 252 | TCCTGAAGGGGGCAAAAG | 252_1 | TCctgaagggggcaAAAG | 2-12-4 |
| 252 | TCCTGAAGGGGGCAAAAG | 252_2 | TCctgaagggggcaaaAG | 2-14-2 |
| 253 | CTCCTGAAGGGGGCAAAA | 253_1 | CtcctgaagggggcaaaAG | 1-16-2 |
| | G | | | |
| 254 | CTCCTGAAGGGGGCAAAA | 254_1 | CtcctgaagggggcaAAA | 1-14-3 |
| 255 | CTCCTGAAGGGGGCAAA | 255_1 | CTCctgaagggggcAAA | 3-11-3 |
| 255 | CTCCTGAAGGGGGCAAA | 255_2 | CtcctgaagggggCAAA | 1-12-4 |
| 255 | CTCCTGAAGGGGGCAAA | 255_3 | CtcctgaagggggcaAA | 1-14-2 |
| 256 | GGATGTAGGGGCTGCTC | 256_1 | GgatgtaggggctgcTC | 1-14-2 |
| 257 | CTGGATGTAGGGGCTGC | 257_1 | CtggatgtaggggctGC | 1-14-2 |
| 258 | GTACCTGGATGTAGGGGC | 258_1 | GtacctggatgtagGGGC | 1-13-4 |
| 259 | AAGATGGTGTTGGCCTG | 259_1 | AagatggtgttggccTG | 1-14-2 |
| 260 | GGGAGAAGATGGTGTTGG | 260_1 | GggagaagatggtgttggCC | 1-17-2 |
| | CC | | | |
| 261 | GGAGAAGATGGTGTTGGC | 261_1 | GgagaagatggtgttgGC | 1-15-2 |
| 262 | GCGCAGGGAGAAGATGGT | 262_1 | G^{m}cgcagggagaagatgGT | 1-15-2 |
| 263 | TGCGCAGGGAGAAGATG | 263_1 | TG^{m}cgcagggagaagATG | 2-12-3 |
| 263 | TGCGCAGGGAGAAGATG | 263_2 | TG^{m}cgcagggagaagaTG | 2-13-2 |
| 264 | TTGCGCAGGGAGAAGATG | 264_1 | TTG^{m}cgcagggagaagaTG | 3-13-2 |
| 265 | CTTGCGCAGGGAGAAGAT | 265_1 | Cttg^{m}cgcagggagaagAT | 1-15-2 |
| 266 | CCTTGCGCAGGGAGAAG | 266_1 | Ccttg^{m}cgcagggagAAG | 1-13-3 |
| 266 | CCTTGCGCAGGGAGAAG | 266_2 | Ccttg^{m}cgcagggagaAG | 1-14-2 |
| 267 | CCTTGCGCAGGGAGAA | 267_1 | Ccttg^{m}cgcagggagAA | 1-13-2 |
| 268 | TCCTTGCGCAGGGAGAA | 268_1 | TCcttg^{m}cgcagggagAA | 2-13-2 |
| 269 | TGGCGGAGGTCCTTGC | 269_1 | TGg^{m}cggaggtccTTGC | 2-10-4 |
| 270 | CTGGCGGAGGTCCTTG | 270_1 | Ctgg^{m}cggaggtccTTG | 1-12-3 |
| 271 | GGAGGCGTCGGGCCTCG | 271_1 | Ggagg^{m}cgt^{m}cgggccTCG | 1-13-4 |
| | C | | C | |
| 272 | CGGAGGCGTCGGGCCTC | 272_1 | Cggagg^{m}cgt^{m}cgggccTCG | 1-14-4 |
| | GC | C | | |
| 273 | CGGAGGCGTCGGGCCTC | 273_1 | CGgagg^{m}cgt^{m}cgggccTC | 2-13-3 |
| | G | | G | |
| 274 | CCGGAGGCGTCGGGCCT | 274_1 | CCGgagg^{m}cgt^{m}cgggCCT | 3-11-3 |
| 275 | ACCCGGAGGCGTCGGGC | 275_1 | ACC^{m}cggagg^{m}cgt^{m}cggg | 3-13-2 |
| | C | | CC | |
| 276 | CTACCCGGAGGCGTCGGG | 276_1 | Ctacc^{m}cggagg^{m}cgt^{m}cggG | 1-16-2 |
| | C | | C | |
| 277 | CCTACCCGGAGGCGTCGG | 277_1 | Cctacc^{m}cggagg^{m}cgt^{m}cgg | 1-17-2 |
| | GC | | GC | |
| 278 | CTACCCGGAGGCGTCGGG | 278_1 | Ctacc^{m}cggagg^{m}cgtCGG | 1-13-4 |
| | | | G | |
| 279 | CTACCCGGAGGCGTCGG | 279_1 | Ctacc^{m}cggagg^{m}cgtCGG | 1-13-3 |
| 280 | TACCCGGAGGCGTCG | 280_1 | Tacc^{m}cggagg^{m}cgTCG | 1-11-3 |
| 280 | TACCCGGAGGCGTCG | 280_2 | Tacc^{m}cggagg^{m}cgtCG | 1-12-2 |
| 281 | CTACCCGGAGGCGTCG | 281_1 | Ctacc^{m}cggagg^{m}cgtCG | 1-13-2 |
| 282 | CCTACCCGGAGGCGTCG | 282_1 | CCtacc^{m}cggagg^{m}cgtCG | 2-13-2 |
| 283 | AGGCACGGAGTGGGCG | 283_1 | Aggca^{m}cggagtgggCG | 1-13-2 |
| 284 | CAGCTCGAACATCTCCT | 284_1 | Cagct^{m}cgaacatctcCT | 1-14-2 |
| 285 | TGTACATCTTGGAGTCCTT | 285_1 | TGtacatcttggagtccTT | 2-15-2 |
| 285 | TGTACATCTTGGAGTCCTT | 285_2 | TgtacatcttggagtccTT | 1-16-2 |
| 286 | TTGTACATCTTGGAGTCCT | 286_1 | TtgtacatcttggagtccTT | 1-17-2 |
| | T | | | |
| 287 | TGTACATCTTGGAGTCCT | 287_1 | TGtacatcttggagtcCT | 2-14-2 |
| 287 | TGTACATCTTGGAGTCCT | 287_2 | TgtacatcttggagtcCT | 1-15-2 |
| 288 | TTGTACATCTTGGAGTCCT | 288_1 | TTgtacatcttggagtcCT | 2-15-2 |
| 288 | TTGTACATCTTGGAGTCCT | 288_2 | TtgtacatcttggagtcCT | 1-16-2 |
| 289 | TGTACATCTTGGAGTCC | 289_1 | TgtacatcttggagTCC | 1-13-3 |
| 289 | TGTACATCTTGGAGTCC | 289_2 | TgtacatcttggagtCC | 1-14-2 |
| 290 | TTGTACATCTTGGAGTCC | 290_1 | TtgtacatcttggagTCC | 1-14-3 |
| 290 | TTGTACATCTTGGAGTCC | 290_2 | TtgtacatcttggagtCC | 1-15-2 |
| 291 | CTTGTACATCTTGGAGTCC | 291_1 | CttgtacatcttggagtCC | 1-16-2 |
| 292 | TTGTACATCTTGGAGTC | 292_1 | TtgtacatcttggAGTC | 1-12-4 |
| 292 | TTGTACATCTTGGAGTC | 292_2 | TtgtacatcttggaGTC | 1-13-3 |
| 293 | CTTGTACATCTTGGAGTC | 293_1 | CTtgtacatcttggagTC | 2-14-2 |
| 294 | CCTTGTACATCTTGGAGT | 294_1 | CcttgtacatcttggaGT | 1-15-2 |
| 295 | TCCTTGTACATCTTGGAG | 295_1 | TCcttgtacatcttggAG | 2-14-2 |
| 296 | TCCTTGTACATCTTGGA | 296_1 | TCcttgtacatcttGGA | 2-12-3 |
| 296 | TCCTTGTACATCTTGGA | 296_2 | TCcttgtacatcttgGA | 2-13-2 |
| 297 | GTCCTTGTACATCTTGGA | 297_1 | GtccttgtacatcttgGA | 1-15-2 |
| 298 | TGCGGTCCTTGTACATC | 298_1 | Tg^{m}cggtccttgtacaTC | 1-14-2 |
| 299 | ATGCGGTCCTTGTACATC | 299_1 | Atg^{m}cggtccttgtacaTC | 1-15-2 |
| 300 | GATGCGGTCCTTGTACATC | 300_1 | Gatg^{m}cggtccttgtacaTC | 1-16-2 |
| 301 | TGCGGTCCTTGTACAT | 301_1 | TG^{m}cggtccttgtacAT | 2-12-2 |
| 302 | GATGCGGTCCTTGTACAT | 302_1 | Gatg^{m}cggtccttgtacAT | 1-15-2 |
| 303 | CGATGCGGTCCTTGTACAT | 303_1 | Cgatg^{m}cggtccttgtacAT | 1-16-2 |
| 304 | GATGCGGTCCTTGTACA | 304_1 | GAtg^{m}cggtccttgtaCA | 2-13-2 |
| 304 | GATGCGGTCCTTGTACA | 304_2 | Gatg^{m}cggtccttgtaCA | 1-14-2 |
| 305 | CGATGCGGTCCTTGTACA | 305_1 | Cgatg^{m}cggtccttgtaCA | 1-15-2 |
| 306 | AGGATGGCCTCGATGCG | 306_1 | Aggatggcct^{m}cgatgCG | 1-14-2 |
| 307 | CAAAAACAAATAACAAAGA | 307_1 | CAAAaacaaataacaaAGA | 4-12-4 |
| | T | | T | |
| 308 | GAAGTATTGACTTCATC | 308_1 | GAAGtattgacttCATC | 4-9-4 |
| 309 | GGAAGTATTGACTTCAT | 309_1 | GGAAgtattgacttCAT | 4-10-3 |
| 310 | GGGAAGTATTGACTTCAT | 310_1 | GGGAagtattgacttcAT | 4-12-2 |
| 311 | TTGAACACGGTTTTCCCT | 311_1 | Ttgaaca^{m}cggttttccCT | 1-15-2 |
| 312 | TTGAACACGGTTTTCCC | 312_1 | Ttgaaca^{m}cggttttcCC | 1-14-2 |
| 313 | GTTGAACACGGTTTTCCC | 313_1 | Gttgaaca^{m}cggttttcCC | 1-15-2 |
| 314 | AGGTTGAACACGGTTTTCC | 314_1 | Aggttgaaca^{m}cggttttCC | 1-16-2 |
| 315 | GAAGGTTGAACACGGTTTT | 315_1 | GAaggttgaaca^{m}cggttTTC | 2-15-3 |
| | C | | | |
| 316 | AAGTCTGTAAGGTGGAGC | 316_1 | AagtctgtaaggtggaGC | 1-15-2 |
| 317 | AAAACAAGACAGAATGTTT | 317_1 | AAAAcaagacagaatGTTT | 4-11-4 |
| 318 | TAAAACAAGACAGAATGTT | 318_1 | TAAAacaagacagaatGTT | 4-12-4 |
| | T | | T | |
| 319 | GTAAAACAAGACAGAATGT | 319_1 | GTaaaacaagacagaaTGT | 2-14-4 |
| | T | | T | |
| 320 | GGTAAAACAAGACAGAAT | 320_1 | GGTAaaacaagacagaatG | 4-14-2 |
| | GT | | T | |
| 320 | GGTAAAACAAGACAGAAT | 320_2 | GGTaaaacaagacagaatG | 3-15-2 |
| | GT | | T | |
| 321 | TGGTAAAACAAGACAGAAT | 321_1 | TGGTaaaacaagacagaaT | 4-14-2 |
| | G | | G | |
| 322 | TGGTAAAACAAGACAGAAT | 322_1 | TGGTaaaacaagacagAA | 4-12-3 |
| | T | | | |
| 323 | CTGGTAAAACAAGACAGAA | 323_1 | CTGGtaaaacaagacaGA | 4-12-4 |
| | T | | AT | |
| 323 | CTGGTAAAACAAGACAGAA | 323_2 | CTGGtaaaacaagacagAA | 4-13-3 |
| | T | | T | |
| 323 | CTGGTAAAACAAGACAGAA | 323_3 | CTggtaaaacaagacaGAA | 2-14-4 |
| | T | | T | |
| 324 | CTGGTAAAACAAGACAGAA | 324_1 | CTGGtaaaacaagacAGA | 4-11-4 |
| | A | | | |
| 324 | CTGGTAAAACAAGACAGAA | 324_2 | CTGGtaaaacaagacaGA | 4-12-3 |
| | A | | | |
| 324 | CTGGTAAAACAAGACAGAA | 324_3 | CTGgtaaaacaagacaGAA | 3-13-3 |
| 325 | ACTGGTAAAACAAGACAGA | 325_1 | ACTGgtaaaacaagacAGA | 4-12-4 |
| | A | | A | |
| 325 | ACTGGTAAAACAAGACAGA | 325_2 | ACTggtaaaacaagacAGA | 3-13-4 |
| | A | | A | |
| 325 | ACTGGTAAAACAAGACAGA | 325_3 | ACTggtaaaacaagacaGA | 3-14-3 |
| | A | | A | |
| 326 | CTGGTAAAACAAGACAGA | 326_1 | CTGgtaaaacaagaCAGA | 3-11-4 |
| 326 | CTGGTAAAACAAGACAGA | 326_2 | CTGGtaaaacaagacaGA | 4-12-2 |
| 326 | CTGGTAAAACAAGACAGA | 326_3 | CTGgtaaaacaagacAGA | 3-12-3 |
| 327 | ACTGGTAAAACAAGACAGA | 327_1 | ACTggtaaaacaagaCAGA | 3-12-4 |
| 327 | ACTGGTAAAACAAGACAGA | 327_2 | ACTGgtaaaacaagacaGA | 4-13-2 |
| 328 | TACTGGTAAAACAAGACAG | 328_1 | TACTggtaaaacaagacAG | 4-13-3 |
| | A | | A | |
| 328 | TACTGGTAAAACAAGACAG | 328_2 | TACtggtaaaacaagacaGA | 3-15-2 |
| | A | | | |
| 329 | ACTGGTAAAACAAGACAG | 329_1 | ACTGgtaaaacaagACAG | 4-10-4 |
| 329 | ACTGGTAAAACAAGACAG | 329_2 | ACTGgtaaaacaagaCAG | 4-11-3 |
| 329 | ACTGGTAAAACAAGACAG | 329_3 | ACTggtaaaacaagACAG | 3-11-4 |
| 330 | TACTGGTAAAACAAGACAG | 330_1 | TACTggtaaaacaagACA | 4-11-4 |
| | | | G | |
| 330 | TACTGGTAAAACAAGACAG | 330_2 | TACTggtaaaacaagaCAG | 4-12-3 |
| 330 | TACTGGTAAAACAAGACAG | 330_3 | TACTggtaaaacaagacAG | 4-13-2 |
| 331 | CTACTGGTAAAACAAGACA | 331_1 | CTACtggtaaaacaagacA | 4-14-2 |
| | G | | G | |
| 331 | CTACTGGTAAAACAAGACA | 331_2 | CtactggtaaaacaagACAG | 1-15-4 |
| | G | | | |
| 332 | TACTGGTAAAACAAGACA | 332_1 | TACTggtaaaacaaGACA | 4-10-4 |
| 332 | TACTGGTAAAACAAGACA | 332_2 | TACTggtaaaacaagACA | 4-11-3 |
| 333 | CTACTGGTAAAACAAGACA | 333_1 | CTActggtaaaacaagACA | 3-13-3 |
| 334 | GCTACTGGTAAAACAAGAC | 334_1 | GCtactggtaaaacaagACA | 2-15-3 |
| | A | | | |
| 334 | GCTACTGGTAAAACAAGAC | 334_2 | GCtactggtaaaacaagaCA | 2-16-2 |
| | A | | | |
| 334 | GCTACTGGTAAAACAAGAC | 334_3 | GctactggtaaaacaagACA | 1-16-3 |
| | A | | | |
| 335 | TACTGGTAAAACAAGAC | 335_1 | TACTggtaaaacaAGAC | 4-9-4 |
| 336 | CTACTGGTAAAACAAGAC | 336_1 | CTACtggtaaaacaAGAC | 4-10-4 |
| 337 | GCTACTGGTAAAACAAGAC | 337_1 | GCtactggtaaaacaaGAC | 2-14-3 |
| 337 | GCTACTGGTAAAACAAGAC | 337_2 | GctactggtaaaacaAGAC | 1-14-4 |
| 338 | AGCTACTGGTAAAACAAGA | 338_1 | AGctactggtaaaacaAGAC | 2-14-4 |
| | C | | | |
| 338 | AGCTACTGGTAAAACAAGA | 338_2 | AgctactggtaaaacaAGAC | 1-15-4 |
| | C | | | |
| 338 | AGCTACTGGTAAAACAAGA | 338_3 | AgctactggtaaaacaaGAC | 1-16-3 |
| | C | | | |
| 339 | GCTACTGGTAAAACAAGA | 339_1 | GCTactggtaaaacAAGA | 3-11-4 |
| 339 | GCTACTGGTAAAACAAGA | 339_2 | GCtactggtaaaacAAGA | 2-12-4 |
| 339 | GCTACTGGTAAAACAAGA | 339_3 | GCtactggtaaaacaAGA | 2-13-3 |
| 340 | AGCTACTGGTAAAACAAGA | 340_1 | AGCtactggtaaaacaAGA | 3-13-3 |
| 340 | AGCTACTGGTAAAACAAGA | 340_2 | AGCtactggtaaaacaaGA | 3-14-2 |
| 340 | AGCTACTGGTAAAACAAGA | 340_3 | AGctactggtaaaacaAGA | 2-14-3 |
| 341 | AAGCTACTGGTAAAACAAG | 341_1 | AAGCtactggtaaaacaaGA | 4-14-2 |
| | A | | | |
| 341 | AAGCTACTGGTAAAACAAG | 341_2 | AAGctactggtaaaacAAGA | 3-13-4 |
| | A | | | |
| 341 | AAGCTACTGGTAAAACAAG | 341_3 | AAgctactggtaaaacAAGA | 2-14-4 |
| | A | | | |
| 342 | GCTACTGGTAAAACAAG | 342_1 | GCTActggtaaaaCAAG | 4-9-4 |
| 342 | GCTACTGGTAAAACAAG | 342_2 | GCTactggtaaaaCAAG | 3-10-4 |
| 342 | GCTACTGGTAAAACAAG | 342_3 | GCTActggtaaaacAAG | 4-10-3 |
| 343 | AGCTACTGGTAAAACAAG | 343_1 | AGCtactggtaaaaCAAG | 3-11-4 |
| 343 | AGCTACTGGTAAAACAAG | 343_2 | AGCTactggtaaaacaAG | 4-12-2 |
| 343 | AGCTACTGGTAAAACAAG | 343_3 | AGCtactggtaaaacAAG | 3-12-3 |
| 344 | AAGCTACTGGTAAAACAAG | 344_1 | AAGCtactggtaaaacAAG | 4-12-3 |
| 344 | AAGCTACTGGTAAAACAAG | 344_2 | AAGctactggtaaaaCAAG | 3-12-4 |
| 345 | AAAGCTACTGGTAAAACAA | 345_1 | AAAGctactggtaaaaCAA | 4-12-4 |
| | G | | G | |
| 345 | AAAGCTACTGGTAAAACAA | 345_2 | AAAgctactggtaaaaCAAG | 3-13-4 |
| | G | | | |
| 346 | AAGCTACTGGTAAAACAA | 346_1 | AAGCtactggtaaaACAA | 4-10-4 |
| 346 | AAGCTACTGGTAAAACAA | 346_2 | AAGCtactggtaaaaCAA | 4-11-3 |
| 347 | AAAGCTACTGGTAAAACAA | 347_1 | AAAGctactggtaaaACAA | 4-11-4 |
| 348 | AAAAGCTACTGGTAAAACA | 348_1 | AAAAgctactggtaaaACAA | 4-12-4 |
| | A | | | |
| 349 | AAAGCTACTGGTAAAACA | 349_1 | AAAGctactggtaaAACA | 4-10-4 |
| 350 | AAAAGCTACTGGTAAAACA | 350_1 | AAAAgctactggtaaAACA | 4-11-4 |
| 351 | AAAAAGCTACTGGTAAAAC | 351_1 | AAAAagctactggtaaAACA | 4-12-4 |
| | A | | | |
| 352 | AAAAAAAGCTACTGGTAA | 352_1 | AAAAaaagctactgGTAA | 4-10-4 |
| 353 | TTAAAAAAAGCTACTGGT | 353_1 | TTAAaaaaagctacTGGT | 4-10-4 |
| 354 | ATTAAAAAAAGCTACTGGT | 354_1 | ATTaaaaaaagctacTGGT | 3-12-4 |
| 355 | GATTAAAAAAAGCTACTGG | 355_1 | GATTaaaaaaagctactGG | 4-13-3 |
| | T | | T | |
| 355 | GATTAAAAAAAGCTACTGG | 355_2 | GATtaaaaaaagctactGGT | 3-14-3 |
| | T | | | |
| 356 | ATTAAAAAAAGCTACTGG | 356_1 | ATTAaaaaaagctaCTGG | 4-10-4 |
| 357 | GATTAAAAAAAGCTACTGG | 357_1 | GATTaaaaaaagctacTGG | 4-12-3 |
| 358 | AGATTAAAAAAAGCTACTG | 358_1 | AGATtaaaaaaagctacTG | 4-13-3 |
| | G | | G | |
| 358 | AGATTAAAAAAAGCTACTG | 358_2 | AGAttaaaaaaagctacTGG | 3-14-3 |
| | G | | | |
| 359 | GATTAAAAAAAGCTACTG | 359_1 | GATTaaaaaaagctaCTG | 4-11-3 |
| 360 | AGATTAAAAAAAGCTACTG | 360_1 | AGATtaaaaaaagctACTG | 4-11-4 |
| 361 | AAGATTAAAAAAAGCTACT | 361_1 | AAGAttaaaaaaagctACT | 4-12-4 |
| | G | | G | |
| 362 | AAGATTAAAAAAAGCTACT | 362_1 | AAGAttaaaaaaagcTACT | 4-11-4 |
| 363 | AGCGCAATGGTGACTT | 363_1 | AG^{m}cgcaatggtgacTT | 2-12-2 |
| 364 | TTAAGGCTCCTGATGTGGA | 364_1 | TtaaggctcctgatgtgGA | 1-16-2 |
| 365 | TTTAAGGCTCCTGATGTGG | 365_1 | TttaaggctcctgatgtgGA | 1-17-2 |
| | A | | | |
| 366 | TTAAGGCTCCTGATGTGG | 366_1 | TtaaggctcctgatgtGG | 1-15-2 |
| 367 | TTTAAGGCTCCTGATGTGG | 367_1 | TttaaggctcctgatgtGG | 1-16-2 |
| 368 | TTAAGGCTCCTGATGTG | 368_1 | TtaaggctcctgatgTG | 1-14-2 |
| 369 | TTTAAGGCTCCTGATGTG | 369_1 | TttaaggctcctgatgTG | 1-15-2 |
| 370 | TTTTAAGGCTCCTGATGTG | 370_1 | TtttaaggctcctgatgTG | 1-16-2 |
| 371 | TCTCGTTTTAAGGCTCCTG | 371_1 | TCt^{m}cgttttaaggctccTG | 2-15-2 |
| 372 | GGTCTCGTTTTAAGGCT | 372_1 | Ggtct^{m}cgttttaaggCT | 1-14-2 |
| 373 | GGGGTCTCGTTTTAAGGC | 373_1 | Ggggtct^{m}cgttttaaggCT | 1-16-2 |
| | T | | | |
| 374 | CCAGGGGTCTCGTTTTAA | 374_1 | Ccaggggtct^{m}cgttttaaGG | 1-17-2 |
| | GG | | | |
| 375 | GGGGTCTCGTTTTAAG | 375_1 | GGggtct^{m}cgttttAAG | 2-11-3 |
| 375 | GGGGTCTCGTTTTAAG | 375_2 | GGggtct^{m}cgttttaAG | 2-12-2 |
| 376 | AGGGGTCTCGTTTTAAG | 376_1 | AGgggtct^{m}cgttttAAG | 2-12-3 |
| 376 | AGGGGTCTCGTTTTAAG | 376_2 | Aggggtct^{m}cgttttAAG | 1-13-3 |
| 377 | CAGGGGTCTCGTTTTAAG | 377_1 | Caggggtct^{m}cgttttAAG | 1-14-3 |
| 377 | CAGGGGTCTCGTTTTAAG | 377_2 | Caggggtct^{m}cgttttaAG | 1-15-2 |
| 378 | CCAGGGGTCTCGTTTTAA | 378_1 | Ccaggggtct^{m}cgttttaAG | 1-16-2 |
| | G | | | |
| 379 | CAGGGGTCTCGTTTTAA | 379_1 | Caggggtct^{m}cgttTTAA | 1-12-4 |
| 379 | CAGGGGTCTCGTTTTAA | 379_2 | CAggggtct^{m}cgttttAA | 2-13-2 |
| 380 | CCAGGGGTCTCGTTTTAA | 380_1 | Ccaggggtct^{m}cgttttAA | 1-15-2 |
| 381 | CCCAGGGGTCTCGTTTTAA | 381_1 | CCCaggggtct^{m}cgttttAA | 3-14-2 |
| 382 | CCCCAGGGGTCTCGTTTT | 382_1 | CCCCaggggtct^{m}cgttttAA | 4-14-2 |
| | AA | | | |
| 383 | TGCACATTTGATAAATTTT | 383_1 | TGCacatttgataaattTTG | 3-14-3 |
| | G | | | |
| 384 | GTGCACATTTGATAAATTT | 384_1 | GTgcacatttgataaaTTTT | 2-14-4 |
| | T | | | |
| 385 | GTGCACATTTGATAAATTT | 385_1 | GTGcacatttgataaaTTT | 3-13-3 |
| 386 | GTGCACATTTGATAAATT | 386_1 | GTGCacatttgataaATT | 4-11-3 |
| 386 | GTGCACATTTGATAAATT | 386_2 | GTGcacatttgataAATT | 3-11-4 |
| 387 | CGTGCACATTTGATAAATT | 387_1 | CGTGcacatttgataaaTT | 4-13-2 |
| 388 | CGTGCACATTTGATAAAT | 388_1 | CGTGcacatttgatAAAT | 4-10-4 |
| 388 | CGTGCACATTTGATAAAT | 388_2 | CGTGcacatttgataaAT | 4-12-2 |
| 389 | ACGTGCACATTTGATAAAT | 389_1 | ACGTgcacatttgataAAT | 4-12-3 |
| 390 | CACGTGCACATTTGATAAA | 390_1 | CA^{m}cgtgcacatttgataAAT | 2-15-3 |
| | T | | | |
| 391 | CGTGCACATTTGATAAA | 391_1 | CGTGcacatttgaTAAA | 4-9-4 |
| 392 | ACGTGCACATTTGATAAA | 392_1 | ACGTgcacatttgaTAAA | 4-10-4 |
| 393 | CACGTGCACATTTGATAAA | 393_1 | CACGtgcacatttgaTAAA | 4-11-4 |
| 394 | ACACGTGCACATTTGATAA | 394_1 | ACACgtgcacatttgataAA | 4-14-2 |
| | A | | | |
| 394 | ACACGTGCACATTTGATAA | 394_2 | ACA^{m}cgtgcacatttgataAA | 3-15-2 |
| | A | | | |
| 395 | ACGTGCACATTTGATAA | 395_1 | ACGTgcacatttgaTAA | 4-10-3 |
| 396 | CACGTGCACATTTGATAA | 396_1 | CACGtgcacatttgaTAA | 4-11-3 |
| 397 | CACACGTGCACATTTGATA | 397_1 | Caca^{m}cgtgcacatttgaTAA | 1-16-3 |
| | A | | | |
| 398 | CACACGTGCACATTTGATA | 398_1 | CAca^{m}cgtgcacatttgaTA | 2-15-2 |
| 398 | CACACGTGCACATTTGATA | 398_2 | Caca^{m}cgtgcacatttgaTA | 1-16-2 |
| 399 | CACACGTGCACATTTGAT | 399_1 | CAca^{m}cgtgcacatttgAT | 2-14-2 |
| 400 | CGGTGGACACAGCGTG | 400_1 | Cggtggacacag^{m}cgTG | 1-13-2 |
| 401 | GAGGACGTCAAGCCG | 401_1 | Gagga^{m}cgtcaagcCG | 1-12-2 |
| 402 | GGAGGACGTCAAGCCG | 402_1 | Ggagga^{m}cgtcaagcCG | 1-13-2 |
| 403 | CGGAGGACGTCAAGCC | 403_1 | Cggagga^{m}cgtcaagCC | 1-13-2 |
| 404 | CCGGAGGACGTCAAGC | 404_1 | C^{m}cggagga^{m}cgtcAAGC | 1-11-4 |
| 404 | CCGGAGGACGTCAAGC | 404_2 | C^{m}cggagga^{m}cgtcaaGC | 1-13-2 |
| 405 | AGAGCGGGATCCTCCA | 405_1 | Agag^{m}cgggatcctcCA | 1-13-2 |
| 406 | CACAGAGCGGGATCCTC | 406_1 | Cacagag^{m}cgggatccTC | 1-14-2 |
| 407 | GCACAGAGCGGGATCC | 407_1 | Gcacagag^{m}cgggatCC | 1-13-2 |
| 408 | AGGGCACAGAGCGGGAT | 408_1 | Agggcacagag^{m}cgggAT | 1-14-2 |
| 409 | CTCTGTGGTCATAGAAAA | 409_1 | CTCTgtggtcatagAAAA | 4-10-4 |
| 409 | CTCTGTGGTCATAGAAAA | 409_2 | CTCTgtggtcatagaAAA | 4-11-3 |
| 409 | CTCTGTGGTCATAGAAAA | 409_3 | CTCTgtggtcatagaaAA | 4-12-2 |
| 410 | GCTCTGTGGTCATAGAAAA | 410_1 | GCtctgtggtcatagAAAA | 2-13-4 |
| 410 | GCTCTGTGGTCATAGAAAA | 410_2 | GCtctgtggtcatagaAAA | 2-14-3 |
| 410 | GCTCTGTGGTCATAGAAAA | 410_3 | GCtctgtggtcatagaaAA | 2-15-2 |
| 411 | AGCTCTGTGGTCATAGAAA | 411 1 | AGCtctgtggtcatagaaAA | 3-15-2 |
| | A | | | |
| 411 | AGCTCTGTGGTCATAGAAA | 411_2 | AGctctgtggtcatagaAAA | 2-15-3 |
| | A | | | |
| 411 | AGCTCTGTGGTCATAGAAA | 411_3 | AgctctgtggtcatagAAAA | 1-15-4 |
| | A | | | |
| 412 | GCTCTGTGGTCATAGAAA | 412_1 | GCtctgtggtcatagaAA | 2-14-2 |
| 413 | AGCTCTGTGGTCATAGAAA | 413_1 | AgctctgtggtcataGAAA | 1-14-4 |
| 413 | AGCTCTGTGGTCATAGAAA | 413_2 | AgctctgtggtcatagAAA | 1-15-3 |
| 414 | GAGCTCTGTGGTCATAGA | 414_1 | GAgctctgtggtcatagaAA | 2-16-2 |
| | AA | | | |
| 414 | GAGCTCTGTGGTCATAGA | 414_2 | GagctctgtggtcatagAAA | 1-16-3 |
| | AA | | | |
| 414 | GAGCTCTGTGGTCATAGA | 414_3 | GagctctgtggtcatagaAA | 1-17-2 |
| | AA | | | |
| 415 | GCTCTGTGGTCATAGAA | 415_1 | GCtctgtggtcataGAA | 2-12-3 |
| 415 | GCTCTGTGGTCATAGAA | 415_2 | GCtctgtggtcatagAA | 2-13-2 |
| 416 | AGCTCTGTGGTCATAGAA | 416_1 | AGCtctgtggtcatagAA | 3-13-2 |
| 416 | AGCTCTGTGGTCATAGAA | 416_2 | AgctctgtggtcataGAA | 1-14-3 |
| 416 | AGCTCTGTGGTCATAGAA | 416_3 | AgctctgtggtcatagAA | 1-15-2 |
| 417 | GAGCTCTGTGGTCATAGA | 417_1 | GAgctctgtggtcatagAA | 2-15-2 |
| | A | | | |
| 417 | GAGCTCTGTGGTCATAGA | 417_2 | GagctctgtggtcataGAA | 1-15-3 |
| | A | | | |
| 417 | GAGCTCTGTGGTCATAGA | 417_3 | GagctctgtggtcatagAA | 1-16-2 |
| | A | | | |
| 418 | GGAGCTCTGTGGTCATAG | 418_1 | GgagctctgtggtcatagAA | 1-17-2 |
| | AA | | | |
| 419 | GAGCTCTGTGGTCATAGA | 419_1 | GagctctgtggtcataGA | 1-15-2 |
| 420 | GGAGCTCTGTGGTCATA | 420_1 | GgagctctgtggtcATA | 1-13-3 |
| 421 | CGGAGCTCTGTGGTCATA | 421_1 | CggagctctgtggtcATA | 1-14-3 |
| 422 | CGGAGCTCTGTGGTCAT | 422_1 | CggagctctgtggtcAT | 1-14-2 |
| 423 | CAGGTGAAGGAAGGCCAG | 423_1 | CaggtgaaggaaggcCAG | 1-14-3 |
| 423 | CAGGTGAAGGAAGGCCAG | 423_2 | CaggtgaaggaaggccAG | 1-15-2 |
| 424 | CCAGGTGAAGGAAGGCCA | 424_1 | CCAggtgaaggaaggCCA | 3-12-3 |
| 425 | CCCAGGTGAAGGAAGGCC | 425_1 | CCCaggtgaaggaaggCC | 3-13-3 |
| | A | | A | |
| 426 | CCCAGGTGAAGGAAGGCC | 426_1 | CCCaggtgaaggaaggCC | 3-13-2 |
| 427 | CCCCAGGTGAAGGAAGGC | 427_1 | CCCCaggtgaaggaagGC | 4-12-2 |
| 428 | CTGTGCTGAAGATGGGC | 428_1 | CtgtgctgaagatggGC | 1-14-2 |
| 429 | CCTGTGCTGAAGATGGG | 429_1 | CctgtgctgaagatGGG | 1-13-3 |
| 429 | CCTGTGCTGAAGATGGG | 429_2 | CctgtgctgaagatgGG | 1-14-2 |
| 430 | ATTGCGGCACGGGCTG | 430_1 | Attg^{m}cggca^{m}cgggcTG | 1-13-2 |
| 431 | ATTTTACTTATCCCCAGCC | 431_1 | AttttacttatccccagCC | 1-16-2 |
| 432 | CATTTTACTTATCCCCAGC | 432_1 | CAttttacttatccccagCC | 2-16-2 |
| | C | | | |
| 433 | TTTTACTTATCCCCAGC | 433_1 | TtttacttatccccAGC | 1-13-3 |
| 433 | TTTTACTTATCCCCAGC | 433_2 | TtttacttatccccaGC | 1-14-2 |
| 434 | ATTTTACTTATCCCCAGC | 434_1 | AttttacttatccccAGC | 1-14-3 |
| 434 | ATTTTACTTATCCCCAGC | 434_2 | AttttacttatccccaGC | 1-15-2 |
| 435 | CATTTTACTTATCCCCAG | 435_1 | CAttttacttatccccAG | 2-14-2 |
| 435 | CATTTTACTTATCCCCAG | 435_2 | CattttacttatccccAG | 1-15-2 |
| 436 | CCATTTTACTTATCCCCAG | 436_1 | CcattttacttatccccAG | 1-16-2 |
| 437 | CCATTTTACTTATCCCCA | 437_1 | CcattttacttatcccCA | 1-15-2 |
| 438 | CCATTTTACTTATCCCC | 438_1 | CCattttacttatccCC | 2-13-2 |
| 438 | CCATTTTACTTATCCCC | 438_2 | CcattttacttatccCC | 1-14-2 |
| 439 | CTCTGTAGTTTGTTCTC | 439_1 | CtctgtagtttgttcTC | 1-14-2 |
| 440 | ACTGCACCGGGACACAG | 440_1 | Actgcac^{m}cgggacacAG | 1-14-2 |
| 441 | GCCCGCTAGAAGCCCC | 441_1 | Gcc^{m}cgctagaagCCCC | 1-11-4 |
| 442 | ACCTACCTCATCACCAC | 442_1 | AcctacctcatcaccAC | 1-14-2 |
| 443 | ACACCTACCTCATCACC | 443_1 | AcacctacctcatcaCC | 1-14-2 |
| 444 | AACACCTACCTCATCACC | 444_1 | AacacctacctcatcaCC | 1-15-2 |
| 445 | AAACACCTACCTCATCACC | 445_1 | AaacacctacctcatcaCC | 1-16-2 |
| 446 | AAACACCTACCTCATCAC | 446_1 | AaacacctacctcatCAC | 1-14-3 |
| 447 | CAAACACCTACCTCATCAC | 447_1 | CAAAcacctacctcatcAC | 4-13-2 |
| 447 | CAAACACCTACCTCATCAC | 447_2 | CaaacacctacctcatCAC | 1-15-3 |
| 447 | CAAACACCTACCTCATCAC | 447_3 | CAaacacctacctcatcAC | 2-15-2 |
| 448 | GCAAACACCTACCTCATCA | 448_1 | GcaaacacctacctcatcAC | 1-17-2 |
| | C | | | |
| 449 | CAAACACCTACCTCATCA | 449_1 | CaaacacctacctcaTCA | 1-14-3 |
| 449 | CAAACACCTACCTCATCA | 449_2 | CaaacacctacctcatCA | 1-15-2 |
| 450 | GCAAACACCTACCTCATCA | 450_1 | GcaaacacctacctcatCA | 1-16-2 |
| 451 | CAAACACCTACCTCATC | 451_1 | CAAAcacctacctcATC | 4-10-3 |
| 451 | CAAACACCTACCTCATC | 451_2 | CAaacacctacctcATC | 2-12-3 |
| 452 | GCAAACACCTACCTCATC | 452_1 | GCaaacacctacctcaTC | 2-14-2 |
| 452 | GCAAACACCTACCTCATC | 452_2 | GcaaacacctacctcATC | 1-14-3 |
| 452 | GCAAACACCTACCTCATC | 452_3 | GcaaacacctacctcaTC | 1-15-2 |
| 453 | GCAAACACCTACCTCAT | 453_1 | GCaaacacctacctcAT | 2-13-2 |
| 454 | CCTACATGGGGGCTTG | 454_1 | CctacatgggggcTTG | 1-12-3 |
| 454 | CCTACATGGGGGCTTG | 454_2 | CctacatgggggctTG | 1-13-2 |
| 455 | GCCTACATGGGGGCTT | 455_1 | GcctacatgggggcTT | 1-13-2 |
| 456 | TTGGGAGAGAACCTTCAG | 456_1 | TTgggagagaaccttcAG | 2-14-2 |
| 457 | ATTGGGAGAGAACCTTCA | 457_1 | AttgggagagaaccttcAG | 1-16-2 |
| | G | | | |
| 458 | AATTGGGAGAGAACCTTCA | 458_1 | AAttgggagagaaccttcAG | 2-16-2 |
| | G | | | |
| 459 | ATTGGGAGAGAACCTTCA | 459_1 | ATtgggagagaaccttCA | 2-14-2 |
| 460 | AATTGGGAGAGAACCTTCA | 460_1 | AAttgggagagaacctTCA | 2-14-3 |
| 461 | CAATTGGGAGAGAACCTT | 461_1 | CaattgggagagaaccttCA | 1-17-2 |
| | CA | | | |
| 462 | CAATTGGGAGAGAACCTT | 462_1 | CAATtgggagagaacctTC | 4-13-2 |
| | C | | | |
| 462 | CAATTGGGAGAGAACCTT | 462_2 | CaattgggagagaaccTTC | 1-15-3 |
| | C | | | |
| 463 | CAATTGGGAGAGAACCTT | 463_1 | CAattgggagagaacCTT | 2-13-3 |
| 464 | CAATTGGGAGAGAACCT | 464_1 | CAATtgggagagaacCT | 4-11-2 |
| 465 | AAAGCATCTGTGGGCATG | 465_1 | AaagcatctgtgggCATG | 1-13-4 |
| 466 | CCAAAGCATCTGTGGGCA | 466_1 | CcaaagcatctgtgggCA | 1-15-2 |
| 467 | CCATCACTCCAAAGCAT | 467_1 | CCatcactccaaagcAT | 2-13-2 |
| 468 | AAAGGAGAGTCGTGCCTG | 468_1 | Aaaggagagt^{m}cgtgccTG | 1-15-2 |
| 469 | AAAGGAGAGTCGTGCCT | 469_1 | Aaaggagagt^{m}cgtgCCT | 1-13-3 |
| 469 | AAAGGAGAGTCGTGCCT | 469_2 | AAAggagagt^{m}cgtgcCT | 3-12-2 |
| 469 | AAAGGAGAGTCGTGCCT | 469_3 | AAaggagagt^{m}cgtgcCT | 2-13-2 |
| 470 | AAAGGAGAGTCGTGCC | 470_1 | AAAggagagt^{m}cgtGCC | 3-10-3 |
| 470 | AAAGGAGAGTCGTGCC | 470_2 | AAaggagagt^{m}cgtGCC | 2-11-3 |
| 470 | AAAGGAGAGTCGTGCC | 470_3 | AAAggagagt^{m}cgtgCC | 3-11-2 |
| 471 | GAAAGGAGAGTCGTGCC | 471_1 | Gaaaggagagt^{m}cgtgCC | 1-14-2 |
| 472 | TGGAAAGGAGAGTCGTGC | 472_1 | Tggaaaggagagt^{m}cgtgCC | 1-16-2 |
| | C | | | |
| 473 | CTGGAAAGGAGAGTCGTG | 473_1 | Ctggaaaggagagt^{m}cgtgC | 1-17-2 |
| | CC | | C | |
| 474 | GAAAGGAGAGTCGTGC | 474_1 | GaaaggagagtcGTGC | 1-11-4 |
| 474 | GAAAGGAGAGTCGTGC | 474_2 | GAAaggagagt^{m}cgtGC | 3-11-2 |
| 475 | GGAAAGGAGAGTCGTGC | 475_1 | Ggaaaggagagt^{m}cgtGC | 1-14-2 |
| 476 | TGGAAAGGAGAGTCGTGC | 476_1 | TGgaaaggagagt^{m}cgtGC | 2-14-2 |
| 476 | TGGAAAGGAGAGTCGTGC | 476_2 | Tggaaaggagagt^{m}cgtGC | 1-15-2 |
| 477 | CTGGAAAGGAGAGTCGTG | 477_1 | Ctggaaaggagagt^{m}cgtGC | 1-16-2 |
| | C | | | |
| 478 | CCTGGAAAGGAGAGTCGT | 478_1 | CCtggaaaggagagt^{m}cgtG | 2-16-2 |
| | GC | | C | |
| 479 | TGGAAAGGAGAGTCGTG | 479_1 | TggaaaggagagtCGTG | 1-12-4 |
| 479 | TGGAAAGGAGAGTCGTG | 479_2 | TGgaaaggagagtcGTG | 2-12-3 |
| 480 | CTGGAAAGGAGAGTCGTG | 480_1 | CtggaaaggagagtCGTG | 1-13-4 |
| 480 | CTGGAAAGGAGAGTCGTG | 480_2 | CTggaaaggagagt^{m}cgTG | 2-14-2 |
| 480 | CTGGAAAGGAGAGTCGTG | 480_3 | CtggaaaggagagtcGTG | 1-14-3 |
| 481 | CCTGGAAAGGAGAGTCGT | 481_1 | Cctggaaaggagagt^{m}cgTG | 1-16-2 |
| | G | | | |
| 482 | CTGGAAAGGAGAGTCGT | 482_1 | CTggaaaggagagTCGT | 2-11-4 |
| 482 | CTGGAAAGGAGAGTCGT | 482_2 | CTggaaaggagagtCGT | 2-12-3 |
| 482 | CTGGAAAGGAGAGTCGT | 482_3 | CtggaaaggagagtCGT | 1-13-3 |
| 483 | CCTGGAAAGGAGAGTCGT | 483_1 | CctggaaaggagagtcGT | 1-15-2 |
| 484 | CCTGGAAAGGAGAGTCG | 484_1 | CCtggaaaggagagTCG | 2-12-3 |
| 484 | CCTGGAAAGGAGAGTCG | 484_2 | CCtggaaaggagagtCG | 2-13-2 |
| 484 | CCTGGAAAGGAGAGTCG | 484_3 | CctggaaaggagagTCG | 1-13-3 |
| 485 | CTACAACAAAGCCCGAGG | 485_1 | Ctacaacaaagcc^{m}cgAGG | 1-14-3 |
| 485 | CTACAACAAAGCCCGAGG | 485_2 | Ctacaacaaagcc^{m}cgaGG | 1-15-2 |
| 486 | TTCTACAACAAAGCCCGAG | 486_1 | Ttctacaacaaagcc^{m}cgaG | 1-17-2 |
| | G | | G | |
| 487 | CTACAACAAAGCCCGAG | 487_1 | CtacaacaaagcccGAG | 1-13-3 |
| 488 | TCTACAACAAAGCCCGAG | 488_1 | TCtacaacaaagcc^{m}cgAG | 2-14-2 |
| 489 | TTCTACAACAAAGCCCGAG | 489_1 | TTCtacaacaaagcc^{m}cgAG | 3-14-2 |
| 489 | TTCTACAACAAAGCCCGAG | 489_2 | TTctacaacaaagcc^{m}cgAG | 2-15-2 |
| 489 | TTCTACAACAAAGCCCGAG | 489_3 | Ttctacaacaaagcc^{m}cgAG | 1-16-2 |
| 490 | TTTCTACAACAAAGCCCGA | 490_1 | TTtctacaacaaagcc^{m}cgAG | 2-16-2 |
| | G | | | |
| 490 | TTTCTACAACAAAGCCCGA | 490_2 | Tttctacaacaaagcc^{m}cgAG | 1-17-2 |
| | G | | | |
| 491 | TCTACAACAAAGCCCGA | 491_1 | TctacaacaaagccCGA | 1-13-3 |
| 492 | TTTCTACAACAAAGCCCGA | 492_1 | TTtctacaacaaagccCGA | 2-14-3 |
| 492 | TTTCTACAACAAAGCCCGA | 492_2 | TttctacaacaaagcccGA | 1-16-2 |
| 493 | GTTTCTACAACAAAGCCCG | 493_1 | GtttctacaacaaagcccGA | 1-17-2 |
| | A | | | |
| 494 | TCTACAACAAAGCCCG | 494_1 | TCtacaacaaagCCCG | 2-10-4 |
| 494 | TCTACAACAAAGCCCG | 494_2 | TctacaacaaagCCCG | 1-11-4 |
| 495 | GTTTCTACAACAAAGCCCG | 495_1 | GtttctacaacaaagcCCG | 1-15-3 |
| 495 | GTTTCTACAACAAAGCCCG | 495_2 | GtttctacaacaaagccCG | 1-16-2 |
| 496 | GTTTCTACAACAAAGCCC | 496_1 | GTTtctacaacaaagcCC | 3-13-2 |
| 496 | GTTTCTACAACAAAGCCC | 496_2 | GtttctacaacaaagcCC | 1-15-2 |
| 497 | TGTTTCTACAACAAAGCCC | 497_1 | TGtttctacaacaaagcCC | 2-15-2 |
| 497 | TGTTTCTACAACAAAGCCC | 497_2 | TgtttctacaacaaagcCC | 1-16-2 |
| 498 | TTGTTTCTACAACAAAGCC | 498_1 | TTgtttctacaacaaagcCC | 2-16-2 |
| | C | | | |
| 498 | TTGTTTCTACAACAAAGCC | 498_2 | TtgtttctacaacaaagcCC | 1-17-2 |
| | C | | | |
| 499 | TGTTTCTACAACAAAGCC | 499_1 | TGtttctacaacaaaGCC | 2-13-3 |
| 499 | TGTTTCTACAACAAAGCC | 499_2 | TgtttctacaacaaaGCC | 1-14-3 |
| 499 | TGTTTCTACAACAAAGCC | 499_3 | TGtttctacaacaaagCC | 2-14-2 |
| 500 | TTGTTTCTACAACAAAGCC | 500_1 | TtgtttctacaacaaAGCC | 1-14-4 |
| 500 | TTGTTTCTACAACAAAGCC | 500_2 | TtgtttctacaacaaaGCC | 1-15-3 |
| 500 | TTGTTTCTACAACAAAGCC | 500_3 | TTgtttctacaacaaagCC | 2-15-2 |
| 501 | ATTGTTTCTACAACAAAGC | 501_1 | AttgtttctacaacaaaGCC | 1-16-3 |
| | C | | | |
| 501 | ATTGTTTCTACAACAAAGC | 501_2 | ATtgtttctacaacaaagCC | 2-16-2 |
| | C | | | |
| 501 | ATTGTTTCTACAACAAAGC | 501_3 | AttgtttctacaacaaagCC | 1-17-2 |
| | C | | | |
| 502 | TTGTTTCTACAACAAAGC | 502_1 | TTGTttctacaacaaAGC | 4-11-3 |
| 502 | TTGTTTCTACAACAAAGC | 502_2 | TTGTttctacaacaaaGC | 4-12-2 |
| 503 | ATTGTTTCTACAACAAAGC | 503_1 | ATTGtttctacaacaaAGC | 4-12-3 |
| 503 | ATTGTTTCTACAACAAAGC | 503_2 | ATTgtttctacaacaaaGC | 3-14-2 |
| 504 | CATTGTTTCTACAACAAAG | 504_1 | CAttgtttctacaacaAAGC | 2-14-4 |
| | C | | | |
| 504 | CATTGTTTCTACAACAAAG | 504_2 | CattgtttctacaacaaAGC | 1-16-3 |
| | C | | | |
| 505 | ATTGTTTCTACAACAAAG | 505_1 | ATTGtttctacaacAAAG | 4-10-4 |
| 506 | CATTGTTTCTACAACAAAG | 506_1 | CATTgtttctacaacAAAG | 4-11-4 |
| 507 | CCATTGTTTCTACAACAAA | 507_1 | CCAttgtttctacaacaAAG | 3-14-3 |
| | G | | | |
| 507 | CCATTGTTTCTACAACAAA | 507_2 | CCattgtttctacaacaaAG | 2-16-2 |
| | G | | | |
| 508 | CCATTGTTTCTACAACAAA | 508_1 | CCAttgtttctacaaCAAA | 3-12-4 |
| 509 | GCCATTGTTTCTACAACAA | 509_1 | GCcattgtttctacaaCAAA | 2-14-4 |
| | A | | | |
| 509 | GCCATTGTTTCTACAACAA | 509_2 | GCcattgtttctacaacaAA | 2-16-2 |
| | A | | | |
| 509 | GCCATTGTTTCTACAACAA | 509_3 | GccattgtttctacaaCAAA | 1-15-4 |
| | A | | | |
| 510 | CCATTGTTTCTACAACAA | 510_1 | CCAttgtttctacaACAA | 3-11-4 |
| 511 | GCCATTGTTTCTACAACAA | 511_1 | GCcattgtttctacaaCAA | 2-14-3 |
| 511 | GCCATTGTTTCTACAACAA | 511_2 | GccattgtttctacaACAA | 1-14-4 |
| 511 | GCCATTGTTTCTACAACAA | 511_3 | GccattgtttctacaaCAA | 1-15-3 |
| 512 | GGCCATTGTTTCTACAACA | 512_1 | GGccattgtttctacaacAA | 2-16-2 |
| | A | | | |
| 513 | GCCATTGTTTCTACAACA | 513_1 | GCcattgtttctacaaCA | 2-14-2 |
| 513 | GCCATTGTTTCTACAACA | 513_2 | GccattgtttctacaACA | 1-14-3 |
| 514 | GGCCATTGTTTCTACAACA | 514_1 | GgccattgtttctacaaCA | 1-16-2 |
| 515 | GGCCATTGTTTCTACAAC | 515_1 | GgccattgtttctaCAAC | 1-13-4 |
| 515 | GGCCATTGTTTCTACAAC | 515_2 | GGccattgtttctacaAC | 2-14-2 |
| 516 | TTTCAGATGCCAAGACACA | 516_1 | TttcagatgccaagacaCA | 1-16-2 |
| 517 | ATTTCAGATGCCAAGACAC | 517_1 | AtttcagatgccaagacACA | 1-16-3 |
| | A | | | |
| 517 | ATTTCAGATGCCAAGACAC | 517_2 | AtttcagatgccaagacaCA | 1-17-2 |
| | A | | | |
| 518 | ATTTCAGATGCCAAGACAC | 518_1 | ATttcagatgccaagaCAC | 2-14-3 |
| 519 | CATTTCAGATGCCAAGACA | 519_1 | CatttcagatgccaagaCAC | 1-16-3 |
| | C | | | |
| 519 | CATTTCAGATGCCAAGACA | 519_2 | CAtttcagatgccaagacAC | 2-16-2 |
| | C | | | |
| 519 | CATTTCAGATGCCAAGACA | 519_3 | CatttcagatgccaagacAC | 1-17-2 |
| | C | | | |
| 520 | ATTTCAGATGCCAAGACA | 520_1 | ATttcagatgccaagaCA | 2-14-2 |
| 521 | CATTTCAGATGCCAAGACA | 521_1 | CAtttcagatgccaagACA | 2-14-3 |
| 522 | GCATTTCAGATGCCAAGAC | 522_1 | GcatttcagatgccaagAC | 1-16-2 |
| 523 | GTAGCCTGCATTTCAGAT | 523_1 | GtagcctgcatttcagAT | 1-15-2 |
| 524 | TACCTGCGGTAGTTCT | 524_1 | Tacctg^{m}cggtagtTCT | 1-12-3 |
| 524 | TACCTGCGGTAGTTCT | 524_2 | Tacctg^{m}cggtagttCT | 1-13-2 |
| 525 | CTACCTGCGGTAGTTCT | 525_1 | Ctacctg^{m}cggtagtTCT | 1-13-3 |
| 525 | CTACCTGCGGTAGTTCT | 525_2 | Ctacctg^{m}cggtagttCT | 1-14-2 |
| 526 | CTACCTGCGGTAGTTC | 526_1 | CTacctg^{m}cggtagtTC | 2-12-2 |
| 526 | CTACCTGCGGTAGTTC | 526_2 | Ctacctg^{m}cggtagtTC | 1-13-2 |
| 527 | CCTACCTGCGGTAGTTC | 527_1 | Cctacctg^{m}cggtagtTC | 1-14-2 |
| 528 | CTACCTGCGGTAGTT | 528_1 | CTACctg^{m}cggtagTT | 4-9-2 |
| 528 | CTACCTGCGGTAGTT | 528_2 | CTAcctg^{m}cggtagTT | 3-10-2 |
| 528 | CTACCTGCGGTAGTT | 528_3 | CTacctg^{m}cggtagTT | 2-11-2 |
| 529 | CCTACCTGCGGTAGTT | 529_1 | Cctacctg^{m}cggtagTT | 1-13-2 |
| 530 | GCCTACCTGCGGTAGTT | 530_1 | Gcctacctg^{m}cggtagTT | 1-14-2 |
| 531 | GCCTACCTGCGGTAG | 531_1 | Gcctacctg^{m}cggTAG | 1-11-3 |
| 531 | GCCTACCTGCGGTAG | 531_2 | Gcctacctg^{m}cggtAG | 1-12-2 |
| 532 | CGCCTACCTGCGGTAG | 532_1 | Cgcctacctg^{m}cggtAG | 1-13-2 |
| 533 | TTTTGGAGAAGCCTGGGG | 533_1 | TtttggagaagcctggGG | 1-15-2 |
| 534 | GTTTTGGAGAAGCCTGGG | 534_1 | GttttggagaagcctgGG | 1-15-2 |
| 535 | CCGTTTTGGAGAAGCCTG | 535_1 | C^{m}cgttttggagaagcctgGG | 1-17-2 |
| | GG | | | |
| 536 | CGTTTTGGAGAAGCCTGG | 536_1 | CgttttggagaagccTGG | 1-14-3 |
| 536 | CGTTTTGGAGAAGCCTGG | 536_2 | CGttttggagaagcctGG | 2-14-2 |
| 537 | CCCGTTTTGGAGAAGCCT | 537_1 | Cc^{m}cgttttggagaagccTGG | 1-16-3 |
| | GG | | | |
| 538 | CGTTTTGGAGAAGCCTG | 538_1 | CGttttggagaagcCTG | 2-12-3 |
| 538 | CGTTTTGGAGAAGCCTG | 538_2 | CgttttggagaagcCTG | 1-13-3 |
| 538 | CGTTTTGGAGAAGCCTG | 538_3 | CGttttggagaagccTG | 2-13-2 |
| 539 | CCGTTTTGGAGAAGCCTG | 539_1 | C^{m}cgttttggagaagccTG | 1-15-2 |
| 540 | CCCGTTTTGGAGAAGCCT | 540_1 | Cc^{m}cgttttggagaagccTG | 1-16-2 |
| | G | | | |
| 541 | GCCCGTTTTGGAGAAGCC | 541 1 | GCc^{m}cgttttggagaagccTG | 2-16-2 |
| | TG | | | |
| 542 | CGTTTTGGAGAAGCCT | 542_1 | CGttttggagaagCCT | 2-11-3 |
| 542 | CGTTTTGGAGAAGCCT | 542_2 | CGTtttggagaagcCT | 3-11-2 |
| 542 | CGTTTTGGAGAAGCCT | 542_3 | CGttttggagaagcCT | 2-12-2 |
| 543 | CCGTTTTGGAGAAGCCT | 543_1 | C^{m}cgttttggagaagCCT | 1-13-3 |
| 543 | CCGTTTTGGAGAAGCCT | 543_2 | C^{m}cgttttggagaagcCT | 1-14-2 |
| 544 | CCCGTTTTGGAGAAGCCT | 544_1 | Cc^{m}cgttttggagaagCCT | 1-14-3 |
| 545 | GCCCGTTTTGGAGAAGCC | 545_1 | GCC^{m}cgttttggagaagcCT | 3-14-2 |
| | T | | | |
| 546 | CCCGTTTTGGAGAAGCC | 546_1 | Cc^{m}cgttttggagaagCC | 1-14-2 |
| 547 | GCCCGTTTTGGAGAAGCC | 547_1 | GCc^{m}cgttttggagaagCC | 2-14-2 |
| 548 | AGCCCGTTTTGGAGAAGC | 548_1 | AGCc^{m}cgttttggagaagCC | 3-14-2 |
| | C | | | |
| 549 | GCCCGTTTTGGAGAAGC | 549_1 | Gcc^{m}cgttttggagaaGC | 1-14-2 |
| 550 | AGCCCGTTTTGGAGAAGC | 550_1 | AGCc^{m}cgttttggagaaGC | 3-13-2 |
| 551 | CAGCCCGTTTTGGAGAAG | 551 1 | CAGCc^{m}cgttttggagaaGC | 4-13-2 |
| | C | | | |
| 552 | AGCCCGTTTTGGAGAAG | 552_1 | AGcc^{m}cgttttggagaAG | 2-13-2 |
| 552 | AGCCCGTTTTGGAGAAG | 552_2 | Agcc^{m}cgttttggagAAG | 1-13-3 |
| 552 | AGCCCGTTTTGGAGAAG | 552_3 | Agcc^{m}cgttttggagaAG | 1-14-2 |
| 553 | CAGCCCGTTTTGGAGAAG | 553_1 | Cagcc^{m}cgttttggagAAG | 1-14-3 |
| 553 | CAGCCCGTTTTGGAGAAG | 553_2 | Cagcc^{m}cgttttggagaAG | 1-15-2 |
| 554 | CAGCCCGTTTTGGAGAA | 554_1 | Cagcc^{m}cgttttggagAA | 1-14-2 |
| 555 | CCCCAGCCCGTTTTGGAG | 555_1 | CCCCagcc^{m}cgttttggagA | 4-14-2 |
| | AA | | A | |
| 556 | TTCAGGGCACCAGATTC | 556_1 | TTCagggcaccagatTC | 3-12-2 |
| 556 | TTCAGGGCACCAGATTC | 556_2 | TtcagggcaccagatTC | 1-14-2 |
| 557 | TTTCAGGGCACCAGATTC | 557_1 | TttcagggcaccagatTC | 1-15-2 |
| 558 | CTTTCAGGGCACCAGATT | 558_1 | CtttcagggcaccagATT | 1-14-3 |
| 558 | CTTTCAGGGCACCAGATT | 558_2 | CtttcagggcaccagaTT | 1-15-2 |
| 559 | GTGCCGCTTAACAAAC | 559_1 | GTGC^{m}cgcttaacaAAC | 4-9-3 |
| 560 | AGTGCCGCTTAACAAAC | 560_1 | AGTGc^{m}cgcttaacaaAC | 4-11-2 |
| 561 | TGAGTGCCGCTTAACAAAC | 561_1 | TGagtgc^{m}cgcttaacAAAC | 2-13-4 |
| 561 | TGAGTGCCGCTTAACAAAC | 561_2 | TGagtgc^{m}cgcttaacaaAC | 2-15-2 |
| 562 | AGTGCCGCTTAACAAA | 562_1 | AGTGc^{m}cgcttaaCAAA | 4-8-4 |
| 563 | TGAGTGCCGCTTAACAAA | 563_1 | Tgagtgc^{m}cgcttaaCAAA | 1-13-4 |
| 564 | TGAGTGCCGCTTAACAA | 564_1 | Tgagtgc^{m}cgcttaaCAA | 1-13-3 |
| 565 | TGAGTGCCGCTTAACA | 565_1 | TGAgtgc^{m}cgcttaaCA | 3-11-2 |
| 565 | TGAGTGCCGCTTAACA | 565_2 | Tgagtgc^{m}cgcttaACA | 1-12-3 |
| 566 | ACAGATGGCGTGTGCATG | 566_1 | Acagatgg^{m}cgtgtgcATG | 1-14-3 |
| 567 | TACACAGATGGCGTGTG | 567_1 | Tacacagatgg^{m}cgTGTG | 1-12-4 |
| 568 | TTACACAGATGGCGTGTG | 568_1 | TTAcacagatgg^{m}cgtgTG | 3-13-2 |
| 568 | TTACACAGATGGCGTGTG | 568_2 | Ttacacagatgg^{m}cgtgTG | 1-15-2 |
| 569 | TTACACAGATGGCGTGT | 569_1 | TTacacagatgg^{m}cgTGT | 2-12-3 |
| 570 | GTTACACAGATGGCGTGT | 570_1 | Gttacacagatgg^{m}cgTGT | 1-14-3 |
| 571 | ATGTATTGTGTGTTACATG | 571_1 | AtgtattgtgtgttacatGG | 1-17-2 |
| | G | | | |
| 572 | ATGTATTGTGTGTTACATG | 572_1 | ATgtattgtgtgttaCATG | 2-13-4 |
| 573 | CATGTATTGTGTGTTACAT | 573_1 | CatgtattgtgtgttaCATG | 1-15-4 |
| | G | | | |
| 574 | ATGTATTGTGTGTTACAT | 574_1 | ATGtattgtgtgttACAT | 3-11-4 |
| 575 | ACCCGTGCTGTTTATTTA | 575_1 | ACc^{m}cgtgctgtttattTA | 2-14-2 |
| 575 | ACCCGTGCTGTTTATTTA | 575_2 | Acc^{m}cgtgctgtttattTA | 1-15-2 |
| 576 | ACCCGTGCTGTTTATTT | 576_1 | ACC^{m}cgtgctgtttatTT | 3-12-2 |
| 576 | ACCCGTGCTGTTTATTT | 576_2 | Acc^{m}cgtgctgtttatTT | 1-14-2 |
| 577 | CACCCGTGCTGTTTATTT | 577_1 | CAcc^{m}cgtgctgtttatTT | 2-14-2 |

In the specific compounds tested (see column “Oligonucleotide compound”), capital letters are beta-D-oxy LNA nucleosides, all LNA Cs are beta-D-oxy-LNA 5-methyl cytosine, lower case letters are DNA nucleosides, and a superscript m before a lower case c represent a 5-methyl cytosine DNA nucleoside, otherwise DNA c nucleosides are cytosine nucleosides, and all internucleoside linkages are phosphorothioate internucleoside linkages. The methylation of the cytosine DNA nucleosides of the compounds provided in the table is an optional feature. The cytosine DNA nucleoside might be also unmethylated.

The invention provides antisense oligonucleotides according to the invention, such as antisense oligonucleotides 12-24, such as 12-18 in length, nucleosides in length wherein the antisense oligonucleotide comprises a contiguous nucleotide sequence comprising at least 12, such as at least 14, such as at least 15 contiguous nucleotides present in any one of the sequence motifs listed in Table 2 (see column “Sequence motifs”).

The antisense oligonucleotides provided herein typically comprise or consist of a contiguous nucleotide sequence selected from SEQ ID NO 70-577. For example, the antisense oligonucleotides are LNA gapmers comprising or consisting of a contiguous nucleotide sequence selected from SEQ ID NO 70-577.

The invention provides antisense oligonucleotides selected from the group consisting of the antisense oligonucleotides listed in Table 2 in the column “Oligonucleotide compounds”, wherein a capital letter is a LNA nucleoside, and a lower case letter is a DNA nucleoside. In some embodiments all internucleoside linkages in contiguous nucleoside sequence are phosphorothioate internucleoside linkages. Optionally LNA cytosine may be 5-methyl cytosine. Optionally DNA cytosine may be 5-methyl cytosine.

The invention provides antisense oligonucleotides selected from the group consisting of the antisense oligonucleotides listed in Table 2 in the column “Oligonucleotide compounds”, wherein a capital letter is a beta-D-oxy-LNA nucleoside, and a lower case letter is a DNA nucleoside. In some embodiments all internucleoside linkages in contiguous nucleoside sequence are phosphorothioate internucleoside linkages. Optionally LNA cytosine may be 5-methyl cytosine. Optionally DNA cytosine may be 5-methyl cytosine.

The invention provides antisense oligonucleotides selected from the group consisting of the antisense oligonucleotides listed in Table 2 in the column “Oligonucleotide compounds”, wherein a capital letter is a beta-D-oxy-LNA nucleoside, wherein all LNA cytosinese are 5-methyl cytosine, and a lower case letter is a DNA nucleoside, wherein all internucleoside linkages in contiguous nucleoside sequence are phosphorothioate internucleoside linkages, and optionally DNA cytosine may be 5-methyl cytosine.

Method of Manufacture

In a further aspect, the invention provides methods for manufacturing the oligonucleotides of the invention comprising reacting nucleotide units and thereby forming covalently linked contiguous nucleotide units comprised in the oligonucleotide. Preferably, the method uses phophoramidite chemistry (see for example Caruthers et al, 1987, Methods in Enzymology vol. 154, pages 287-313). In a further embodiment the method further comprises reacting the contiguous nucleotide sequence with a conjugating moiety (ligand) to covalently attach the conjugate moiety to the oligonucleotide. In a further aspect a method is provided for manufacturing the composition of the invention, comprising mixing the oligonucleotide or conjugated oligonucleotide of the invention with a pharmaceutically acceptable diluent, solvent, carrier, salt and/or adjuvant.

Pharmaceutical Composition

In a further aspect, the invention provides pharmaceutical compositions comprising any of the aforementioned oligonucleotides and/or oligonucleotide conjugates or salts thereof and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline.

In some embodiments the oligonucleotide is used in the pharmaceutically acceptable diluent at a concentration of 50-300 μM solution.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin, Organic Process Research & Development 2000, 4, 427-435 or in Ansel, In: Pharmaceutical Dosage Forms and Drug Delivery Systems, 6th ed. (1995), pp. 196 and 1456-1457. For example, the pharmaceutically acceptable salt of the compounds provided herein may be a sodium salt.

Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990). WO 2007/031091 provides further suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations are also provided in WO2007/031091.

Oligonucleotides or oligonucleotide conjugates of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

In some embodiments, the oligonucleotide or oligonucleotide conjugate of the invention is a prodrug. In particular with respect to oligonucleotide conjugates the conjugate moiety is cleaved of the oligonucleotide once the prodrug is delivered to the site of action, e.g. the target cell.

Applications

The oligonucleotides of the invention may be utilized as research reagents for, for example, diagnostics, therapeutics and prophylaxis.

In research, such oligonucleotides may be used to specifically modulate the synthesis of CARD9 protein in cells (e.g. in vitro cell cultures) and experimental animals thereby facilitating functional analysis of the target or an appraisal of its usefulness as a target for therapeutic intervention. Typically the target modulation is achieved by degrading or inhibiting the mRNA producing the protein, thereby prevent protein formation or by degrading or inhibiting a modulator of the gene or mRNA producing the protein.

If employing the oligonucleotide of the invention in research or diagnostics the target nucleic acid may be a cDNA or a synthetic nucleic acid derived from DNA or RNA.

The present invention provides an in vivo or in vitro method for modulating CARD9 expression in a target cell which is expressing CARD9, said method comprising administering an oligonucleotide of the invention in an effective amount to said cell.

In some embodiments, the target cell, is a mammalian cell in particular a human cell. The target cell may be an in vitro cell culture or an in vivo cell forming part of a tissue in a mammal.

In diagnostics the oligonucleotides may be used to detect and quantitate CARD9 expression in cell and tissues by northern blotting, in-situ hybridisation or similar techniques.

For therapeutics, an animal or a human, suspected of having a disease or disorder, which can be treated by modulating the expression of CARD9 The invention provides methods for treating or preventing a disease, comprising administering a therapeutically or prophylactically effective amount of an oligonucleotide, an oligonucleotide conjugate or a pharmaceutical composition of the invention to a subject suffering from or susceptible to the disease.

The invention also relates to an oligonucleotide, a composition or a conjugate as defined herein for use as a medicament.

The oligonucleotide, oligonucleotide conjugate or a pharmaceutical composition according to the invention is typically administered in an effective amount.

The invention also provides for the use of the oligonucleotide or oligonucleotide conjugate of the invention as described for the manufacture of a medicament for the treatment of a disorder as referred to herein, or for a method of the treatment of as a disorder as referred to herein.

The disease or disorder, as referred to herein, is associated with expression of CARD9. In some embodiments disease or disorder may be associated with a mutation in the CARD9 gene. Therefore, in some embodiments, the target nucleic acid is a mutated form of the CARD9 sequence.

The methods of the invention are preferably employed for treatment or prophylaxis against diseases caused by abnormal levels and/or activity of CARD9.

The invention further relates to use of an oligonucleotide, oligonucleotide conjugate or a pharmaceutical composition as defined herein for the manufacture of a medicament for the treatment of abnormal levels and/or activity of CARD9.

In one embodiment, the invention relates to oligonucleotides, oligonucleotide conjugates or pharmaceutical compositions for use in the treatment of diseases or disorders selected from inflammatory bowel disease, pancreatitis, IgA nephropathy, primary sclerosing cholangitis, cardiovascular disease, cancer and diabetes.

In some embodiments, the disease is Inflammatory bowel disease. For example, the inflammatory bowel disease is Crohn's disease. Alternatively, the inflammatory bowel disease is ulcerative colitis.

In some embodiments, the disease is diabetes such as type 2 diabetes.

In some embodiments, the disease is pancreatitis such as acute pancreatitis.

Administration

The oligonucleotides or pharmaceutical compositions of the present invention may be administered topical or enteral or parenteral (such as, intravenous, subcutaneous, intramuscular, intracerebral, intracerebroventricular or intrathecal).

In a preferred embodiment the oligonucleotide or pharmaceutical compositions of the present invention are administered by a parenteral route including intravenous, intraarterial, subcutaneous, intraperitoneal or intramuscular injection or infusion, intrathecal or intracranial, e.g. intracerebral or intraventricular, intravitreal administration. In one embodiment the active oligonucleotide or oligonucleotide conjugate is administered intravenously. In another embodiment the active oligonucleotide or oligonucleotide conjugate is administered subcutaneously.

In some embodiments, the oligonucleotide, oligonucleotide conjugate or pharmaceutical composition of the invention is administered at a dose of 0.1-15 mg/kg, such as from 0.2-10 mg/kg, such as from 0.25-5 mg/kg. The administration can be once a week, every 2^{nd} week, every third week or even once a month.

Combination Therapies

In some embodiments the oligonucleotide, oligonucleotide conjugate or pharmaceutical composition of the invention is for use in a combination treatment with another therapeutic agent. The therapeutic agent can for example be the standard of care for the diseases or disorders described above.

### List of Embodiments

1. An antisense oligonucleotide, 10-30 nucleotides in length, wherein said antisense oligonucleotide comprises a contiguous nucleotide sequence 10-30 nucleotides in length, wherein the contiguous nucleotide sequence is at least 90% complementary, such as fully complementary to SEQ ID NO 1, wherein the antisense oligonucleotide is capable of inhibiting the expression of human CARD9 in a cell which is expressing human CARD9; or a pharmaceutically acceptable salt thereof.
2. The antisense oligonucleotide according to embodiment 1, wherein the contiguous nucleotide sequence is at least 90% complementary to SEQ ID NO 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68 or 69.
3. The antisense oligonucleotide according to embodiment 1, wherein the contiguous nucleotide sequence is fully complementary to SEQ ID NO 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68 or 69.
4. The antisense oligonucleotide according to any one of embodiment 1 to 3, wherein the contiguous nucleotide sequence is fully complementary to a region of SEQ ID NO 1, selected from the group consisting of 1-16; 22-48; 51-72; 74-86; 100-114; 123-165; 229-274; 314-328; 330-342; 344-360; 371-403; 432-471; 477-491; 495-507; 534-548; 576-595; 610-622; 636-664; 674-720; 756-775; 785-798; 800-814; 818-849; 851-865; 868-880; 896-937; 948-978; 990-1009; 1012-1042; 1056-1078; 1097-1130; 1132-1144; 1173-1186; 1195-1209; 1211-1233; 1259-1284; 1299-1311; 1335-1350; 1352-1366; 1384-1401; 1403-1422; 1424-1446; 1448-1473; 1485-1522; 1537-1556; 1580-1596; 1598-1623; 1628-1661; 1670-1686; 1700-1731; 1733-1752; 1764-1794; 1805-1828; 1841-1874; 1876-1910; 1918-1942; 1975-1994; 2009-2036; 2055-2078; 2110-2126; 2128-2152; 2154-2206; 2208-2221; 2230-2287; 2301-2320; 2322-2338; 2340-2371; 2396-2418; 2420-2432; 2435-2483; 2485-2506; 2528-2576; 2578-2633; 2635-2693; 2695-2732; 2734-2783; 2806-2849; 2890-2902; 2904-2924; 2936-2958; 2989-3012; 3014-3054; 3056-3073; 3075-3109; 3111-3169; 3204-3306; 3308-3402; 3441-3478; 3667-3695; 3697-3714; 3746-3773; 3775-3800; 3802-3847; 3858-3883; 3885-3913; 3924-3940; 3955-3969; 3971-3983; 3995-4013; 4019-4098; 4107-4133; 4138-4156; 4162-4178; 4192-4206; 4209-4228; 4244-4269; 4271-4288; 4312-4347; 4375-4415; 4454-4483; 4485-4525; 4588-4604; 4606-4618; 4644-4664; 4666-4684; 4718-4758; 4760-4801; 4810-4831; 4842-4860; 4877-4914; 4916-4936; 4938-4957; 4959-4980; 4991-5005; 5015-5038; 5053-5072; 5074-5087; 5118-5157; 5178-5190; 5205-5218; 5260-5275; 5278-5312; 5314-5326; 5345-5383; 5392-5436; 5485-5497; 5531-5546; 5563-5590; 5600-5632; 5634-5668; 5742-5764; 5791-5807; 5819-5839; 5866-5880; 5890-5915; 5917-5942; 5953-5979; 5981-6041; 6043-6061; 6063-6078; 6090-6102; 6144-6159; 6181-6199; 6227-6241; 6252-6279; 6286-6307; 6316-6389; 6391-6438; 6440-6456; 6458-6484; 6486-6532; 6540-6559; 6586-6611; 6627-6642; 6693-6729; 6765-6799; 6843-6874; 6932-6974; 6980-6995; 7015-7036; 7049-7071; 7094-7129; 7131-7144; 7151-7171; 7173-7207; 7209-7233; 7263-7276; 7323-7345; 7353-7410; 7413-7442; 7490-7502; 7508-7531; 7566-7578; 7580-7592; 7627-7654; 7656-7669; 7671-7688; 7705-7718; 7727-7772; 7774-7787; 7795-7823; 7838-7869; 7873-7903; 7915-7930; 7936-7958; 7960-7984; 7986-7998; 8005-8026; 8028-8045; 8066-8079; 8082-8136; 8138-8151; 8170-8183; 8211-8230; 8232-8263; 8265-8279; 8322-8362; 8381-8404; 8439-8465; 8492-8524; 8535-8552; 8635-8648; 8733-8745; 8768-8784; 8794-8807; 8811-8838; 8843-8872; 8910-8952; 8959-8976; 8983-9010; 9027-9042; 9044-9057; 9078-9102; 9111-9151; 9153-9175; 9186-9243; 9256-9272; 9278-9293; 9295-9310; 9312-9327; 9348-9361; 9363-9400; 9402-9429; 9438-9483; 9498-9521; 9549-9567; 9574-9592; 9594-9623; 9640-9668; and 9701-9726.
5. The antisense oligonucleotide according to any one of embodiment 1-4, wherein the antisense oligonucleotide is a gapmer oligonucleotide comprising a contiguous nucleotide sequence of formula 5′-F-G-F′-3′, where region F and F′ independently comprise 1-8 sugar modified nucleosides, and G is a region between 5 and 16 nucleosides which are capable of recruiting RNaseH.
6. The antisense oligonucleotide according to embodiment 5, wherein the sugar modified nucleosides of region F and F′ are independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.
7. The antisense oligonucleotide according to embodiment 5 or 6, wherein region G comprises 5-16 contiguous DNA nucleosides.
8. The antisense oligonucleotide according to any one of embodiment 1-7, wherein the antisense oligonucleotide is a LNA gapmer oligonucleotide.
9. The antisense oligonucleotide according to any one of embodiment 5-8, wherein the LNA nucleosides are beta-D-oxy LNA nucleosides.
10. The antisense oligonucleotide according to any one of embodiment 1-9, wherein the internucleoside linkages between the contiguous nucleotide sequence are phosphorothioate internucleoside linkages.
11. The antisense oligonucleotide according to any one of embodiment 1-10, wherein the oligonucleotide comprises a contiguous nucleotide sequence selected from the group consisting of SEQ ID NO 70 to SEQ ID NO: 577.
12. The antisense oligonucleotide according to any one of embodiment 1-11, wherein the oligonucleotide is an oligonucleotide compound selected from the oligonucleotide compounds shown in Table 2, wherein a capital letter represents a LNA nucleoside, a lower case letter represents a DNA nucleoside.
13. The antisense oligonucleotide according to any one of embodiment 1-12, wherein the oligonucleotide is an oligonucleotide compound selected from the oligonucleotide compounds shown in Table 2, wherein a capital letter represents a beta-D-oxy LNA nucleoside, a lower case letter represents a DNA nucleoside, wherein each LNA cytosine is 5-methyl cytosine, and wherein the internucleoside linkages between the nucleosides are phosphorothioate internucleoside linkages.
14. A conjugate comprising the oligonucleotide according to any one of embodiment 1-13, and at least one conjugate moiety covalently attached to said oligonucleotide.
15. A pharmaceutical composition comprising the oligonucleotide of embodiment 1-14 or the conjugate of embodiment 14 and a pharmaceutically acceptable diluent, solvent, carrier, salt and/or adjuvant.
16. An in vivo or in vitro method for modulating CARD9 expression in a target cell which is expressing CARD9, said method comprising administering an oligonucleotide of any one of embodiment 1-13, the conjugate according to embodiment 14, or the pharmaceutical composition of embodiment 15 in an effective amount to said cell.
17. A method for treating or preventing a disease comprising administering a therapeutically or prophylactically effective amount of an oligonucleotide of any one of embodiment 1-13 or the conjugate according to embodiment 14 or the pharmaceutical composition of embodiment 15 to a subject suffering from or susceptible to the disease.
18. The method of embodiment 17, wherein the disease is selected from the group consisting of inflammatory bowel disease, pancreatitis, IgA nephropathy, primary sclerosing cholangitis, cardiovascular disease, cancer and diabetes.
19. The oligonucleotide of any one of embodiment 1-13 or the conjugate according to embodiment 14 or the pharmaceutical composition of embodiment 15 for use in medicine.
20. The oligonucleotide of any one of embodiment 1-13 or the conjugate according to embodiment 15 or the pharmaceutical composition of embodiment 15 for use in the treatment or prevention of a disease selected from the group consisting of inflammatory bowel disease, pancreatitis, IgA nephropathy, primary sclerosing cholangitis, cardiovascular disease, cancer and diabetes.
21. Use of the oligonucleotide of embodiment 1-13 or the conjugate according to embodiment 14 or the pharmaceutical composition of embodiment 15, for the preparation of a medicament for treatment or prevention of a disease selected from the group consisting of inflammatory bowel disease, pancreatitis, IgA nephropathy, primary sclerosing cholangitis, cardiovascular disease, cancer and diabetes.

### List of Items

1. An LNA antisense oligonucleotide, 12-24 nucleosides in length, wherein said LNA antisense oligonucleotide comprises a contiguous nucleotide sequence comprising at least 10 contiguous nucleotides present in any one of SEQ ID NO 70 to SEQ ID NO: 577, wherein the antisense oligonucleotide is capable of inhibiting the expression of human CARD9 in a cell which is expressing human CARD9; or a pharmaceutically acceptable salt thereof.
2. The LNA antisense oligonucleotide according to item 1, wherein said LNA antisense oligonucleotide comprises a contiguous nucleotide sequence comprising at least 12 contiguous nucleotides present in any one of SEQ ID NO 70 to SEQ ID NO: 577.
3. The LNA antisense oligonucleotide according to item 1, wherein said LNA antisense oligonucleotide comprises a contiguous nucleotide sequence comprising at least 14 contiguous nucleotides present in any one of SEQ ID NO 70 to SEQ ID NO: 577.
4. The LNA antisense oligonucleotide according to any one of items 1-3, wherein the antisense oligonucleotide is a gapmer oligonucleotide comprising a contiguous nucleotide sequence of formula 5′-F-G-F′-3′, where region F and F′ independently comprise 1-8 sugar modified nucleosides, and G is a region between 5 and 16 nucleosides which are capable of recruiting RNaseH.
5. The LNA antisense oligonucleotide according to item 4, wherein the sugar modified nucleosides of region F and F′ are independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.
6. The LNA antisense oligonucleotide according to item 4 or 5, wherein region G comprises 5-16 contiguous DNA nucleosides.
7. The LNA antisense oligonucleotide according to any one of items 1-6, wherein the antisense oligonucleotide is a LNA gapmer oligonucleotide.
8. The LNA antisense oligonucleotide according to any one of items 4-7, wherein the LNA nucleosides are beta-D-oxy LNA nucleosides.
9. The LNA antisense oligonucleotide according to any one of items 1-8, wherein the internucleoside linkages between the contiguous nucleotide sequence are phosphorothioate internucleoside linkages.
10. The LNA antisense oligonucleotide according to any one of items 1-9, wherein the oligonucleotide comprises a contiguous nucleotide sequence selected from the group consisting of SEQ ID NO 70 to SEQ ID NO: 577.
11. The LNA antisense oligonucleotide according to any one of items 1-10, wherein the LNA antisense oligonucleotide is an oligonucleotide compound selected from the oligonucleotide compounds shown in Table 2, wherein a capital letter represents a LNA nucleoside, a lower case letter represents a DNA nucleoside.
12. The LNA antisense oligonucleotide according to any one of items 1-11, wherein the LNA antisense oligonucleotide is an oligonucleotide compound selected from the oligonucleotide compounds shown in Table 2, wherein a capital letter represents a beta-D-oxy LNA nucleoside, a lower case letter represents a DNA nucleoside, wherein each LNA cytosine is 5-methyl cytosine, and wherein the internucleoside linkages between the nucleosides are phosphorothioate internucleoside linkages.
13. A conjugate comprising the oligonucleotide according to any one of items 1-12, and at least one conjugate moiety covalently attached to said oligonucleotide.
14. A pharmaceutical composition comprising the oligonucleotide of item 1-12 or the conjugate of item 13 and a pharmaceutically acceptable diluent, solvent, carrier, salt and/or adjuvant.
15. An in vivo or in vitro method for modulating CARD9 expression in a target cell which is expressing CARD9, said method comprising administering an oligonucleotide of any one of items 1-12, the conjugate according to item 13, or the pharmaceutical composition of item 14 in an effective amount to said cell.
16. A method for treating or preventing a disease comprising adm, inistering a therapeutically or prophylactically effective amount of an oligonucleotide of any one of items 1-12 or the conjugate according to item 13 or the pharmaceutical composition of item 14 to a subject suffering from or susceptible to the disease.
17. The method of item 16, wherein the disease is selected from the group consisting of inflammatory bowel disease, pancreatitis, IgA nephropathy, primary sclerosing cholangitis, cardiovascular disease, cancer and diabetes.
18. The oligonucleotide of any one of items 1-12 or the conjugate according to item 13 or the pharmaceutical composition of item 14 for use in medicine.
19. The oligonucleotide of any one of items 1-12 or the conjugate according to item 13 or the pharmaceutical composition of item 14 for use in the treatment or prevention of a disease selected from the group consisting of inflammatory bowel disease, pancreatitis, IgA nephropathy, primary sclerosing cholangitis, cardiovascular disease, cancer and diabetes.
20. Use of the oligonucleotide of item 1-12 or the conjugate according to item 13 or the pharmaceutical composition of item 14, for the preparation of a medicament for treatment or prevention of a disease selected from the group consisting of inflammatory bowel disease, pancreatitis, IgA nephropathy, primary sclerosing cholangitis, cardiovascular disease, cancer and diabetes.
21. The method of item 17, the oligonucleotide of item 19, or the use of item 20, wherein the disease is inflammatory bowel disease.

### Example 1 Testing In Vitro Efficacy of LNA Oligonucleotides in the THP-1 Cell Line at 5 μM and 25 μM

An oligonucleotide screen was performed in the human cell line using the LNA oligonucleotides in table 2 (see compounds listed in column “Oligonucleotide compounds”) targeting different regions of SEQ ID NO: 1 (see Table 1). The human cell line THP-1 was purchased from ECACC (catalog no.: 88081201, see Table 4), maintained as recommended by the supplier in a humidified incubator at 37° C. with 5% C02. For the screening assays, cells were seeded in round bottom 96 multi well plates in media recommended by the supplier (see Table 4). The number of cells/well was optimized to 50.000 cells per well.

Cells were seeded and oligonucleotide added in concentration of 5 or 25 μM (dissolved in PBS). Three days after addition of the oligonucleotide, the cells were harvested.

RNA was extracted using the Qiagen RNeasy 96 kit (74182), according to the manufacturer's instructions including DNase treatment step. cDNA synthesis and qPCR was performed using qScript XLT one-step RT-qPCR ToughMix Low ROX, 95134-100 (Quanta Biosciences). Target transcript levels were quantified using a FAM labeled qPCR assay from Integrated DNA Technologies in a multiplex reaction with a VIC labelled GAPDH control from Thermo Fischer Scientific. qPCR primer assays for the target transcript of interest CARD9 (Hs.Pt.58.19155478, FAM), and a house keeping gene GAPDH (4326137E VIC-MGB probe). A technical duplex set up was used, n=1 biological replicate.

The relative CARD9 mRNA expression levels are shown in Table 3 as % of control (PBS-treated cells) i.e. the lower the value the larger the inhibition. “Gene exp.5” and “Gene exp.25” are CARD9 mRNA expressions level after treatment with 5 μM or 25 μM compound.

TABLE 3
Results for tested oligonucleotide compounds
(for more information on the compounds, see Table 2):

| | Gene | Gene |
|---|---|---|
| CMP_ID_NO | exp.5 | exp.25 |
| 70_1 | 72 | 61.4 |
| 71_1 | 69.8 | 66.2 |
| 72_1 | 76.1 | 68.3 |
| 73_1 | 68.9 | 60.8 |
| 74_1 | 66.6 | 62.7 |
| 75_1 | 85.2 | 71.6 |
| 76_1 | 80.7 | 81.7 |
| 77_1 | 79.1 | 61.6 |
| 77_2 | 87.6 | 63.9 |
| 78_1 | 81.6 | 78.8 |
| 79_1 | 85.9 | 65.1 |
| 80_1 | 83.6 | 60.1 |
| 81_1 | 88.5 | 66.5 |
| 82_1 | 85.3 | 69.7 |
| 83_1 | 89.8 | 78 |
| 84_1 | 89.9 | 92.1 |
| 85_1 | 97.4 | 101.2 |
| 86_1 | 108.3 | 91.4 |
| 87_1 | 89 | 63.9 |
| 88_1 | 86.8 | 70.6 |
| 89_1 | 97.7 | 81 |
| 90_1 | 87.1 | 73.3 |
| 90_2 | 88.4 | 69.1 |
| 91_1 | 89.7 | 81.4 |
| 91_2 | 88.8 | 72.7 |
| 92_1 | 80 | 77 |
| 93_1 | 89.1 | 67.2 |
| 94_1 | 84.3 | 65.8 |
| 95_1 | 86.2 | 73.1 |
| 95_2 | 90.8 | 70.5 |
| 96_1 | 82.5 | 75.3 |
| 96_2 | 87.2 | 63.6 |
| 97_1 | 81.6 | 68.7 |
| 98_1 | 80.7 | 69.8 |
| 99_1 | 83.5 | 72.6 |
| 99_2 | 84.3 | 66.2 |
| 100_1 | 82.9 | 62.7 |
| 100_2 | 91.2 | 83.8 |
| 100_3 | 82.4 | 77.9 |
| 101_1 | 86.6 | 84.1 |
| 102_1 | 84.2 | 67.2 |
| 103_1 | 87.7 | 84 |
| 104_1 | 78.7 | 62.5 |
| 104_2 | 85.1 | 63.1 |
| 105_1 | 78.4 | 72.9 |
| 106_1 | 79.6 | 60.1 |
| 107_1 | 79.8 | 67.8 |
| 107_2 | 78.4 | 69 |
| 108_1 | 84 | 68.2 |
| 108_2 | 84.4 | 87.1 |
| 109_1 | 81.8 | 61.2 |
| 110_1 | 90.9 | 63.6 |
| 111_1 | 85.1 | 61.4 |
| 112_1 | 80.3 | 62.2 |
| 113_1 | 81.8 | 70.5 |
| 114_1 | 86.1 | 66 |
| 115_1 | 106.5 | 102.4 |
| 115_2 | 74.1 | 61.2 |
| 116_1 | 101.9 | 73.9 |
| 117_1 | 85.9 | 66.7 |
| 118_1 | 69.7 | 73 |
| 119_1 | 72.9 | 68.2 |
| 119_2 | 86.7 | 83.3 |
| 120_1 | 80.1 | 64.5 |
| 120_2 | 92.4 | 77.2 |
| 121_1 | 87.7 | 83.4 |
| 122_1 | 83.5 | 65.4 |
| 122_2 | 75.5 | 74.1 |
| 123_1 | 75 | 68 |
| 124_1 | 72.4 | 78.1 |
| 125_1 | 71.9 | 62.3 |
| 126_1 | 79.3 | 70.5 |
| 126_2 | 84.4 | 68.3 |
| 127_1 | 78.1 | 64.8 |
| 127_2 | 99.8 | 63.4 |
| 128_1 | 78 | 75.6 |
| 129_1 | 77.3 | 62 |
| 129_2 | 81.3 | 67.3 |
| 130_1 | 74.5 | 77.4 |
| 131_1 | 110 | 66.2 |
| 131_2 | 93.7 | 84.5 |
| 132_1 | 90.6 | 93.4 |
| 133_1 | NA | 76.3 |
| 133_2 | 97.3 | 89.8 |
| 134_1 | 86.9 | 73.6 |
| 134_2 | 79.8 | 73.2 |
| 135_1 | NA | 72.9 |
| 136_1 | 92.8 | 82.5 |
| 137_1 | 82.9 | 73 |
| 137_2 | 90.9 | 63.9 |
| 138_1 | 83.2 | 69.6 |
| 139_1 | 102.9 | 90.9 |
| 140_1 | 90.4 | 65.8 |
| 141_1 | 85.9 | 65.2 |
| 142_1 | 85.1 | 65.6 |
| 142_2 | 78.9 | 68.7 |
| 143_1 | 86.6 | 80.5 |
| 144_1 | 83.5 | 65.8 |
| 144_2 | 103.6 | 96.4 |
| 145_1 | 82 | 66 |
| 146_1 | 72.7 | 62.6 |
| 147_1 | 84.4 | NA |
| 148_1 | 88.2 | 73.7 |
| 149_1 | 82.3 | 67.3 |
| 150_1 | 85.5 | 78.1 |
| 151_1 | 82.9 | 83 |
| 152_1 | 73.8 | 65.2 |
| 153_1 | 76.5 | 63.9 |
| 154_1 | 78.6 | 77.4 |
| 155_1 | 77.5 | 61.1 |
| 156_1 | 81.8 | 65.5 |
| 156_2 | 84.6 | 78.7 |
| 157_1 | 76.2 | 66.1 |
| 158_1 | 71.7 | 66.8 |
| 159_1 | 90.1 | 83.2 |
| 160_1 | 88.4 | 70.8 |
| 161_1 | 86.6 | 63.6 |
| 162_1 | 93.5 | 81.6 |
| 163_1 | 96 | 84.1 |
| 164_1 | 93.8 | 85.4 |
| 165_1 | 93.8 | 77.6 |
| 166_1 | 91.5 | 78.7 |
| 167_1 | 97.6 | 82.6 |
| 168_1 | 76 | 65.7 |
| 169_1 | 94.3 | 77.2 |
| 170_1 | 83.8 | 76.7 |
| 170_2 | 84.4 | 64.9 |
| 171_1 | 67.8 | 67.6 |
| 171_2 | 84.7 | 77.1 |
| 172_1 | 72.3 | 60.3 |
| 172_2 | 109.4 | 96.6 |
| 173_1 | 80.2 | 72.6 |
| 174_1 | 94.4 | 81.5 |
| 175_1 | 64.7 | 62.4 |
| 175_2 | 101 | 90.5 |
| 175_3 | 79.7 | 72.6 |
| 176_1 | 80.1 | 85.6 |
| 177_1 | 87.7 | 73.1 |
| 177_2 | 77.9 | 78.3 |
| 178_1 | 83.9 | 73.6 |
| 179_1 | 67.7 | 62.9 |
| 180_1 | 90.2 | 74.9 |
| 180_2 | 85.5 | 85.1 |
| 181_1 | 66.1 | 65.9 |
| 182_1 | 86 | 67.7 |
| 182_2 | 81.6 | 77.5 |
| 182_3 | 92.3 | 86.3 |
| 183_1 | 87 | 63.9 |
| 183_2 | 72.1 | 65.4 |
| 184_1 | 79.4 | 65.6 |
| 185_1 | 77.7 | 62.5 |
| 186_1 | 78.6 | NA |
| 186_2 | 83.7 | 72 |
| 187_1 | 68.5 | 61.5 |
| 187_2 | 84.8 | 65.9 |
| 188_1 | NA | 95.8 |
| 188_2 | 69.3 | 66.1 |
| 188_3 | 70.4 | 70.4 |
| 189_1 | 72.4 | 65.8 |
| 190_1 | 81.7 | 72.3 |
| 190_2 | 80.1 | 85.1 |
| 191_1 | 83.1 | 85.9 |
| 192_1 | 89.3 | 82 |
| 192_2 | 101.8 | NA |
| 193_1 | NA | 100.2 |
| 193_2 | 73.4 | 71.6 |
| 194_1 | 90.2 | 82.7 |
| 194_2 | 93.9 | 86.6 |
| 195_1 | NA | 87.5 |
| 195_2 | 102.6 | 89.7 |
| 195_3 | 86.9 | 74.6 |
| 196_1 | 101.7 | 91.6 |
| 197_1 | 91 | 101.9 |
| 197_2 | 95 | 88.4 |
| 198_1 | 87.9 | 79.7 |
| 199_1 | 77.7 | 63.4 |
| 200_1 | 85.9 | 85.1 |
| 201_1 | 82.5 | 86.2 |
| 202_1 | 84.8 | 83.8 |
| 203_1 | 84.3 | 78.7 |
| 204_1 | 82.6 | 80.4 |
| 205_1 | 75 | 61.1 |
| 206_1 | 81.4 | 74.1 |
| 207_1 | 85 | 71.1 |
| 207_2 | 77.8 | 67.9 |
| 208_1 | 83.7 | 73.9 |
| 209_1 | 89.6 | 98.2 |
| 210_1 | 87.6 | 69.7 |
| 210_2 | 90 | 81 |
| 211_1 | 87.8 | 82.5 |
| 211_2 | 92.9 | 95.4 |
| 212_1 | 94.4 | 84.6 |
| 213_1 | 97.4 | 74.7 |
| 213_2 | 87.2 | 86.6 |
| 214_1 | 90.6 | 87.2 |
| 214_2 | 86.8 | 88.2 |
| 215_1 | 99.7 | 80.6 |
| 215_2 | 93.8 | 90.1 |
| 216_1 | 110.6 | 92.6 |
| 216_2 | 88.1 | 85.4 |
| 217_1 | 97.4 | 104.4 |
| 218_1 | 90.3 | 87.3 |
| 219_1 | 91.9 | 92.3 |
| 220_1 | 100.7 | 99 |
| 221_1 | 93.6 | 115.7 |
| 222_1 | 86 | 86.7 |
| 223_1 | 85 | 69.6 |
| 224_1 | 88.8 | 78 |
| 225_1 | 83.7 | 69.4 |
| 226_1 | 83.6 | 68.1 |
| 227_1 | 68 | NA |
| 228_1 | 77.7 | 63.2 |
| 229_1 | 75.2 | 66.4 |
| 230_1 | 78.2 | 66.1 |
| 231_1 | 75.7 | 62.4 |
| 232_1 | 74.2 | 71.6 |
| 233_1 | 100.9 | 79.1 |
| 234_1 | 89.8 | 84 |
| 235_1 | 82.9 | 78.1 |
| 236_1 | 77.7 | 68.9 |
| 237_1 | 80.4 | 70.8 |
| 238_1 | 82.4 | 73 |
| 239_1 | 87.4 | 87.9 |
| 240_1 | 87.4 | 91.8 |
| 241_1 | 95.8 | 70.9 |
| 242_1 | 110.2 | 87.6 |
| 242_2 | 99 | 90.4 |
| 242_3 | 98 | 80.9 |
| 243_1 | 96.7 | 100.4 |
| 243_2 | 85.3 | 67.2 |
| 244_1 | 116 | 83.7 |
| 245_1 | 99 | 101.6 |
| 245_2 | 85 | 70.1 |
| 246_1 | 105.8 | 85.3 |
| 246_2 | 82.9 | 63.6 |
| 247_1 | 92.5 | 96.6 |
| 248_1 | 91.2 | 80.2 |
| 249_1 | 95 | 72 |
| 250_1 | 88.1 | 87.2 |
| 251_1 | 97.2 | 77.8 |
| 252_1 | 109.9 | 86.2 |
| 252_2 | 80.7 | 82.2 |
| 253_1 | 84.2 | 67.1 |
| 254_1 | 91.8 | 68.8 |
| 255_1 | 70.8 | 74.2 |
| 255_2 | 85 | 95.8 |
| 255_3 | 89.1 | 71.3 |
| 256_1 | 79.7 | 64.1 |
| 257_1 | 84.8 | 81.4 |
| 258_1 | 89.6 | 77.6 |
| 259_1 | 86.6 | 62.6 |
| 260_1 | 84.8 | 60.9 |
| 261_1 | 80.9 | 63.1 |
| 262_1 | 90.9 | 83.9 |
| 263_1 | 76.2 | 67.3 |
| 263_2 | 97.7 | 89 |
| 264_1 | 90.3 | 74.9 |
| 265_1 | 86.3 | 81.5 |
| 266_1 | 91.7 | 74.8 |
| 266_2 | 83.8 | 81.4 |
| 267_1 | 82.7 | 84.6 |
| 268_1 | 96.1 | 86.3 |
| 269_1 | 71.3 | 60.2 |
| 270_1 | 81.3 | 64.1 |
| 271_1 | 78.1 | 60.8 |
| 272_1 | 82.3 | 72.6 |
| 273_1 | 77.3 | 78.9 |
| 274_1 | 87.2 | 77.7 |
| 275_1 | 80.9 | 67.6 |
| 276_1 | 75.4 | 85 |
| 277_1 | 78.1 | 65.3 |
| 278_1 | 78.6 | 66.3 |
| 279_1 | 72.2 | 81 |
| 280_1 | 92.3 | 86.2 |
| 280_2 | 77.5 | 66.8 |
| 281_1 | 80.2 | 90.9 |
| 282_1 | 77.6 | 71.7 |
| 283_1 | 83.4 | 77.2 |
| 284_1 | 77.3 | 76.4 |
| 285_1 | 79 | 61.5 |
| 285_2 | 79.8 | 75.8 |
| 286_1 | 80.4 | 78.7 |
| 287_1 | 76.6 | 71.5 |
| 287_2 | 84.8 | 77 |
| 288_1 | 95.7 | 85 |
| 288_2 | 91.9 | 91.6 |
| 289_1 | 85.8 | 69.9 |
| 289_2 | 92 | 84.4 |
| 290_1 | 71.9 | 66.2 |
| 290_2 | 80.8 | 87.6 |
| 291_1 | 73.7 | 60.6 |
| 292_1 | NA | 63.4 |
| 292_2 | 74.3 | 87.5 |
| 293_1 | 108.1 | 73.4 |
| 294_1 | 81.4 | 72.3 |
| 295_1 | 95.5 | 66.1 |
| 296_1 | 97.9 | 81.4 |
| 296_2 | 93.2 | 74.2 |
| 297_1 | 86.8 | 75 |
| 298_1 | 81.4 | 61.6 |
| 299_1 | 84.3 | 61.8 |
| 300_1 | 77.1 | 72.8 |
| 301_1 | 85.9 | 67.1 |
| 302_1 | 74.3 | 68.6 |
| 303_1 | 88.9 | 81.5 |
| 304_1 | 78 | 64.3 |
| 304_2 | 84.1 | 72.8 |
| 305_1 | 90 | 79.9 |
| 306_1 | 73.4 | 64.2 |
| 307_1 | 104.2 | 86.8 |
| 308_1 | 89.1 | 67.8 |
| 309_1 | 79.9 | 73 |
| 310_1 | 79.1 | 64 |
| 311_1 | 87.5 | 85.5 |
| 312_1 | 89.3 | 70.9 |
| 313_1 | 77.8 | 61.9 |
| 314_1 | 74.2 | 67 |
| 315_1 | 77.2 | 65.6 |
| 316_1 | 72.7 | 63.4 |
| 317_1 | 92.8 | 91.1 |
| 318_1 | 84.5 | 78.5 |
| 319_1 | 87.6 | 65.4 |
| 320_1 | 95.5 | 71.1 |
| 320_2 | 88 | 65.7 |
| 321_1 | 71.6 | 61.1 |
| 322_1 | 89 | 77.2 |
| 323_1 | 93.1 | 76.3 |
| 323_2 | 92.3 | 67.7 |
| 323_3 | 83.3 | 88.4 |
| 324_1 | 95.7 | 85.6 |
| 324_2 | 84.9 | 64.9 |
| 324_3 | 79 | 78 |
| 325_1 | 80.6 | 71.2 |
| 325_2 | 97.3 | 73.2 |
| 325_3 | 79.5 | 65.5 |
| 326_1 | 102.8 | 83 |
| 326_2 | 98.4 | 72.8 |
| 326_3 | 84.3 | 71.4 |
| 327_1 | 79.1 | 66.6 |
| 327_2 | 103.1 | 85.2 |
| 328_1 | 73.7 | 64.2 |
| 328_2 | 90.4 | 86.5 |
| 329_1 | 79 | 74.7 |
| 329_2 | 86.2 | 77.2 |
| 329_3 | 87.6 | 84.7 |
| 330_1 | 86.9 | 76.3 |
| 330_2 | 86.8 | 79.6 |
| 330_3 | 76 | 65.1 |
| 331_1 | 89.9 | 67.3 |
| 331_2 | 84.4 | 65.6 |
| 332_1 | 80.4 | 70.1 |
| 332_2 | 82.2 | 85.8 |
| 333_1 | 85.4 | 76.4 |
| 334_1 | 83.5 | 67.7 |
| 334_2 | 116 | 89 |
| 334_3 | 96.1 | 96.3 |
| 335_1 | 89.1 | 84.8 |
| 336_1 | 97.2 | 76.2 |
| 337_1 | NA | 63.5 |
| 337_2 | 81.6 | 66.4 |
| 338_1 | 107.2 | 75 |
| 338_2 | 91.3 | 64.8 |
| 338_3 | 86.3 | 92.6 |
| 339_1 | 79.4 | 65.5 |
| 339_2 | 82.2 | 77 |
| 339_3 | 88.3 | 107.7 |
| 340_1 | 105.7 | 87.9 |
| 340_2 | 97.1 | 77.8 |
| 340_3 | 84.3 | 77 |
| 341_1 | 91.9 | 76.6 |
| 341_2 | 102.5 | 77.6 |
| 341_3 | 99.6 | 83.4 |
| 342_1 | 87.1 | 75.5 |
| 342_2 | 78.9 | 60.5 |
| 342_3 | 82.6 | 73 |
| 343_1 | 96.9 | 91.9 |
| 343_2 | 92.2 | 79 |
| 343_3 | 91.1 | 81.7 |
| 344_1 | 113.4 | 92 |
| 344_2 | 90.5 | 75.6 |
| 345_1 | NA | 60.5 |
| 345_2 | 90.3 | 79.2 |
| 346_1 | NA | 86.4 |
| 346_2 | 91.7 | 80.6 |
| 347_1 | 93 | 82.4 |
| 348_1 | 93.5 | 95.1 |
| 349_1 | 93.7 | 83.9 |
| 350_1 | 90.9 | 81 |
| 351_1 | 89.9 | 90.1 |
| 352_1 | 88.4 | 95.9 |
| 353_1 | 85.3 | 81.9 |
| 354_1 | 96.2 | 89.1 |
| 355_1 | 97.5 | 84.6 |
| 355_2 | 92.6 | 94.4 |
| 356_1 | 87.5 | 70.9 |
| 357_1 | 81.6 | 61.6 |
| 358_1 | 98.5 | 79.6 |
| 358_2 | 86.9 | 78.7 |
| 359_1 | 87.8 | 82.4 |
| 360_1 | 91.1 | 76.9 |
| 361_1 | 93.1 | 90.8 |
| 362_1 | 93.8 | 90.5 |
| 363_1 | 82.7 | 66.7 |
| 364_1 | 80.8 | 67.3 |
| 365_1 | 83.1 | 75.7 |
| 366_1 | 76.6 | 63.1 |
| 367_1 | 76.4 | 68.6 |
| 368_1 | 77.8 | 64.8 |
| 369_1 | 83.9 | 79.1 |
| 370_1 | 84.9 | 85.3 |
| 371_1 | 86 | 60.4 |
| 372_1 | 70.2 | 61.6 |
| 373_1 | 82.7 | 65.7 |
| 374_1 | 77.5 | 63.9 |
| 375_1 | 76.1 | 61.1 |
| 375_2 | 85.2 | 72.3 |
| 376_1 | 80.5 | 76 |
| 376_2 | 89.8 | 62.6 |
| 377_1 | 86.3 | 81.8 |
| 377_2 | 88.6 | 78.7 |
| 378_1 | 76.9 | 66.9 |
| 379_1 | 82.3 | 66.4 |
| 379_2 | 89.3 | 74.3 |
| 380_1 | 88.3 | 71.3 |
| 381_1 | 79.1 | 61.3 |
| 382_1 | 94.4 | 73.1 |
| 383_1 | 85.8 | 64.8 |
| 384_1 | 90.4 | 64.5 |
| 385_1 | 93 | 93.1 |
| 386_1 | 94.7 | 106.4 |
| 386_2 | 90.6 | 91.6 |
| 387_1 | 79.5 | 69.2 |
| 388_1 | 90.2 | 82.7 |
| 388_2 | 78.7 | 61.4 |
| 389_1 | 87.7 | 65.2 |
| 390_1 | 87 | 61.9 |
| 391_1 | 86.5 | 72.2 |
| 392_1 | 82.7 | 83.4 |
| 393_1 | 90.7 | 63.9 |
| 394_1 | 82.8 | 63.4 |
| 394_2 | 79.9 | 63.9 |
| 395_1 | 85.2 | 64.9 |
| 396_1 | 82.1 | 74.2 |
| 397_1 | 91.5 | 66.7 |
| 398_1 | 88.8 | 70.9 |
| 398_2 | 81.6 | 70.3 |
| 399_1 | 81.9 | 67.3 |
| 400_1 | 84.1 | 67.4 |
| 401_1 | 79.2 | 90.5 |
| 402_1 | 87.6 | 68.6 |
| 403_1 | NA | 81.8 |
| 404_1 | 92.5 | 71.2 |
| 404_2 | 92.2 | 79.8 |
| 405_1 | 91.9 | 69.8 |
| 406_1 | 100.5 | 97.4 |
| 407_1 | 105.1 | NA |
| 408_1 | 79.9 | 80.4 |
| 409_1 | 98.9 | 86.3 |
| 409_2 | 94.8 | 105.1 |
| 409_3 | 88.4 | 78.8 |
| 410_1 | 110 | 98.2 |
| 410_2 | 91 | 94.6 |
| 410_3 | 95.2 | 70.8 |
| 411_1 | 84 | 86.5 |
| 411_2 | 113.5 | 85.4 |
| 411_3 | 85.9 | 90 |
| 412_1 | 88.2 | 69.9 |
| 413_1 | 89.9 | 77.7 |
| 413_2 | 95.3 | 81.3 |
| 414_1 | 97 | 118.7 |
| 414_2 | 87.2 | 74.7 |
| 414_3 | 86.4 | 91 |
| 415_1 | 97.9 | 84.9 |
| 415_2 | 91.1 | 62.7 |
| 416_1 | 94.9 | 112.5 |
| 416_2 | 87.4 | 80.8 |
| 416_3 | 99.2 | 71.7 |
| 417_1 | 79.6 | 91.6 |
| 417_2 | 92.2 | 83.5 |
| 417_3 | 81.1 | 77.8 |
| 418_1 | 81.9 | 85.7 |
| 419_1 | 94.4 | 82.5 |
| 420_1 | 72.9 | 73.2 |
| 421_1 | 79.2 | 64 |
| 422_1 | 80.1 | 66.7 |
| 423_1 | 99.9 | 94.8 |
| 423_2 | 87.9 | 81 |
| 424_1 | 87.6 | 88.2 |
| 425_1 | 87.6 | 85 |
| 426_1 | 85.4 | 89.2 |
| 427_1 | 88.1 | 85 |
| 428_1 | 85.9 | 73.9 |
| 429_1 | 79.4 | 74.5 |
| 429_2 | 84 | 77.6 |
| 430_1 | 88.5 | 66 |
| 431_1 | 94.6 | 85.3 |
| 432_1 | 85.4 | 75.2 |
| 433_1 | 70.4 | 73.5 |
| 433_2 | 92.7 | 101.3 |
| 434_1 | 73.8 | 72.8 |
| 434_2 | 84.6 | 83.1 |
| 435_1 | 79.3 | 62.9 |
| 435_2 | 86.9 | 74 |
| 436_1 | 90.6 | 60 |
| 437_1 | 90.3 | 65.6 |
| 438_1 | 68 | 63.8 |
| 438_2 | 86.4 | 71.9 |
| 439_1 | 68.8 | 62.2 |
| 440_1 | 79.3 | 71.9 |
| 441_1 | 75.1 | 70.9 |
| 442_1 | 93.5 | 90.1 |
| 443_1 | 86.9 | 70 |
| 444_1 | 80 | 79.7 |
| 445_1 | 82 | 69.7 |
| 446_1 | 87.1 | 64.7 |
| 447_1 | 78.6 | 60.1 |
| 447_2 | 84.1 | 81.7 |
| 447_3 | 94.4 | 78.4 |
| 448_1 | 90.4 | 88.5 |
| 449_1 | 83.5 | 72.1 |
| 449_2 | 92.6 | 77.6 |
| 450_1 | 86.2 | 77.7 |
| 451_1 | 84.8 | 67.9 |
| 451_2 | 100.2 | 86.5 |
| 452_1 | 88.9 | 71.1 |
| 452_2 | 99.3 | 83.8 |
| 452_3 | 106.3 | 97.8 |
| 453_1 | 81.5 | 62.8 |
| 454_1 | 90.6 | 83.4 |
| 454_2 | 82.7 | 66 |
| 455_1 | 91.6 | 68.7 |
| 456_1 | 86.5 | 62.9 |
| 457_1 | 90.7 | 76.5 |
| 458_1 | 72.1 | 69.5 |
| 459_1 | 81.1 | 64.6 |
| 460_1 | 99.5 | 83.2 |
| 461_1 | 80 | 67.2 |
| 462_1 | 89.8 | 72 |
| 462_2 | 91.9 | 78.1 |
| 463_1 | 83.3 | 67.3 |
| 464_1 | 75.3 | 65.4 |
| 465_1 | 101.1 | 63.1 |
| 466_1 | 100.3 | 73.7 |
| 467_1 | 73.6 | 63.1 |
| 468_1 | 86.1 | 65.4 |
| 469_1 | 85.3 | 76.9 |
| 469_2 | 82.7 | 75.6 |
| 469_3 | 91.2 | 77.2 |
| 470_1 | 81.4 | 61.7 |
| 470_2 | 73.7 | 67.7 |
| 470_3 | 84.5 | 63.1 |
| 471_1 | 86 | 65.3 |
| 472_1 | 92.1 | 96.6 |
| 473_1 | 79.1 | 64.1 |
| 474_1 | 78.5 | 61.4 |
| 474_2 | 79.9 | 60.7 |
| 475_1 | 80 | 65.7 |
| 476_1 | 76.8 | 73.4 |
| 476_2 | 93.2 | 86.6 |
| 477_1 | 83.2 | 75.2 |
| 478_1 | 82.2 | 65.4 |
| 479_1 | 85.3 | 61.7 |
| 479_2 | 87.7 | 69.6 |
| 480_1 | 98.7 | 98.7 |
| 480_2 | 74.7 | 68.6 |
| 480_3 | 87.8 | 71.8 |
| 481_1 | 80.7 | 67.9 |
| 482_1 | 99.1 | 114.9 |
| 482_2 | 79.6 | 67.8 |
| 482_3 | 98 | 79.7 |
| 483_1 | 85.3 | 75.4 |
| 484_1 | 89.1 | 91.2 |
| 484_2 | 81.5 | 63 |
| 484_3 | 83.6 | 62 |
| 485_1 | 89.2 | 81.3 |
| 485_2 | 85.5 | 67.1 |
| 486_1 | 94 | 79 |
| 487_1 | 83.2 | 68.1 |
| 488_1 | 91.4 | 75.7 |
| 489_1 | 81.6 | 76.3 |
| 489_2 | 85.4 | 79 |
| 489_3 | 89.5 | 83.6 |
| 490_1 | 93.9 | 95.7 |
| 490_2 | 83.4 | 73.3 |
| 491_1 | 80.2 | 67.2 |
| 492_1 | 97.7 | 112.1 |
| 492_2 | 82.9 | 74.3 |
| 493_1 | 85 | 76.8 |
| 494_1 | 90.5 | 82.8 |
| 494_2 | 78.4 | 61.1 |
| 495_1 | 79.5 | 78.3 |
| 495_2 | 89.5 | 71.3 |
| 496_1 | 95.7 | 97.3 |
| 496_2 | 83 | 67.6 |
| 497_1 | 84.6 | 72.5 |
| 497_2 | 72.2 | 60.6 |
| 498_1 | 74.8 | 77.1 |
| 498_2 | 76.1 | 70.5 |
| 499_1 | 81.8 | 74.1 |
| 499_2 | 75.1 | 60.1 |
| 499_3 | 93.8 | 93.9 |
| 500_1 | 75.2 | 78.2 |
| 500_2 | 85.4 | 78.9 |
| 500_3 | 86.1 | 72.2 |
| 501_1 | 97.5 | 106.5 |
| 501_2 | 89.4 | 77.2 |
| 501_3 | 93.4 | 85.6 |
| 502_1 | 85 | 76 |
| 502_2 | 81.1 | 63.6 |
| 503_1 | 100.8 | 89 |
| 503_2 | 87.9 | 74.7 |
| 504_1 | 86.3 | 70.7 |
| 504_2 | 72.7 | 61 |
| 505_1 | 102.9 | 94.3 |
| 506_1 | 71.1 | 66.6 |
| 507_1 | 81.5 | 68.8 |
| 507_2 | 76.2 | 60.9 |
| 508_1 | 81.2 | 66.1 |
| 509_1 | 104.3 | 112.4 |
| 509_2 | 93.7 | 69.7 |
| 509_3 | 98 | 75.5 |
| 510_1 | 77.4 | 64.7 |
| 511_1 | 83.8 | 80.8 |
| 511_2 | 88 | 73.4 |
| 511_3 | 104 | 85.1 |
| 512_1 | 91.2 | 69.8 |
| 513_1 | 73.3 | 70.8 |
| 513_2 | 97.2 | 112.7 |
| 514_1 | 73.1 | 61.6 |
| 515_1 | 99.1 | 89.7 |
| 515_2 | 84.5 | 63.9 |
| 516_1 | 99 | 80.9 |
| 517_1 | 93 | 80.1 |
| 517_2 | 83.8 | 78.9 |
| 518_1 | 86.3 | 74.2 |
| 519_1 | 88.3 | 71.6 |
| 519_2 | 85 | 62.2 |
| 519_3 | 67.7 | 60.5 |
| 520_1 | 91.7 | 76.6 |
| 521_1 | 80.6 | 63.7 |
| 522_1 | 88.9 | 74.1 |
| 523_1 | 93.3 | 68.8 |
| 524_1 | 80.6 | 63.7 |
| 524_2 | 96.2 | 87.3 |
| 525_1 | 96.3 | 81.6 |
| 525_2 | 91.3 | 65.7 |
| 526_1 | 86.9 | 73.3 |
| 526_2 | 86.3 | 75.2 |
| 527_1 | 87 | 72.6 |
| 528_1 | 83.3 | 83.2 |
| 528_2 | 91.7 | 80.5 |
| 528_3 | 92.4 | 81.5 |
| 529_1 | 90.2 | 80.1 |
| 530_1 | 96.4 | 95.4 |
| 531_1 | 109.7 | 114.6 |
| 531_2 | 95.3 | 113 |
| 532_1 | 99.1 | 97.4 |
| 533_1 | 80.8 | 74.4 |
| 534_1 | 95.3 | 84.7 |
| 535_1 | 80.5 | 80 |
| 536_1 | 101.3 | 82.3 |
| 536_2 | 91.5 | 88.3 |
| 537_1 | 82.2 | 72.8 |
| 538_1 | 95.5 | 81 |
| 538_2 | 86.1 | 66.7 |
| 538_3 | 89.5 | 87.9 |
| 539_1 | 89.6 | 70.9 |
| 540_1 | 91.9 | 95.4 |
| 541_1 | 83.1 | 83.1 |
| 542_1 | 103.3 | 100.6 |
| 542_2 | 88.1 | 67.9 |
| 542_3 | 112.1 | 90.7 |
| 543_1 | 91 | 80.2 |
| 543_2 | 88.3 | 79.9 |
| 544_1 | 92.9 | 89.7 |
| 545_1 | 86.4 | 96.9 |
| 546_1 | 89.4 | 72.9 |
| 547_1 | 81.1 | 83.7 |
| 548_1 | 84.2 | 75.4 |
| 549_1 | 84.5 | 85.2 |
| 550_1 | 88.5 | 84.6 |
| 551_1 | 88.4 | 104.9 |
| 552_1 | 96.5 | 78.3 |
| 552_2 | 83.5 | 69.9 |
| 552_3 | 81.8 | 80.8 |
| 553_1 | 86.7 | 83.2 |
| 553_2 | 88.2 | 97 |
| 554_1 | 89.8 | 86.8 |
| 555_1 | 93.8 | 108.4 |
| 556_1 | 86.3 | 79.6 |
| 556_2 | 87.4 | 71.3 |
| 557_1 | 88.7 | 60.8 |
| 558_1 | 96.3 | 115.2 |
| 558_2 | 83.5 | 63.5 |
| 559_1 | 102.7 | 98.7 |
| 560_1 | 91.3 | 78.6 |
| 561_1 | 91.5 | 73.6 |
| 561_2 | 86.4 | 69.7 |
| 562_1 | 93.9 | 83.4 |
| 563_1 | 78 | 62.3 |
| 564_1 | 81.8 | 66.7 |
| 565_1 | 74.7 | 62.4 |
| 565_2 | 85.5 | 69.3 |
| 566_1 | 106.9 | 106.8 |
| 567_1 | 85.8 | 88.7 |
| 568_1 | 75.9 | 62.7 |
| 568_2 | 77.1 | 70.4 |
| 569_1 | 83.4 | 82.8 |
| 570_1 | 81.2 | 64.2 |
| 571_1 | 72.5 | 65.9 |
| 572_1 | 90.4 | 89.4 |
| 573_1 | 78.1 | 66.3 |
| 574_1 | 91.9 | 75.8 |
| 575_1 | 84.4 | 64.7 |
| 575_2 | 64.3 | 65.8 |
| 576_1 | 80.8 | 81.2 |
| 576_2 | 70.8 | 62.2 |
| 577_1 | 85.3 | 75.8 |

### Cell Lines

TABLE 4
Details in relation to the cell lines used in Example 1

| | | | | | | Hours of | |
|---|---|---|---|---|---|---|---|
| | | | | | | cell | |
| | | | | Cells/well | | incubation | |
| Cell lines | | | | (96 well | | prior to | Days of |
| Name | Vendor | Cat. no. | Cell medium* | plate) | Plates | treatment | treatment |
| THP-1 | ECACC | 88081201 | RPMI 1640 | 50.000 | Nunc | 0 | 0 |
| | | | (cat. no. R2405), | | (Cat. no. | | |
| | | | 10% FBS (cat. no. | | 168136) | | |
| | | | F7524), 25 μg/ml | | | | |
| | | | Gentamicin | | | | |
| | | | cat. no. G1397) | | | | |

*All medium and additives were purchased from Sigma Aldrich

1. An antisense oligonucleotide, 12-24 nucleosides in length, wherein said antisense oligonucleotide comprises a contiguous nucleotide sequence comprising at least 10 contiguous nucleotides present in any one of SEQ ID NO 70 to SEQ ID NO: 577, wherein the antisense oligonucleotide is capable of inhibiting the expression of human CARD9 in a cell which is expressing human CARD9; or a pharmaceutically acceptable salt thereof.

2. The antisense oligonucleotide according to claim 1, wherein said antisense oligonucleotide comprises a contiguous nucleotide sequence comprising at least 12 contiguous nucleotides present in any one of SEQ ID NO 70 to SEQ ID NO: 577.

3. The antisense oligonucleotide according to claim 1, wherein said antisense oligonucleotide comprises a contiguous nucleotide sequence comprising at least 14 contiguous nucleotides present in any one of SEQ ID NO 70 to SEQ ID NO: 577.

4. The antisense oligonucleotide according to claim 1, wherein the antisense oligonucleotide is a gapmer oligonucleotide comprising a contiguous nucleotide sequence of formula 5′-F-G-F′-3′, where region F and F′ independently comprise 1-8 sugar modified nucleosides, and G is a region between 5 and 16 nucleosides which are capable of recruiting RNaseH.

5. The antisense oligonucleotide according to claim 4, wherein the sugar modified nucleosides of region F and F′ are independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.

6. The antisense oligonucleotide according to claim 1, wherein region G comprises 5-16 contiguous DNA nucleosides.

7. The antisense oligonucleotide according to claim 1, wherein the antisense oligonucleotide is a LNA antisense oligonucleotide.

8. The antisense oligonucleotide according to claim 1, wherein the antisense oligonucleotide is a LNA gapmer oligonucleotide.

9. The antisense oligonucleotide according to claim 1, wherein the LNA nucleosides are beta-D-oxy LNA nucleosides.

10. The antisense oligonucleotide according to claim 1, wherein the internucleoside linkages between the contiguous nucleotide sequence are phosphorothioate internucleoside linkages.

11. The antisense oligonucleotide according to claim 1, wherein the oligonucleotide comprises a contiguous nucleotide sequence selected from the group consisting of SEQ ID NO 70 to SEQ ID NO: 577.

12. The antisense oligonucleotide according to claim 1, wherein the antisense oligonucleotide is an oligonucleotide compound selected from the oligonucleotide compounds shown in Table 2, wherein a capital letter represents a nucleoside, and a lower case letter represents a DNA nucleoside.

13. The antisense oligonucleotide according to claim 1, wherein the antisense oligonucleotide is an oligonucleotide compound selected from the oligonucleotide compounds shown in Table 2, wherein a capital letter represents a beta-D-oxy LNA nucleoside, a lower case letter represents a DNA nucleoside, and a superscript m before a lower case c represents a 5-methyl cytosine DNA nucleoside, wherein each LNA cytosine is 5-methyl cytosine, and wherein the internucleoside linkages between the nucleosides are phosphorothioate internucleoside linkages.

14. A conjugate comprising the oligonucleotide according to claim 1, and at least one conjugate moiety covalently attached to said oligonucleotide.

15. A pharmaceutical composition comprising the oligonucleotide of claim 1 and a pharmaceutically acceptable diluent, solvent, carrier, salt and/or adjuvant.

16. An in vivo or in vitro method for modulating CARD9 expression in a target cell which is expressing CARD9, said method comprising administering an oligonucleotide according to claim 1 in an effective amount to said cell.

17. A method for treating or preventing a disease comprising administering a therapeutically or prophylactically effective amount of an oligonucleotide according to claim 1 to a subject suffering from or susceptible to the disease.

18. The method of claim 17, wherein the disease is selected from the group consisting of inflammatory bowel disease, pancreatitis, IgA nephropathy, primary sclerosing cholangitis, cardiovascular disease, cancer and diabetes.

19. The oligonucleotide according to claim 1 for use in medicine.

20. The oligonucleotide according to claim 1 for use in the treatment or prevention of a disease selected from the group consisting of inflammatory bowel disease, pancreatitis, IgA nephropathy, primary sclerosing cholangitis, cardiovascular disease, cancer and diabetes.

21. Use of the oligonucleotide according to claim 1, for the preparation of a medicament for treatment or prevention of a disease selected from the group consisting of inflammatory bowel disease, pancreatitis, IgA nephropathy, primary sclerosing cholangitis, cardiovascular disease, cancer and diabetes.

22. The oligonucleotide of claim 20, wherein the disease is inflammatory bowel disease.
